(19) United States
(12) Patent Application Publication
(10) Pub. No.: US 2003/0124599 A1
(43) Pub. Date: Jul. 3, 2003

(54) Biochemical analysis system with combinatorial chemistry applications

(76) Inventors: Shiping Chen, Fremont, CA (US); Yuling Luo, Castro Valley, CA (US); Anthony C. Chen, Sunnyvale, CA (US); Theo Nikiforov, San Jose, CA (US); Jianming Xiao, Fremont, CA (US); Quan Nguyen, Pleasant Hills, CA (US)

Correspondence Address: Charles D. Holland, Morrison & Foerster LLP, 755 Page Mill Road, Palo Alto, CA, 94304-1018, US

(21) Appl. No.: 10/295,230

(22) Filed: Nov. 14, 2002

Related U.S. Application Data
(60) Provisional application No. 60/336,461, filed on Nov. 14, 2001.
(60) Provisional application No. 60/357,275, filed on Feb. 15, 2002.
(60) Provisional application No. 60/362,858, filed on Mar. 7, 2002.
(60) Provisional application No. 60/376,813, filed on Apr. 29, 2002.
(60) Provisional application No. 60/379,336, filed on May 9, 2002.
(60) Provisional application No. 60/382,309, filed on May 20, 2002.
(60) Provisional application No. 60/393,635, filed on Jul. 3, 2002.
(60) Provisional application No. 60/400,652, filed on Aug. 2, 2002.
(60) Provisional application No. 60/400,218, filed on Jul. 31, 2002.
(60) Provisional application No. 60/400,630, filed on Aug. 2, 2002.
(60) Provisional application No. 60/405,314, filed on Aug. 21, 2002.
(60) Provisional application No. 60/409,296, filed on Sep. 6, 2002.

(51) Int. Cl. C12Q 1/68; G01N 33/53; C12M 1/34

(52) U.S. Cl. 435/6; 435/7.1; 435/287.2

(57) ABSTRACT

Systems and methods of biochemical analysis are provided. A method of performing biochemical assays includes loading a first plurality of reservoirs on a first liquid carrier with a first plurality of compounds, coupling a second carrier with the first carrier, the second carrier having a second plurality of reservoirs configured to couple with the first plurality of reservoirs and containing a second plurality of compounds, transferring at least a portion of the first plurality of compounds to the second plurality of reservoirs, and separating at least one component from the second plurality of reservoirs. A system for biochemical analysis includes a first carrier and a second carrier. The first carrier includes a first substrate and a plurality of reservoirs in the substrate for retaining a first plurality of compounds. The second carrier includes a second substrate and a plurality of projections, each projection having a distal end provided with a receiving feature for receiving a component from the plurality of reservoirs when the first carrier and the second carrier are coupled.

### CROSS-REFERENCE TO RELATED APPLICATIONS

This disclosure claims the benefit of priority to the following U.S. applications: U.S. Application Serial No. 60/336,461, entitled “Single Use XHTS Chip” by Shiping Chen, filed Nov. 14, 2001; U.S. Application Serial No. 60/357,275, entitled “Reagent Metering” by Shiping Chen, filed Feb. 15, 2002; U.S. Application Serial No. 60/362,858, entitled “Method and Apparatus for Picoliter Precision Assays” by Shiping Chen, filed Mar. 7, 2002; U.S. Application Serial No. 60/376,813, entitled “Additional Method and Apparatus for Picoliter Precision Assays” by Shiping Chen, filed Apr. 29, 2002; U.S. Application Serial No. 60/379,336, entitled “Additional Method and Apparatus for Picoliter Precision Assays” by Shiping Chen et al., filed May 9, 2002; U.S. Application Serial No. 60/382,309, entitled “Methods and Apparatus for Heterogeneous and Other Assays” by Shiping Chen et al., filed May 20, 2002; U.S. Application Serial No. 60/393,635, entitled “Additional Methods and Apparatus for Heterogeneous and Other Assays” by Shiping Chen et al., filed Jul. 3, 2002; U.S. Application Serial No. 60/400,652, entitled “Miniaturized Heterogeneous Assay Formats for High Throughput Screening” by Theo Nikiforov, filed Aug. 2, 2002; U.S. Application Serial No. 60/400,218, entitled “Parallel Picoliter Synthesis” by Shiping Chen et al., filed Jul. 31, 2002; U.S. Application Serial No. 60/400,630, entitled “Additional Methods and Apparatus for Heterogeneous and Other Assays” by Theo Nikiforov et al., filed Aug. 2, 2002; U.S. Application Serial No. 60/405,314, entitled “Biochemical Analysis System” by Shiping Chen et al., filed Aug. 21, 2002; and U.S. Application Serial No. 60/409,296, entitled “Biochemical Analysis System with Combinatorial Chemistry Applications” by Shiping Chen et al., filed Sep. 6, 2002. All of the above applications are incorporated by reference herein in their entireties as if fully set forth below for all purposes.

### BACKGROUND OF THE INVENTION

Embodiments of the present invention relate generally to biochemical analysis, and, in particular, relate to methods, devices, and compositions relating to the gauging of the interaction of targets from one or multiple solutions to probes, including the fields of high throughput screening (HTS), proteomics, and polymerase chain reaction (PCR) amplification.

Many biochemical investigations involve performing a set of experiments that mix one or a small number of reagents with individual chemical or biological entities in a large set and readout the results of the reactions. In “High Throughput Screening” (HTS), the reagents can be enzymes and substrates while the entities are a library of chemical compounds. In protein microarray applications, the reagent can be a sample protein mixture while the entities are known as protein probes. In polymerase chain reaction (PCR) applications, the reagent can be a sample DNA mixture while the entities are pre-designated primers.

In all these applications, it may be desirable to perform as many experiments as possible in parallel and to consume as little reagents and biochemical entities as possible in these experiments. Many times, reagents are expensive or can only be purified from natural starting materials with great difficulty and/or expense.

The process of drug discovery is often dependent upon the ability of screening efforts to identify lead compounds with future therapeutic potential. The screening efforts are often described as one of the bottlenecks in the process of drug discovery. One strategy for identifying pharmaceutical lead compounds is to develop an assay that provides appropriate conditions for monitoring the activity of a therapeutic target for a particular disease. This assay is then used to screen large numbers of potential modulators of the therapeutic target in the assay. For example, libraries of chemical compounds can be screened in assays to identify their activity in relation to therapeutic targets and cells.

Biochemical and biological assays are designed to test for activity of chemical entities in a broad range of systems including protein-protein interactions, enzyme catalysis, small molecule-protein binding, and other cellular functions. In “High Throughput Screening” (HTS), these kinds of assays can be used to simultaneously test a large number of chemical entities in order to discover biological or biochemical activities of the chemical entities.

Current high-throughput screening (HTS) technologies are based on microtiter plates (96-, 384-, or 1536-well plate) with most widely established techniques utilizing 96-well microtiter plates. In this format, 96 independent tests are performed simultaneously on a single 8 cm×12 cm plastic plate that contains 96 reaction wells. These wells typically require assay volumes that range from 50 to 500 μl. In addition to the plates, many instruments, materials, pipettes, robotics, plate washers and plate readers are commercially available to fit the 96-well format to a wide range of homogeneous and heterogeneous assays.

To date, efforts to improve HTS have generally focused on miniaturization. By reducing the well size, the number of wells on each plate can be increased in order to provide more parallel testing. Furthermore, by decreasing assay volumes, the amount of reagents is also reduced. Moreover, because more parallel tests can be run with smaller assay volumes, the simultaneous testing of more compounds to find drug candidates can be accelerated. Miniaturization has marginally improved the 96-well technology by providing a 384-well format.

Homogeneous assays are sometimes referred to as “mix-and-read” assays, or “addition-only” assays. Assay formats such as fluorescence polarization, homogeneous time-resolved resonance energy transfer, and homogeneous proximity-based assays, etc., can be used in homogeneous assays. One common feature of all of these assays is that they do not require any separation steps. Rather, these methods allow the determination of the degree of substrate-to-product conversion to be carried out in a homogeneous solution containing both species. These homogeneous assay formats may offer significant advantages in terms of reduced liquid handling needs. Compared to homogeneous assays, heterogeneous assays may provide better signal-to-noise ratios, sensitivities, and requirements for degree of substrate-to-product conversion. In heterogeneous assay formats, at the end of the enzymatic reaction, substrate and product are usually completely separated. Numerous methods have been used to achieve the separation of substrate and product, such as, for example, filter binding, binding to immobilized antibodies, binding to ion exchange of affinity matrices, separations by chromatography, electrophoresis, and others.

### BRIEF SUMMARY OF THE INVENTION

In accordance with embodiments of the present invention, a method of performing biochemical assays includes loading a first plurality of reservoirs on a first liquid carrier with a first plurality of compounds, coupling a second carrier with the first carrier, the second carrier having a second plurality of reservoirs configured to couple with the first plurality of reservoirs and containing a second plurality of compounds, transferring at least a portion of the first plurality of compounds to the second plurality of reservoirs, and separating at least one component from the second plurality of reservoirs.

In accordance with further embodiments of the present invention, a system for biochemical analysis including a first carrier and a second carrier is provided. The first carrier includes a first substrate and a plurality of reservoirs in the substrate for retaining a first plurality of compounds. The second carrier includes a second substrate and a plurality of projections, each projection having a distal end provided with a receiving feature for receiving a component from the plurality of reservoirs when the first carrier and the second carrier are coupled.

In accordance with further embodiments of the present invention, a method of performing biochemical analysis is provided. The method comprises: coupling a first carrier with a second carrier, the first carrier having a plurality of protrusions, each protrusion being provided with a capture component, and the second carrier having a plurality of reservoirs containing a plurality of compounds and being configured such that each of the plurality of reservoirs receives at least one of the protrusions; uncoupling the first carrier from the second carrier; and retrieving at least one target component from the plurality of reservoirs with at least one capture component.

In accordance with further embodiments of the present invention, a biochemical analysis system is provided, comprising: a first carrier having a plurality of projections, each projection having a distal end provided with one reservoir from a first plurality of reservoirs; and a second carrier including a second plurality of reservoirs, each of said second plurality of reservoirs being positioned to receive at least one of the plurality of projections on the first carrier and being configured such that when the second carrier is coupled with the first carrier, a liquid contained within each of the first plurality of reservoirs transfers to a corresponding reservoir in the second plurality of reservoirs.

In accordance with further embodiments, a biochemical analysis system is provided, comprising: a first carrier having a first plurality of reservoirs; a second carrier including a second plurality of reservoirs, each of said second plurality of reservoirs being positioned to correspond to at least one of the first plurality of reservoirs on the first carrier and being configured such that when the second carrier is coupled with the first carrier, a liquid contained within each of the first plurality of reservoirs transfers to a corresponding reservoir in the second plurality of reservoirs; and a loading station, comprising: a plurality of storage vessels; and a delivery device coupled to the plurality of storage vessels for loading a plurality of liquids into each of the first plurality of reservoirs on the first carrier.

In accordance with further embodiments, the biochemical analysis system includes an assay station, comprising: a first stage for retaining the first carrier; a second stage for retaining the second carrier; and a positioning system for positioning the first stage and the second stage to precisely couple the first carrier with the second carrier.

In accordance with further embodiments, a biochemical analysis system is provided, comprising: a first carrier having a first plurality of reservoirs; and a second carrier including a plurality of through-holes, each through-hole in said plurality of through-holes being positioned to correspond to at least one of the first plurality of reservoirs on the first carrier and being configured such that when the second carrier is coupled with the first carrier, a liquid contained within each of the first plurality of reservoirs transfers to a corresponding through-hole in the second carrier; wherein each of said plurality of through-holes contains a capture probe attached to an interior surface of the through-hole, said capture probe being configured to capture a target molecule.

In accordance with further embodiments, a method of performing biochemical assays is provided, comprising: loading a first plurality of reservoirs on a first carrier with a first plurality of compounds, said first carrier having a first plurality of projections, each of the first plurality of reservoirs being provided on one of the first plurality of projections; coupling a second carrier with the first carrier, the second carrier having a second plurality of reservoirs containing a second plurality of compounds and configured to receive at least one of the first plurality of projections; and transferring at least a portion of the first plurality of compounds to the second plurality of reservoirs.

In accordance with further embodiments, a method of combinatorial chemical synthesis is provided. The method comprises: loading a first set of reagents into a first set of reservoirs on a first carrier, said first set of reagents comprising reagents A₁ through Aₓ, wherein the first set of reservoirs contain y number of reservoirs containing each of the reagents A₁ through Aₓ; loading a second set of reagents into a second set of reservoirs on a second carrier, said second set of reagents comprising reagents B₁, through B_{y}, wherein the second set of reservoirs contain x number of reservoirs containing each of the reagents B₁ through B_{y}; and coupling the first carrier and the second carrier to load at least a portion of the second set of reagents into the first set of reservoirs such that each reservoir in the first set of reservoirs contains a unique combination of one of the reagents A₁ through Aₓ and one of the reagents B₁ through B_{y}; wherein x and y are integers greater than one.

Embodiments of the present invention can provide an extremely flexible format for performing complex assays in an assay carrier having a plurality of through-hole wells. Multiple reagents can be individually or universally introduced into the through-holes using multiple metering carriers in series or in parallel. The resulting mixture can then be observed for detectable signals and additional reagents can be introduced, if desired. In addition, the metering carriers and/or the assay carriers can be loaded with compounds in a central location and then shipped to the end user's location, where the assays can be performed.

Other features and aspects of the invention will become apparent from the following detailed description, taken in conjunction with the accompanying drawings which illustrate, by way of example, the features in accordance with embodiments of the invention. The summary is not intended to limit the scope of the invention, which is defined solely by the claims attached hereto.

### BRIEF DESCRIPTION OF THE SEVERAL VIEWS OF THE DRAWING(S)

FIG. 1 shows a multi-carrier screening system.

FIGS. 2a-2b show top and cross-sectional views of a reagent metering carrier.

FIGS. 3a-3f show various embodiments of pillars having interior regions forming reservoirs.

FIGS. 4a-4b show top and cross-sectional views of an assay carrier.

FIG. 5 shows a reagent carrier coupled with an assay carrier.

FIGS. 6a-6d show embodiments of a pillar-through-hole coupling design.

FIG. 7 shows a reagent carrier coupled with an assay carrier.

FIGS. 8a-8b show exemplary capture carriers.

FIGS. 9a-9c show a method of loading a liquid into empty through-holes on an assay carrier.

FIGS. 10a-10d show another method of loading a liquid into empty through-holes on an assay carrier.

FIGS. 11a-11d show embodiments of structured through-holes having multiple chambers.

FIGS. 12a-12e show systems for universal reagent loading.

FIGS. 13a-13c show systems for sealing the top and bottom surfaces of an assay carrier.

FIGS. 14a-14c show methods for compensating for evaporation.

FIGS. 15a-15b show capture molecules for binding biochemical molecules in the assay solution.

FIGS. 16a-16d show capture molecules incorporating magnetic beads.

FIGS. 17a-17b show embodiments of through-hole configurations.

FIGS. 18a-18c show the optical inspection of through-holes and pillars.

FIGS. 19a-19c show a fluorescence-based detection approach incorporating a grating layer on the surface of the pillar tip.

FIG. 20 shows a liquid delivery system

FIG. 21a shows a cross-sectional view of a staging device.

FIG. 21b shows a top view of a staging device having a single through-hole aligned with cavities in a capillary bundle of a liquid delivery system.

FIG. 21c shows a top view of another staging device having multiple through-holes aligned with cavities in a capillary bundle of a liquid delivery system.

FIG. 21d shows a top view of another staging device having high density through-holes aligned with cavities in a capillary bundle of a liquid delivery system.

FIGS. 22a-22c show a process for precision liquid delivery to a staging device.

FIG. 23 shows a process for precision liquid delivery from a staging device to a substrate.

FIG. 24 shows a staging device being loaded.

FIG. 25 shows a cross-sectional and top view of a through-hole.

FIG. 26 shows a screening procedure.

FIGS. 27a-27e show embodiments of structured through-holes.

FIGS. 28a-28c show reagent metering devices.

FIGS. 29a-29c show liquidic features on the top surface of carriers.

FIGS. 30a-30f show a loading process.

FIG. 31 shows a container configuration having parallel loading chambers.

FIGS. 32a-32b show features on the top surface of carriers for isolating different reagents.

FIG. 33 shows another container configuration having parallel loading chambers.

FIG. 34 shows an arrangement of reagents loaded onto a metering carrier.

FIGS. 35a-35b show a multiple reagent loader.

FIGS. 36a-36b show another multiple reagent loader.

FIG. 37 shows an arrangement of reagents loaded onto a metering carrier.

FIGS. 38a-38d show the mixing of reagents.

FIG. 39 shows the mixing of a third reagent.

FIG. 40 shows an exemplary pin carrier.

FIG. 41 shows two pin carriers coupled with an assay carrier.

FIGS. 42a-42b show pins and pin probes.

FIG. 43 shows an exemplary pin set.

FIG. 44 shows a pin probe capturing a target in an assay carrier solution.

FIG. 45 shows a pin probe linked to an addressing component, a target, and a detection component.

FIG. 46 shows an ATP analog for use with embodiments of the present invention.

FIGS. 47a-47e show antibody immobilization via a carbohydrate moiety.

FIGS. 48a-48f show a receptor binding assay under non-equilibrium conditions.

FIGS. 49a-49e show a receptor binding assay within a fiber optic capillary under equilibrium conditions.

FIGS. 50a-50b show a capture chamber on a synthesis assay carrier.

FIGS. 51a-51b show another embodiment of a capture chamber on a synthesis assay carrier.

FIGS. 52a-52e show an arrangement of reagents loaded onto a plurality of carriers for combinatorial chemistry synthesis.

FIGS. 53a-53d show another arrangement of reagents loaded onto a plurality of carriers for combinatorial chemistry synthesis.

FIGS. 54a-54b show another embodiment of a through-hole having a raised surface feature on an end of the through-hole opposite the capture chamber.

FIGS. 55a-55b show another embodiment of a through-hole having a raised surface feature on the same end of the through-hole as the capture chamber.

FIG. 56 shows an embodiment in which four different functional groups are attached to a scaffold to synthesize 200,000 different chemicals.

FIGS. 57a-57d show an embodiment for a high throughput screening assay.

FIG. 58 shows an embodiment in which more than three different reagents are mixed at different times in an assay carrier.

FIGS. 59a-59b show an embodiment for performing cell-based assays.

FIGS. 60a-60d show another embodiment for performing cell-based assays.

FIGS. 61a-61d show another embodiment for performing cell-based assays.

FIGS. 62a-62d show an embodiment having sloped walls.

FIGS. 63a-63c show a combination metering/assay carrier.

FIGS. 64a-64c show an embodiment for serial dilution.

FIG. 65 shows the concentration gradient along an extended through-hole.

FIG. 66 shows a through-hole carrier assembly subjected to a magnetic field.

FIG. 67 shows a through-hole carrier assembly subjected to a voltage.

In the following description, reference is made to the accompanying drawings which form a part thereof, and which illustrate several embodiments of the present invention. It is understood that other embodiments may be utilized and structural and operational changes may be made without departing from the scope of the present invention. The use of the same reference symbols in different drawings indicates similar or identical items.

### DETAILED DESCRIPTION OF THE INVENTION

Certain embodiments of the present invention may achieve significant enhancement in increasing the number of parallel experiments and in decreasing the amount of reagents and biochemical entities consumed. HTS is used herein as an example application to illustrate the functionality of embodiments of the invention disclosed herein. It will be understood that embodiments of the present invention can be applied in a variety of processes and are not limited to only HTS applications.

Embodiments of the invention can be used, e.g., for genomic analysis, to analyze catabolic and anabolic reactions which occur in living systems including enzymatic, binding, signaling and other reactions. Other applicable biochemical systems include model systems which are mimetic of a particular biochemical interaction. Examples of applicable biochemical systems include, for example, receptor-ligand interactions, enzyme-substrate interactions, cellular signaling pathways, transport reactions involving model barrier systems (e.g., cells or membrane fractions) for bioavailability screening, and a variety of other general systems. Cellular or organismal viability or activity may also be screened using embodiments of the present invention, e.g., in toxicology studies. Biological materials which can be assayed include, but are not limited to, cells, cellular fractions (membranes, cytosol preparations, etc.), agonists and antagonists of cell membrane receptors (e.g., cell receptor-ligand interactions such as e.g., transferrin, c-kit, viral receptor ligands (e.g., CD4HIV)), cytokine receptors, chemokine receptors, interleukin receptors, immunoglobulin receptors and antibodies, the cadherein family, the integrin family, the selectin family, and the like.

Other applicable biochemical systems for use with the present invention are described in greater detail in “Methods in Enzymology (Guide to Molecular Cloning Techniques Vol. 152),” edited by Shelby L. Berger et al., Academic Press (November 1997); Joseph Sambrook et al., “Molecular Cloning: A Laboratory Manual,” Cold Spring Harbor Laboratory (3rd ed. 2001); Frederick M. Ausubel, “Current Protocols in Molecular Biology,” Lippincott, Williams & Wilkins (1988); James D. Watson et al., “Molecular Biology of the Gene,” Addison-Wesley Pub. Co. (4th ed. 2001); Bruce Alberts et al., “Molecular Biology of the Cell,” Garland Pub. (4th ed. 2002); “Merck Manual Diagnosis & Therapy,” edited by Mark H. Beers et al., Merck & Co. (17th ed. 1999); “Harrison's Principles of Internal Medicine,” edited by Eugene Braunwald et al., McGraw-Hill Professional (15th ed. 2001); and Benjamin Lewin, “Genes VII,” Oxford University Press (7th ed. 1999).

Aspects described in the following patent applications may apply to various aspects and embodiments of the present invention: PCT Application entitled “Biochemical Analysis System with Combinatorial Chemistry Applications” by Shiping Chen, Yuling Luo, Anthony C. Chen, Theo Nikiforov, Jianming Xiao, and Quan Nguyen, filed on Nov. 14, 2002; U.S. patent Publication Ser. No. 2002/0051979, entitled “Microarray Fabrication Technologies” by Shiping Chen et al., filed Feb. 22, 2001; U.S. patent Publication Ser. No. 2002/0053334, entitled “Microarray Fabrication Techniques and Apparatus” by Shiping Chen et al., filed Feb. 22, 2001; PCT Publication WO 01/62377, entitled “Microarray Fabrication Technologies” by Shiping Chen, filed Feb. 22, 2001; PCT Publication WO 01/62378, entitled “Microarray Fabrication Techniques and Apparatus” by Shiping Chen et al., filed Feb. 22, 2001; U.S. patent Publication Ser. No. 2002/0055111, entitled “Three Dimensional Probe Carriers” by Shiping Chen et al., filed Aug. 24, 2001; PCT Publication WO 02/16651, entitled “Three Dimensional Probe Carriers” by Shiping Chen et al., filed Aug. 24, 2001; U.S. patent Publication Ser. No. 2002/0028160, entitled “Method and Apparatus Based on Bundled Capillaries for High Throughput Screening” by Shiping Chen et al., filed Feb. 22, 2001; U.S. patent application Ser. No. 10/080,274, entitled “Method and Apparatus Based on Bundled Capillaries for High Throughput Screening” by Shiping Chen et al., filed Feb. 19, 2002; PCT Publication WO 02/078834, entitled “Method and Apparatus Based on Bundled Capillaries for High Throughput Screening” by Shiping Chen et al., filed Feb. 19, 2002; and U.S. patent Publication Ser. No. 2001/0055801, entitled “Liquid Arrays” by Shiping Chen et al., filed Feb. 22, 2001. All of the above applications are incorporated by reference herein in their entireties as if fully set forth below for all purposes.

Embodiments of the present invention may be capable of performing a large number of chemical and biochemical reactions in parallel with minute reagent volume. As used herein, reagents that are different from the reagents used in parallel reactions are termed “individual reagents” (IR). For example, when a plurality of reservoirs in a carrier are filed with individual reagents, each of the reservoirs contains a unique reagent, different from the others. The reagents that are used by all the parallel reactions are termed “universal reagents” (UR). Accordingly, when a plurality of reservoirs in a carrier are filed with a universal reagent, all of the reservoirs contain the same reagent.

As used herein, the term “reservoir” refers to a cavity, aperture, through-hole, receptacle, chamber, groove, or region on a surface for holding or containing a liquid. The liquid may be retained in the reservoir by, for example, physical confinement, capillary action, or surface tension.

I. Multi-Carrier Screening System

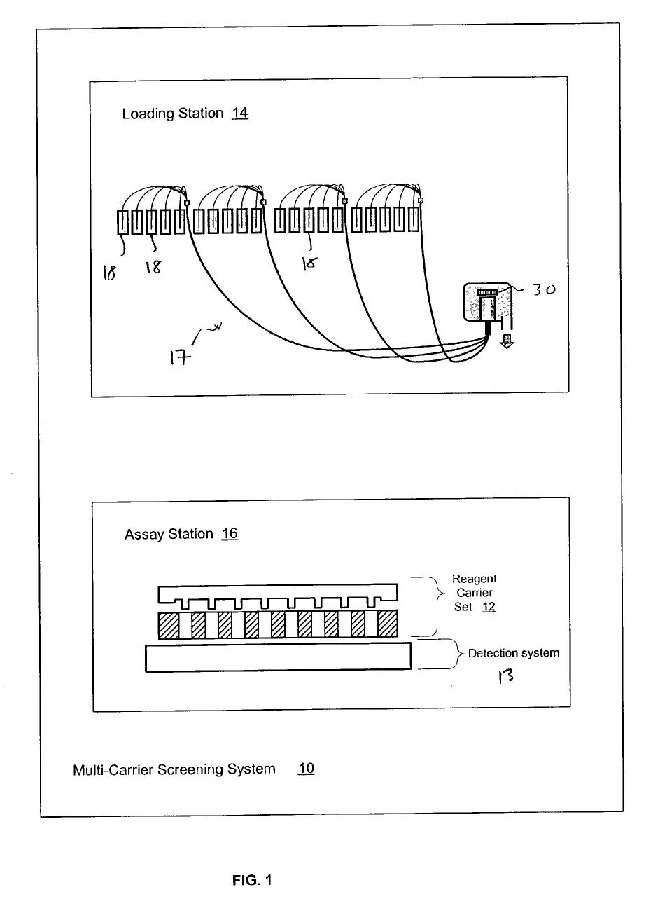

FIG. 1 shows a multi-carrier screening system 10, in accordance with embodiments of the present invention. Screening system 10 may include a a loading station 14 and an assay station 16. The assay station 16 may include a carrier set 12 and a detection system 13.

A Carrier Set

The carrier set is a set of liquid carriers which can be used in biochemical analysis or high throughput screening assays for performing various functions including volume metering, sample storage/shipment, reagent mixing, and separation. The carriers in the carrier set may come in various forms, including a “reagent metering carrier”, an “assay carrier” and a “capture carrier”. All of these carriers can be fabricated using, for example, a combination of DRIE (Deep Reactive Ion Etching) and wafer bonding techniques. Other alternative methods of fabrication include micro-molding, electroplating, Micro EDM (Electrical Discharge Machining), stereolithography and wet etching of wafers. Various fabrication methods which can be utilized to form carriers in accordance with embodiments of the present invention are described in “Fundamentals of Microfabrication: The Science of Miniaturization” by Marc J. Madou, CRC Press (2nd ed. 2002). Materials that can be used for the carriers include, for example, silicon, ceramic, glass, polymer, metal oxide, and other suitable metals, such as stainless steel. Other possible materials are described in “Materials Science and Engineering: An Introduction,” by William D. Callister, Jr., John Wiley & Sons (5th ed. 1999).

1. Reagent Metering Carrier

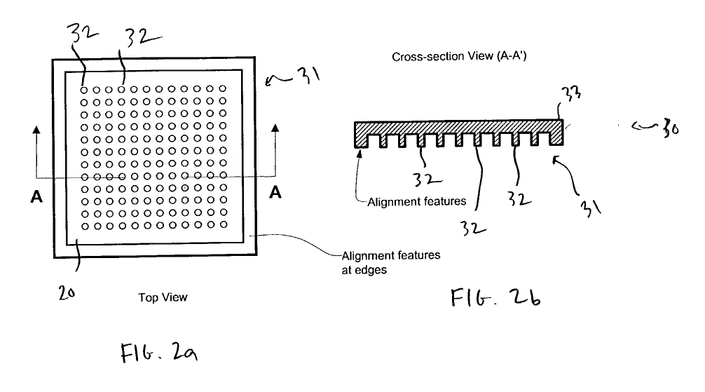

As illustrated in FIG. 2, a reagent metering carrier 30 comprises an array of protruding projections or pillar shaped structures 32 distributed on a substantially flat substrate 33. One or multiple internal cavities can be formed at the tip of each pillar/projection 32.

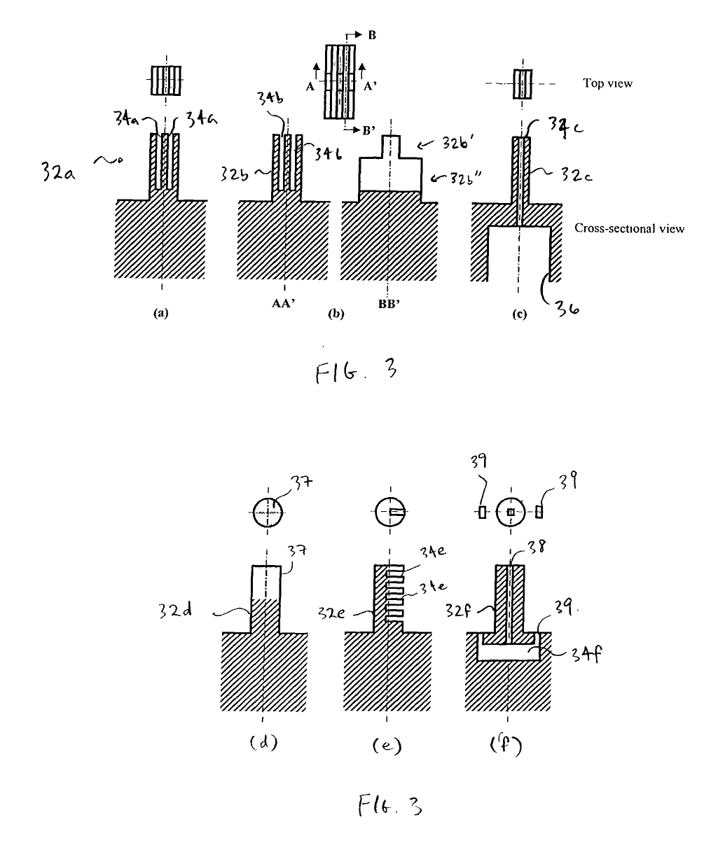

FIGS. 3a-3f illustrate several exemplary configurations for the pillar 32 and cavities 34. In FIG. 3a, the pillar 32a is a simple straight projection having a constant rectangular horizontal cross-section. Two cavities 34a are formed within the pillar 32a as slots extending into the projection 32a. These slots are enclosed by two walls and a bottom formed by the pillar 32a. Two sides and the top of the slots 32a are open to allow liquid to enter and exit the slots as will be described in greater detail below. In one embodiment, the projection 32a comprises glass or silicon and the slots are formed in the projection 32a using conventional microfabrication techniques such as, e.g., DRIE or injection molding. In other embodiments, sacrificial layers can be used to form the slots.

In FIG. 3b, the pillar 32b has an upper portion 32b′ having a horizontal cross-section similar to the cross-section of the pillar 32a in FIG. 2a, and a lower portion 32b″ having an enlarged cross-section. This enlarged lower portion 32b″ enables the cavities 34b in pillar 32b to hold more liquid than the cavities 34a shown in FIG. 3a. FIG. 3c shows a slot-shaped cavity 34c in the pillar 32c which links to a cavity reservoir 36 in the base. This cavity reservoir 36 can be used to supply liquid into cavity 34c.

In FIG. 3d, the cavities are pores formed in a highly porous region 37 of the pillar 32d. The total cavity volume is determined by the solid/pore ratio and the total size of the porous material. In one embodiment, the porous region 37 is formed of porous silicon. This porous region 37 can be fabricated on a silicon substrate using, e.g., electrochemical etching techniques.

In FIG. 3e, the cavities 34e comprise one or more horizontal slots cut into the pillar 32e. In other embodiments, one or more vertical slots can be added to link the interior regions of the cavities 34e together.

In FIG. 3f, the cavity 34f is formed within pillar 32f and around the base of pillar 32f such that one exit 38 of the cavity 34f is provided at the tip of the pillar 32f, and additional exits 39 are located around the base of the pillar 32f. This structure can be fabricated using, for example, a combination of DRIE and wafer bonding techniques.

The inner surfaces of the cavities 34 can be made to be sufficiently hydrophilic such that liquid will be drawn into the cavities 34 by capillary force. The outer surface of each pillar 32 can be made hydrophobic to improve the pillar's ability to draw in liquid. In addition, the geometric configuration of the cavities 34 can be designed such that there is more than one exit out of the pillar 32, so that the air contained within the cavity 34 can easily escape from the cavity 34 as liquid is drawn into the cavity 34.

The reagent metering carrier 30 can use the cavities 34 to meter and store reagent solutions. There are numerous methods for loading solutions into the cavities 34. In one method, the reagent liquids are brought into contact with one of the entrances of the cavities. Capillary forces draw the liquid in to fill the cavity. Then, any excess liquid remaining outside of the cavity can be removed using various methods such as, e.g., blotting and vacuum suction. The reagent can be stored and shipped to users in the cavity 34 in liquid form. Alternatively, the reagent can be frozen or let dry for storage and shipment. Before use, the reagent can be thawed or re-dissolved with a suitable solvent such as, for example, dimethyl sulfoxide (DMSO), ethanol, or water.

In accordance with aspects of the present invention, a reagent metering carrier 30 can be used to draw, store, and deliver precise volumes of liquid. The combined inner volume of these cavities 34 at each pillar 32 can be precisely controlled in accordance with the reagent volume to be used in a desired assay. For example, two vertical slots having dimensions of 10 μm wide, 40 μm long, and 100 μm deep have a combined inner volume capable of drawing and holding a liquid volume of 80 picoliters.

The density of pillars on the metering carrier can be, for example, more than 10 per square cm, or preferably, more than 100 per square cm, or more preferably, more than 1000 per square cm, or even more preferably more than 10,000 per square cm, or even more preferably more than 100,000 per square cm. The volume of the reagent held in the cavities at each pillar can be, for example, less than 1 μl, or preferably less than 10 nl, or preferably less than 1 nl, or more preferably less than 100 pl, or more preferably less than 10 pl, or more preferably less than 1 pl.

2. Assay Carrier

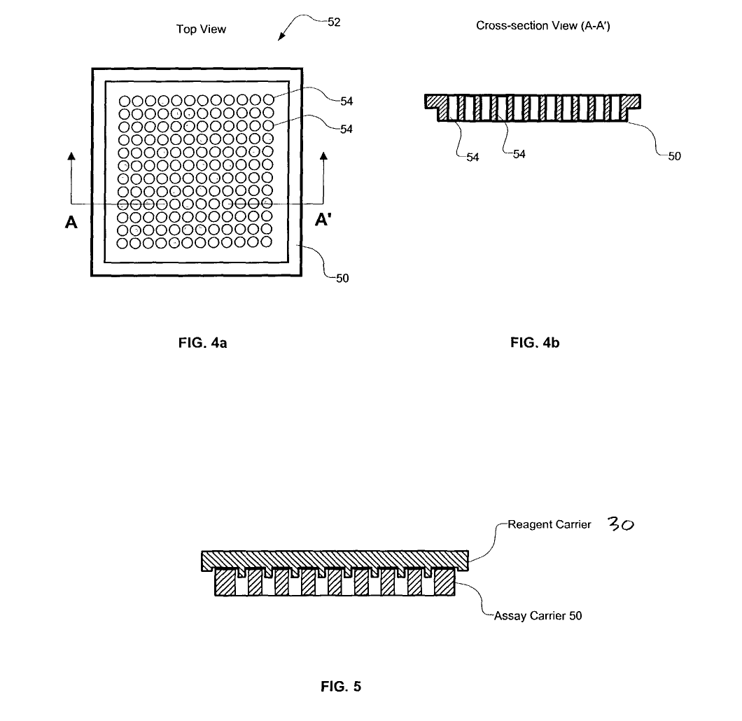

The assay carrier can serve as a platform for reagent mixing, metering, and readout. As shown in FIG. 4a-4b, an assay carrier 50 may comprise a substrate having an array 52 of reservoirs. These reservoirs can be, for example, wells, through-holes, or virtual wells which confine a volume of fluid on a region of the surface using surface tensions. In FIGS. 4a-4b, the reservoirs are formed as through-holes 54 having the same spatial pattern and pitch as that of the pillar array 31 on the corresponding reagent metering carrier 30, so that when the metering carrier 30 and the assay carrier 50 are coupled, as shown in FIG. 5, each of the pillars 32 in the metering carrier 30 is aligned with and inserted into a corresponding through-hole 54 and any fluid contained in the cavities of the metering carrier 30 is in fluid communication with the fluid contained in corresponding through-holes 54 in the assay carrier 50.

In some embodiments, the assay carrier 50 may have a greater number of through-holes 54 than the number of pillars 32 provided on the metering carrier 30. Thus, when the metering carrier 30 is coupled with the assay carrier 50, the pillars 32 of the metering carrier 30 couple with only a subset of the total number of through-holes 54 in the assay carrier 50. In other embodiments, there may be a greater number of pillars 32 than through-holes 54 such that more than one pillar 32 may be inserted into each through-hole 54.

In various embodiments, each of the through-holes 54 can have, for example, a diameter of approximately less than approximately 2000 μm, less than approximately 1000 μm, less than approximately 500 μm, less than approximately 100 μm, or from approximately 1 μm to approximately 10 μm. The pitch of through-holes 54 can range, for example, from approximately 1.2 μm to approximately 2200 μm. The through-holes 54 can have any type of horizontal cross-sectional shape, including, for example, circular, elliptical, square, or rectangular, and can have any of vertical cross-sectional shape, including, for example, rectangular or tapered. In other embodiments, the horizontal ad vertical cross-sectioned shapes are irregular. The pillars 32 can have, for example, a diameter of less than approximately 1000 μm, less than approximately 500 μm, less than approximately 100 μm, or from approximately 5 μm to approximately 10 μm. The pillars 32 can have, for example, a height of less than approximately 5000 μm, less than approximately 2500 μm, less than approximately 1000 μm, less than approximately 500 μm, less than approximately 100 μm, or from approximately 10 μm to approximately 50 μm. Other dimensions and shapes are possible.

In some embodiments, either or both of the metering carrier 30 and the assay carrier 50 may be provided with alignment features to assist in precisely aligning the two carriers. These alignment features may be, for example, ridges, slots, bolts, protrusions, or other alignment mechanisms as would be understood by one of ordinary skill in the art. In FIG. 7, alignment feature 71 on the metering carrier 30 can be used with alignment feature 72 on the assay carrier 50 to align the two carriers 30 and 50. In other embodiments, the various carriers can be precisely aligned using external positioning and alignment systems.

In some embodiments, the inner surfaces of the through-holes 54 are hydrophilic while the external top and bottom surfaces of the assay carrier 50 are hydrophobic. In addition, the interior of each through-hole 54 on the assay carrier 50 can be formed much larger than the portion of the pillar 32 on the metering carrier 30 that is inserted into the through-hole 54 when the carriers 30 and 50 are coupled. The through-hole 54 can be larger by a factor of at least 5, or preferably by a factor of at least 10, more preferably at least 50, more preferably at least 100, more preferably at least 500, even more preferably at least 1000, even more preferably at least 10,000. For example, if a pillar 32 on the metering carrier 30 has a dimension of 40 μm×40 μm×120 μm and 80% of the pillar 32 enters the through-hole 54 in the assay carrier 50, the pillar 32 takes up 152 pl of volume in the through-hole's interior. A through-hole 54 in the assay carrier 50 can have a diameter of 100 μm and a length of 650 μm, resulting a volume of 5.1 nl, which is 33 times larger than the volume taken up by the pillar 32 entering the through-hole 54.

In some embodiments, the interior volume of the through-hole 54 in the assay carrier 50 can be designed to be at a specific ratio to that of the cavities 34 at each pillar 32. The ratio can be at least 5, or preferably at least 10, more preferably at least 50, more preferably at least 100, even more preferably at least 500, even more preferably 1,000, or more preferably at least 10,000. For example, in one embodiment the cavity of a pillar on the metering carrier is a vertical slot measuring 12 μm wide, 40 μm long and 100 μm deep with an inner volume of 48 pl. A through-hole in the assay carrier with a 100 μm diameter and a 650 μm length has an inner volume of 5.1 nl. In this example, the interior volume ratio of the through-hole in the assay carrier and the pillar cavity on the metering carrier is more than 100.

When a pillar 32 of the metering carrier 30 is inserted into the through-hole 54 of the assay carrier 50, it displaces a certain volume of liquid in the through-hole 54. When the volume of the pillar 32 that enters the through-hole 54 is significantly smaller than that of the interior volume of the through-hole 54, the displaced liquid can still be contained within the through-hole. When the pillar volume becomes larger, however, the displaced liquid may emerge from the interior of the through-hole 54 and contact liquid emerging from an adjacent through-hole 54. In such a situation, it may be desirable to prevent liquid cross-talk between different through-holes 54.

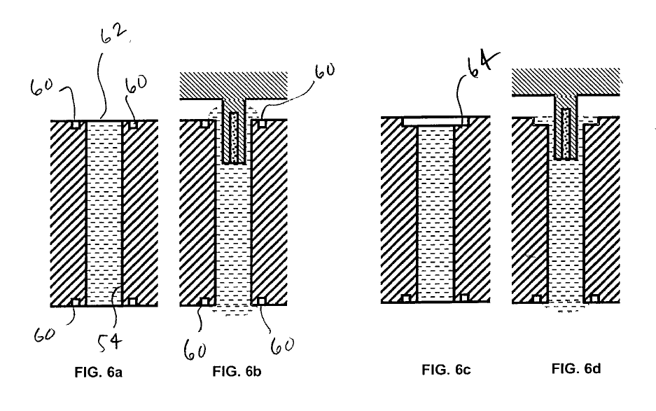

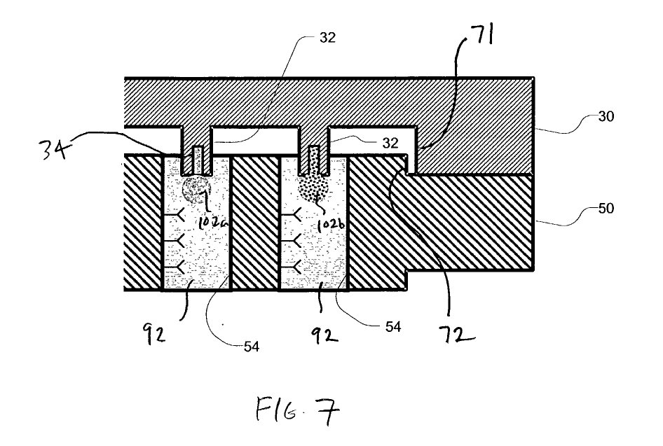

FIGS. 6a-6b and 6c-6d illustrate exemplary configurations for cross-talk prevention. In FIGS. 6a-6b, a trench 60 is formed around the opening of each through-hole 54. As shown in FIG. 6a, the liquid 62 contained within through-hole 54 does not protrude beyond the planes formed by the top and bottom surfaces of the assay carrier 50. When the pillar 32 is inserted into the through-hole 54, as shown in FIG. 6b, the displaced volume of liquid extends beyond the top and bottom surfaces. The trenches 60 formed around the openings of the through-holes 54 provide a physical barrier to retain the liquid 62, thereby preventing cross-talk. In FIGS. 6c-6d, an enlarged section 64 is formed at one exit of the through-hole 54. This enlarged section 64 can be used to retain the displaced liquid. In various embodiments, the trenches 60 and enlarged sections 64 can be used alone or in combination. The trenches 60 can be replaced by annular hydrophobic regions surrounding the opening of the through-holes 54 to similarly prevent cross-talk.

3. Capture Carrier

In accordance with another aspect of the present invention, a capture carrier is provided for capturing and extracting a particular type of molecule out of an array of assay solutions, such as, for example, the assay solution contained within the through-hole 54. This may be performed, for example, to capture the product of a biochemical reaction or the substrate molecules that did not undertake a reaction as part of a heterogeneous assay.

The basic configuration of a capture carrier can be similar to that of a metering carrier, such as those illustrated in FIGS. 3a-3c. The capture carrier may comprise an array of pillars on a substrate. The pitch and spatial pattern of the pillar array can match that of the through-hole array on the assay carrier. Certain surface areas of the pillars or cavities in the pillars can be coated with “capture molecules” that have high affinity with the target molecules to be captured from the solution contained within the through-hole. These “capture molecules” can be, for example, streptavidin, substrate- or product-specific antibodies, polyionic polymers, metal-chelating groups with chelated metal ions, oligonucleotides, other nucleic acids, antibodies, streptavidin, and others.

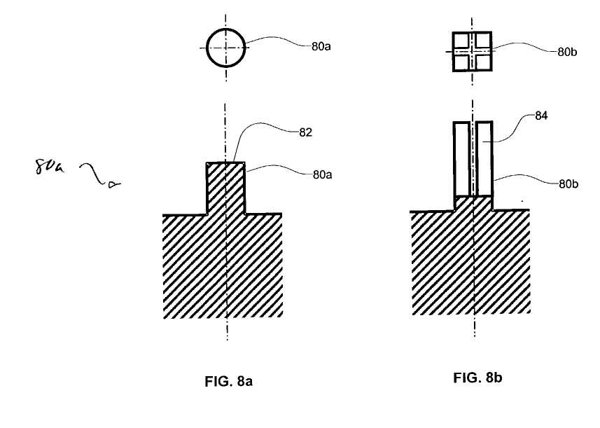

FIGS. 8a-8b show two examples of capture carriers 80. FIG. 8a shows a capture projection 80a optimized for “capture and detect” and FIG. 8b shows a capture projection 80b optimized for “capture and release”. When the “capture and detect” projection 80a is used, signal producing molecules are captured on the capture projection 80a and the capture carrier can then be used as the platform for optical read out. In FIG. 8a, the tip portion 82 of the capture projection 80a is coated with capture molecules to optimize for capture density and detection. After the target molecule is captured onto the tip portion 82, the presence of the target molecule can be optically detected directly from the capture carrier.

When the “capture and release” carrier is used, the signal producing molecules are first captured on the pillars 80b. Then the pillars 80b are extracted from the through-holes 54 of the assay carrier 50 and the molecules captured on the pillars 80b are released back into a solution for additional assaying steps before read out. On the pillars 80b of a “capture and release” carrier, an example of which is depicted in FIG. 8b, the surface to volume ratio of the pillar can be maximized by building small or narrow cavities 84 in the pillar 80b. All the inner surfaces of the pillar 80b can be coated with capture molecules to increase the total amount of captured target molecules. The pillar configuration of the “capture and release” carrier can be similar to that of the pillars 32 on the metering carrier 30, as shown in FIGS. 3a-3c. However, the “capture and release” carrier 80b can be configured to maximize internal surface areas of the inner cavities 84. The total liquid volume that can be contained in the capture pillar 80b can range, for example, from 0.1% to 100% of that in the through-hole 54 of the assay carrier 50.

In various embodiments, the illustrated capture projections 80a-80b can be used for either “capture and detect” and/or “capture and release.”

4. Combination Metering/Assay Carrier

FIG. 63 illustrates an embodiment in which the metering and assay wells are provided on the same carrier. In FIG. 63a, a carrier 630a includes an upper portion having a pillar 631a with a slot 632a. This slot 632a is connected to an assay well 633a. The bottom portion of the assay well 633a can be sealed with a film or cover 634a formed of, for example, a polymer or glass. FIGS. 63b-63c show alternative embodiments in which carriers 630b and 630c are provided with pillars 631b and 631c.

The carriers 630 can be used to first load and store compounds, and then be used to perform the loading of a reagent, followed by the mixing, incubation, and signal detection from the well 633. The individual reagents can be loaded onto the carrier 630 by inserting the pillars 631 into the fluid reagent. The reagent is then drawn into the metering slot 632a, but does not flow into the assay well 633 because the seal 634 creates an air pressure within the assay well 633, inhibiting the introduction of fluid into the well 633. Therefore, the reagent is retained within the metering slot 632.

The carrier 630 can then be stored and shipped to the end user with the reagent contained within the metering slot 632 being stored as a liquid, frozen, or in solid dehydrated form. When the carrier 630 is ready to be used in an assay, the reagent is thawed or rehydrated, as described above. A universal reagent may be introduced into the metering slot 632 by either flooding the top of the carrier 630 or by inserting the pillars 631 into the UR. The seal 634 is then removed or punctured, thereby releasing the air pressure within the well 633 and allowing the UR to be drawn into the assay well 633 by capillary action. The individual reagent and universal reagent can then flow into the well 633 and mix.

In some embodiments, the reagents stored in the metering slots 632 can be provided in highly concentrated form to compensate for the difference in volume of the metering slot 632 and the assay well 633.

B. Stations

In accordance with other aspects of the present invention, various stations can be provided for loading components into the various carriers and performing various processes using the carriers and loaded components.

1. Loading Station

A loading station can be used to transfer individual reagents from macro-scale containers, such as the wells of standard microtiter plates, into different pillars of the reagent metering carrier or into the through-holes of an assay carrier. As illustrated in FIG. 1, the loading station 14 can comprise a bundle of flexible capillaries 17. One end of the bundle can be kept loose while the other end is integrated together. The loose ends of the capillary bundle can be inserted into individual storage vessels 18 containing different reagents. The capillary facets in the integrated end of the bundle form a matrix. The spatial pattern and pitch of the matrix can be designed to match that of the pillars 32 in the metering carrier 30. During reagent transfer, a vacuum can be applied at the integrated end of the bundle to draw IRs from their original containers 18 towards the tips of each individual capillary at the integrated end, as depicted in FIG. 1. Alternatively, a positive pressure can be applied to the loose end of the bundle 17 to drive IRs to the integrated end. In either case, a droplet of IR forms at the facet of each capillary. The pillar tips on the carrier 30 are aligned to the corresponding capillary facets in the bundle and come into contact with the IR droplet. Capillary force can then draw the IRs into the cavities 34 in each pillar 32. After the interiors of the cavities 34 are filled, the metering carrier 30 separates with the capillary bundle 17 and any excess liquid can be removed from the outer surfaces of the pillars 32 by blotting or vacuum suction.

Exemplary loading stations capable of delivering a very large number of small quantities of different liquids in parallel are described in greater detail in U.S. patent Publication Ser. No. 2002/0051979, entitled “Microarray Fabrication Techniques and Apparatus”, and U.S. patent Publication Ser. No. 2001/0053334, also entitled “Microarray Fabrication Techniques and Apparatus”, each of which is incorporated by reference in its entirety herein.

2. Assay Station

The assay station 16 can be a relatively small, preferably desktop sized, instrument, which accepts the IRs preloaded in one or multiple metering carriers, the assay carrier, and universal reagents. The assay station 16 can perform parallel assaying on the carriers in a highly automated fashion. The assay station 16 may further be configured to read and report the assay results. The assay station 16 can be a stand-alone device separate from the loading station 14, or can be provided as part of a larger screening system 10 including a loading station 14.

C. Processes

Various assays can be performed on this platform using a combination one or more of the following steps:

1. Loading a Universal Reagent (UR) into reservoirs on an assay carrier;

2. Loading individual reagents (IR) into filled reservoirs on a carrier;

3. Loading UR into filled reservoirs;

4. Loading UR into a metering carrier

5. Incubation;

6. Washing through-holes on the assay carrier;

7. Extracting molecules from filled reservoirs;

8. Washing the capture carrier;

9. Releasing selected molecules from the capture carrier into reservoirs on the assay carrier;

10. Signal amplification of attached molecules;

11. Signal detection.

1. Loading UR into Reservoirs

A number of methods can be employed to load a single liquid (e.g., a “universal reagent” or “UR”) into the through-holes 54 on an assay carrier 50. In some cases, the through-hole 54 may already be partially filled with a liquid or may have certain substances attached to the inner walls of the through-holes 54.

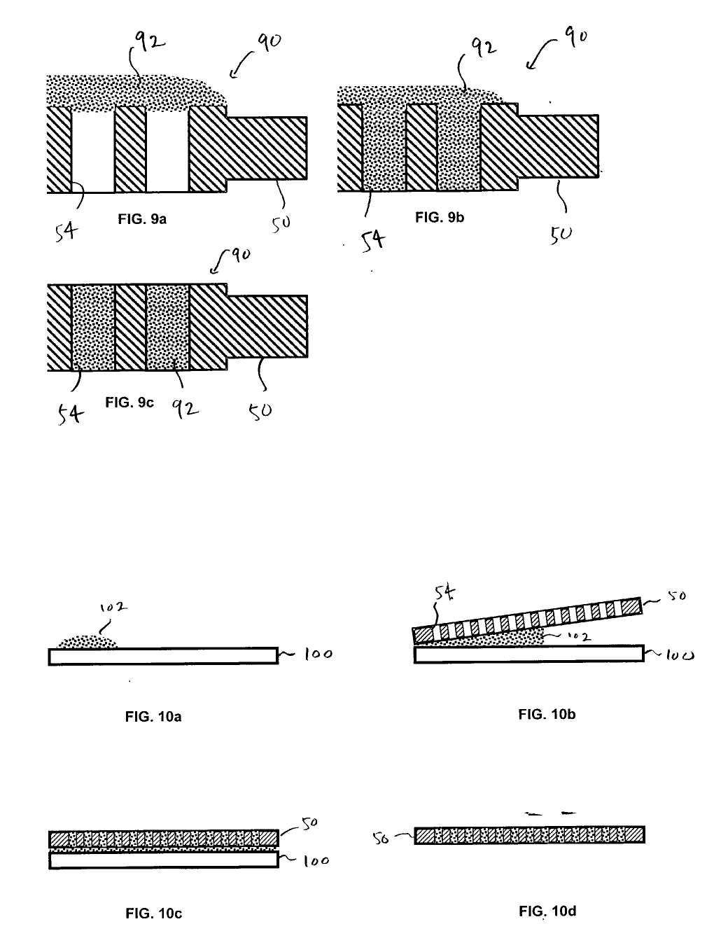

A first method of loading a liquid into empty through-holes 54 on an assay carrier 50 is illustrated in FIGS. 9a-9c. In FIG. 9a, the top surface 90 of the assay carrier 50 is flooded with the liquid 92. In FIG. 9b, the liquid 92 is drawn into the through-holes 54 via, for example, capillary action. Next, the excess liquid on the top surface 90 can be removed by tilting or spinning the carrier 50, wiping, blotting or using vacuum suction, as shown in FIG. 9c.

In some circumstances, a certain minimum volume of UR liquid 92 is used to flood the entire carrier surface 90 and this minimum volume may be more than that required to fill the through-holes 54. A number of methods can be used to provide uniform liquid coverage while minimizing the total liquid volume used to fill the through-holes. For example, a wiper can be used to spread liquid 92 across the surface 90. Alternatively, the assay carrier 50 can be spun at a certain rotation rate while the UR is dropped at the center of the top surface 90.

A second liquid loading method, as illustrated in FIGS. 10a-10d, involves depositing the liquid 102 on a flat substrate 100 and then placing the assay carrier 50 on top of the deposited liquid 102, as shown in FIG. 10b. The pressure of the assay carrier 50 on the deposited liquid 102 forces the droplet to spread across the assay carrier 50 and be drawn into the through-holes 54, as shown in FIG. 10c. Finally, any excess liquid can be removed from the bottom surface of the assay carrier 50, as shown in FIG. 10d.

Alternative methods for filling the assay carrier 50 are described below in the section entitled “ADDITIONAL LOADING METHODS”.

2. Loading IR into Filled Reservoirs

In accordance with aspects of the present invention, methods are provided for loading liquids into the through-holes 54 in the assay carrier 50, when the through-holes 54 have already been filed with a first liquid. In particular, a plurality of individual reagents (“IR”) can be loaded into through-holes 54 already filled with a universal reagent (“UR”).

To load the IR into the filled through-holes 54, the IR is first loaded into reservoirs 34 provided on the projections 32 of a reagent metering carrier 30 using, for example, the loading station 14 as described above. In some embodiments, the loading is conducted at a central location where the loaded metering carrier 30 can be either dried, frozen, or otherwise prepared for shipment to a user. When a user receives a frozen metering carrier 30, the metering carrier 30 can be thawed in the assay station 18. If a user receives a metering carrier 30 containing dried IR, a suitable buffer can be loaded into the slots to re-dissolve the IR.

Next, the pillar array 31 on the metering carrier 30 is coupled with the through-hole array 52 on the assay carrier 50 by bringing the two carriers 30 and 50 together with each pillar 32 being inserted into a corresponding through-hole 54, as illustrated in FIG. 7. The IR solution 102 stored in each reservoir 34 mixes with the liquid 92 already contained in each through-hole 54. The mixing can occur primarily through diffusion. Small motion, such as relative motion between the metering carrier 30 and the assay carrier 50, can enhance the mixing. Such motion can also be achieved through vibration or ultrasound.

The pillar 32 and through-hole 54 remain together until mixing is complete. Then, the metering carrier 30 can be uncoupled from the assay carrier 50. Because the pillars 32 are significantly smaller in volume than that of the interior volume of the through-holes 54 and because the outer surface of the pillars 32 can be made highly hydrophobic, the removal of the pillars 32 from the through-holes 54 will not bring out a significant amount of liquid from the through-hole 54.

3. Loading UR into Filled Reservoirs

Various methods can be used for loading UR into through-holes 54 that already contain liquid.

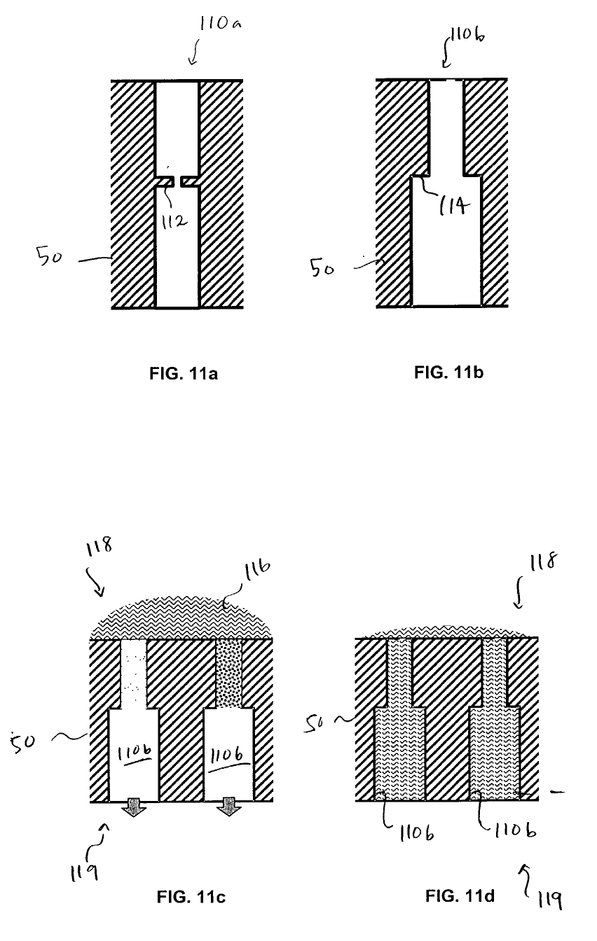

In a first approach, structured through-holes on the assay carrier 50 are used. FIGS. 11a-11d show structured through-holes 110 comprising multiple chambers or “sections” along their length. In such structured through-holes 110, abrupt geometry changes generate microliquidic valves between chambers. In FIG. 11a, the geometry change is provided by a narrowed portion 112 located partway through the through-hole 110a. In FIG. 11b, the geometry change is provided by an expanded portion 114.

As illustrated in FIG. 11c, additional UR 116 can be applied to the assay carrier 50 by flooding a first surface 118 having an opening to the through-holes 110b. A short negative pressure pulse can be applied to the opposite surface 119 to penetrate the microliquidic valves. The new UR 116 is drawn into the through-hole 110b by capillary action and mixes with the pre-existing solution in the through-hole 110b. The amount of new UR 116 equals the interior volume of the previously empty chamber that the liquid just broke into. These microliquidic valves can also be broken using other disturbances, such as acoustic or ultrasonic vibration.

Another method for loading new liquid into a through-hole 54 already containing liquid is by using a metering reagent carrier 30 to load the new liquid. For example, a highly concentrated UR solution can be loaded into the reservoirs 34 on the pillars 32 of a metering carrier 30. The pillars 32 can be inserted into corresponding through-holes 54 on the assay carrier 50 in the same way as loading IR into filled assay through-holes 54 described above. However, in the example described above with respect to the IR loading, a different compound may be contained in each pillar 32 of the metering carrier 30. For UR loading, the same compound is contained in all of the pillars 32.

4. Loading UR into a Metering Carrier

In some embodiments, the IR loading is conducted at the loading station 14, as described above, while the loading of the UR into the pillars 32 of the metering carrier 30 is conducted at the assay station 16. The loading of a single UR liquid into all of the pillars 32 of a metering carrier 30 can be performed in a number of ways.

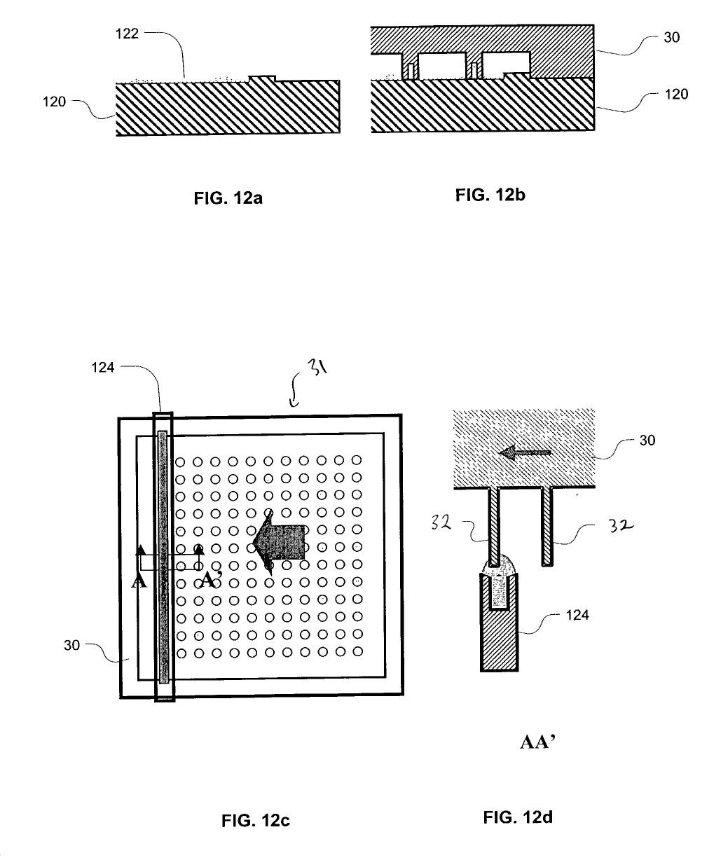

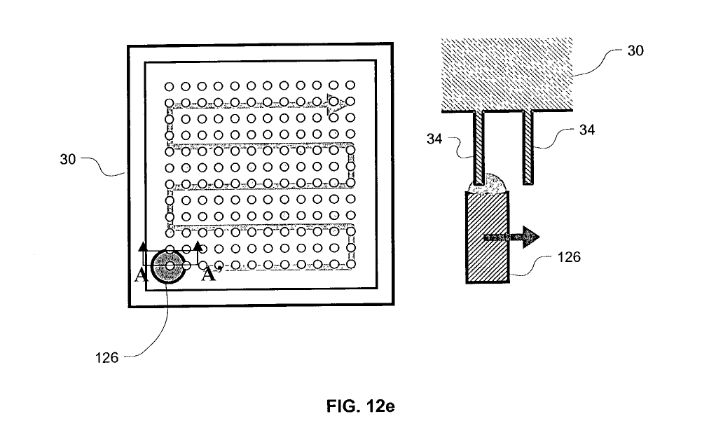

In accordance with a first approach illustrated in FIGS. 12a-12b, the UR is deposited and spread on a flat substrate 120 by, for example, spinning and wiping to generate a liquid film 122. The pillars 32 on the metering carrier 30 are placed in contact with the film 122, thereby drawing the UR into the slots 34.

In accordance with a second approach illustrated in FIGS. 12c-12d, the UR liquid is provided on a loading bar 124 having approximately the same length as that of the pillar array 31 on the metering carrier 30. A long and hydrophilic slot can be formed along the length of the bar 124, which can help to spread the UR liquid uniformly along the entire length of the bar 124. The bar 124 can be overloaded, as shown in FIG. 12d, so that the UR liquid rises above the top surface of the bar and is held in place by surface tension. The metering carrier 30 is brought close to the top surface of the loading bar 124 and, in a relative motion, the bar 124 is drawn across the entire pillar array 31. In this process, each pillar 32 penetrates the liquid surface, thereby drawing up the UR liquid held in the loading bar 124 into the reservoirs 34.

In accordance with a third approach illustrated in FIG. 12e, the loading bar 124 in the preceding approach is reduced to a loading pin 126. The loading pin 126 having a quantity of UR protruding from its top surface is sequentially drawn across each of the pillars 32 in the pillar array 31 on the metering carrier 30 to sequentially let each pillar 32 penetrate the liquid surface and draw up UR liquid.

In accordance with a fourth approach, the UR liquid is loaded into an empty assay carrier 50 having the same pattern and pitch as the pillars 32 on the metering carrier 30 in the same way as loading a empty assay carrier 50 described above. The empty pillars 32 of the metering carrier 30 can be inserted into the through-holes 54 to draw up the UR liquid.

After loading the UR liquid into the reservoirs 34 of the pillars 32 of the metering carrier 30, any excess liquid adhering to the outer surface of the pillars 32 can be removed by blotting or vacuum suction. Specifically, blotting can be achieved by simply tapping the pillars 32 on a flat substrate, such as glass.

5. Incubation

During incubation, the assay carrier 50 can be held under a certain temperature or cycled within a specific temperature range for a certain period of time. Because the liquid stored inside the through-holes 54 of the assay carrier 50 can be in very small volumes, evaporation may become a serious issue. A number of methods can be used either alone or in combination to reduce or eliminate the effects of evaporation.

A first method is to keep the incubation chamber and any other locations within the assay station 16 where the assay carrier 50 and metering carrier 30 are staging at a very high humidity, preferably above 90% RH.

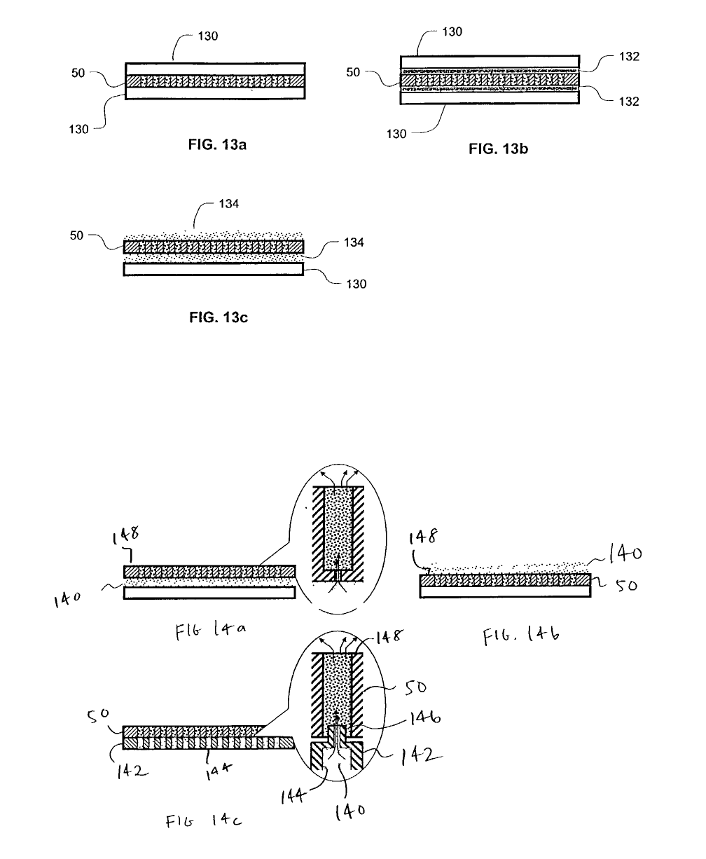

A second method, as illustrated in FIGS. 13a-13c, is to seal the through-holes 54 on the top and bottom surfaces using suitable “lids”, which can be formed by a solid substrate 130 (FIG. 13a), a suitable gasket material 132 (PDMS, for example) on a solid substrate 130 (FIG. 13b), or a suitable non-evaporative liquid 134, such as mineral oil (FIG. 13c). In other embodiments, a reagent carrier can be used to seal the through-holes 54.

A third method is to compensate for the fluid lost in evaporation. In an approach illustrated in FIG. 14a, the assay carrier 50 floats on a liquid layer 140 of bulk buffer or water. When liquid evaporates out of the through-holes 54 from the top surface 148 of the assay carrier 50, capillary forces draw extra buffer 140 into the through-holes 54 to compensate for the loss. When the evaporation rate is sufficiently high, the flow at the bottom opening of the through-hole is always upward, which prevents biomolecules from diffusing out through the bottom of the through-holes 54 into the liquid layer 140. In some embodiments, the through-holes 54 have a reduced cross-section at the opening that contacts the compensating buffer 140. This reduced opening size can increase the upward flow rate at the opening and reduce the chances of diffusion into the liquid layer 140.

In FIG. 14b, the compensating buffer 140 is applied to the top surface 148 of the assay carrier 50 while the evaporation, if any, occurs from the bottom opening of the through-holes 54. The operation of this embodiment is similar to the operation of described above with respect to FIG. 14a.

In FIG. 14c, a compensation carrier 142 having through-holes 144 filled with water or buffer 140 is used for evaporation compensation. Each through-hole 144 has a nozzle or pillar 146 which can be aligned and inserted into a corresponding through-hole 54 on the assay carrier 50 to form a liquidic link. When liquid evaporates from the upper surface 148 of the assay carrier 50, the capillary force draws liquid 140 from the compensation carrier 142.

In various embodiments of the present invention, different solvents and buffers can be used, such as, for example, methanol, ethanol, dimethyl sulfoxide, water, ammonia, and sulfur dioxide.

6. Washing Reservoirs

An aspect of a heterogeneous assay is the separation of one or multiple types of molecules from the remaining molecules in an assay solution in order to improve the signal to noise ratio of the detection. This separation can be achieved by removing non-signal producing molecules from the assay solution or by capturing and then extracting signal producing molecules from the assay solution. Steps 6-11 described below can be relevant to heterogeneous assays.

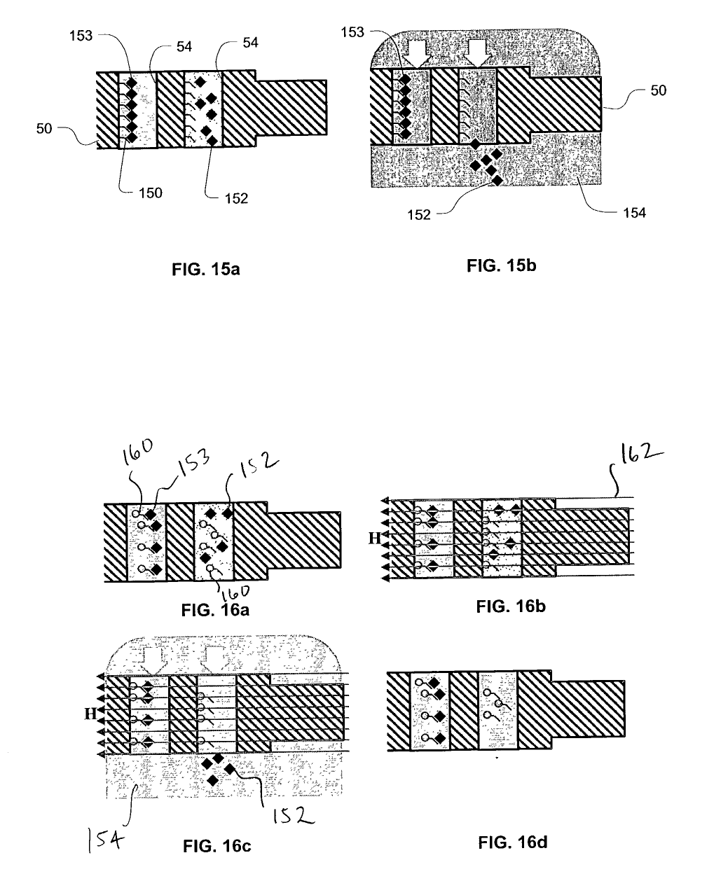

In one specific method of performing heterogeneous assays, the inner wall of the through-hole 54 is coated with special capture molecules 150 that selectively bind biochemical molecules 153 in the assay solution, as shown in FIGS. 15a-15b. These “capture molecules” can be, for example, streptavidin, substrate- or product-specific antibodies, polyionic polymers, metal-chelating groups with chelated metal ions and others.

As shown in FIGS. 15a-15b, the through-holes 54 can be washed by flooding a large amount of suitable washing liquid 154 on one surface of the assay carrier 50 and generating a pressure differential between the two surfaces of the carrier. The washing liquid 154 will flow through the through-holes 54 in the same direction, which washes away any unbound molecules or cells 152. The flow direction can be alternated and/or an ultrasound can be applied to achieve a more vigorous washing.

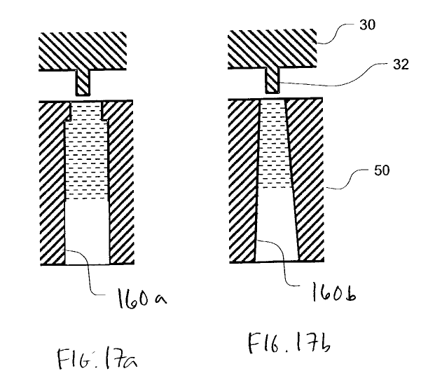

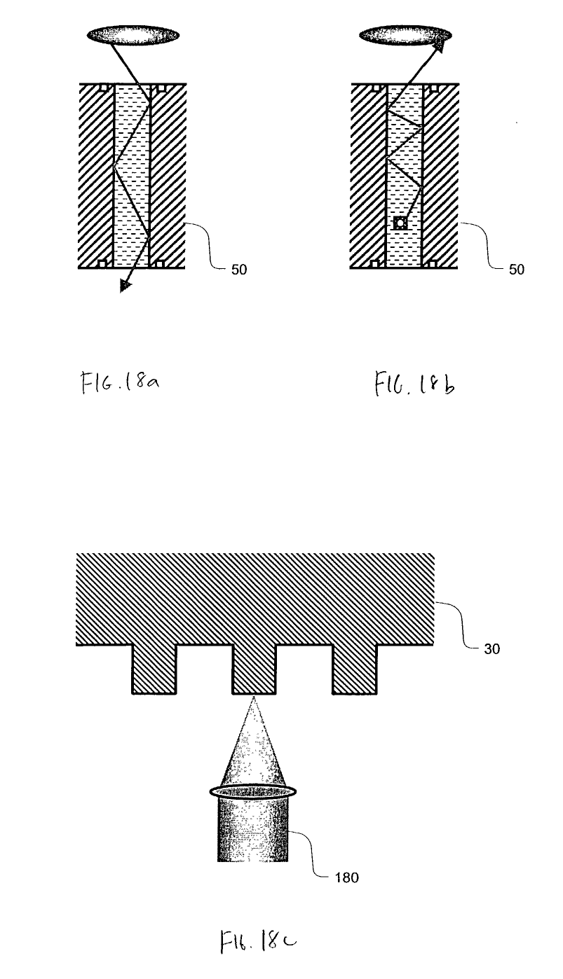

As illustrated in FIGS. 16a-16d, the biochemical molecules or cells 153 can be bound on magnetic beads 160 using methods widely known in by those of ordinary skill in the art. For example, the magnetic beads 160 can be coated with a capture molecule, which is then bound to the desired biochemical molecule or cell. Before washing, an external magnetic field 162 is activated which forces the beads 160 to attach to the side walls of the through-holes 54, as shown in FIG. 16b. While the magnetic field 162 is being applied, the unbound substance 152 can be washed away by driving a suitable washing liquid 154 through the through-holes 54 using, for example, differential pressure, as shown in FIG. 16c. Deactivation of the magnetic field 162 causes the beads 160 and attached biomolecules 153 to be re-suspended in liquid, as shown in FIG. 16d.

7. Extracting Molecules from Filled Reservoirs

Certain types of molecules can be captured and extracted from assay solutions in the through-holes 54 using the “capture carrier” described above with respect to FIGS. 8a-8b. The pillars 80 on the capture carrier can be inserted into the through-holes 54 filled with assay solutions after the biochemical reactions. Specific types of molecules in the assay solution, which could be the product of the reaction (product) or the molecules that are not reacting (substrate), will bind with the corresponding capture reagents immobilized on the surface of the pillars 80. Examples of capture reagents that can be used include streptavidin, substrate- or product-specific antibodies, polyionic polymers, metal-chelating groups with chelated metal ions, nucleic acids and their synthetic analogs, peptides, and others. By removing the capture carrier from the assay carrier 50, specific molecules can be extracted from the assay solutions.

In many steps described above (Steps 2, 3, 7) and in Step 9 described below, the pillar on the metering or capture carrier contacts the liquid inside the through-hole. However, the through-hole may sometimes become less than fully filled due to evaporation or other liquid loss causes. To ensure that the reservoirs 34 on the pillars 32 achieve an effective liquidic contact with the assay solution contained within the through-holes 54, the pillars 32 can be made to be relatively long. Alternatively, the configuration of the through-holes 54 may be specially designed to achieve this goal.

FIGS. 17a-17b show two specific examples of such through-hole configurations. In FIG. 17a, the cross-section of the through-hole 160a near the exit where the pillar 32 is inserted is made to be slightly smaller than the rest of the through-hole 160a. In FIG. 17b, the through-hole 160b is tapered with a smaller end at the inserting exit. In both configurations, the capillary force near the exit where the pillar is inserted is larger than that at the opposite end. In this way, the liquid is preferentially drawn towards the narrowed exit portion of the through-hole 160.

More importantly, this mechanism can be used to “drive” molecules in the liquid contained within the through-hole 160 toward the pillar 32. After the pillar 32 of the capture carrier 30 is inserted into the through-hole 160, a controlled evaporation can be induced by introducing the entire capture/assay carrier stack to an environment with a suitable humidity. The evaporation reduces the solvent volume in the through-hole and concentrates the molecules near the pillar 32. This can drive the binding of selected molecules to the pillar 32 on the capture carrier 30 and increase the amount of molecules to be captured by the capture carrier 30.

In another embodiment, the capture molecules are coated on magnetic beads. Beads are introduced into the assay solutions before or after the biochemical reaction. Selected molecules are captured to the beads through molecular binding process. The pillars 32 of the capture carrier 30, which are made of magnetizable materials in this embodiment, are magnetized and capture the molecules by attracting the magnetic beads to the pillar surface.

In many assays, one or more capture carriers are used multiple times to capture different molecules from one or more assay carriers. The captured molecules form “sandwiched” or molecule layers on the surface of the pillar. For example, the pillar of the capture carrier can first be coated with a specific antibody. The antibody captures the antigen in the through-hole on the first assay carrier. After rinsing, the pillar can be inserted into another through-hole that is filled with a solution of a fluorescent labeled antibody. The new antibody is captured by the pillar to form an antibody-antigen-antibody (labeled) sandwich, which provides detectable signal at a very high specificity.

8. Washing the Capture Carrier

After molecule extraction, the capture carrier can be rinsed in bulk liquid with a certain designed stringency to remove non-target molecules that bind non-specifically on the pillar surface.

9. Releasing Molecules from the Capture Carrier

In many assays, more than one type of molecule is initially captured by the capture carrier. Therefore it may be highly desirable to release the captured molecules back to a solution for further biochemical reaction. Such a release can be achieved by inserting the pillars of the capture carrier into corresponding through-holes of an assay carrier loaded with a special releasing solution. The solution can have suitable chemicals or enzymes to separate the captured molecules from the pillar surfaces of the capture carrier. Examples of such releasing reagents include enzymes such as proteases, for example proteinase K or chymotrypsin, solutions of high ionic strength such as a concentrated NaCl solution, solutions containing a high concentration of inorganic phosphate, and others. Vigorous agitation and other washing enhancement measures can be introduced to encourage the release of the molecules into the solutions. In a specific embodiment, the inner wall of the through-hole can again be coated with capture molecules to bond the released molecules on the surface of the through-hole on the assay carrier. After releasing, the capture carrier can be removed from the assay carrier.

10. Signal Amplification of Attached Molecules

As described in previous steps, the signal producing molecules can be captured and formed on either pillar tips of a “capture & detect” carrier or the sidewall of a through-hole on an assay carrier. Signals generated from these attached molecules can be amplified for detection. The amplification can be achieved through enzymatic reaction, where bulk reagent containing suitable enzymes or substrates can be loaded into through-holes of the assay carrier or by flooding the pillars of the capture carrier to amplify the optical signal. Example enzymes for such amplification are: alkaline phosphatase, horseradish peroxidase, β-galactosidase, urease, and others.

11. Signal Detection

Excitation and detection of signals can be conducted either on a capture carrier or on an assay carrier. A wide range of detection methods is applicable to this and other embodiments of the invention. The detection of biomolecular reactions may be carried out using any type of detectable signal, such as, for example, colorimetric, fluorometric, electrochemical, radioactive and/or electronic detection labels. Optical detection modes may include absorption, colorimetric, chemical luminescence, fluorescence intensity, fluorescence correlation spectroscopy (FCS), fluorescence-resonance energy transfer (FRET), time-resolved fluorescence and fluorescence polarization. The reaction may be followed using standard detection techniques such as those involving optical, CCD, CMOS or laser optics. Furthermore, built-in detectors such as the optical waveguides described in PCT Publication WO 96/26432 and U.S. Pat. No. 5,677,196, surface plasmons, and surface charge sensors are compatible with many embodiments of the invention. Other types of detection systems and methods are described in greater detail in U.S. patent application Ser. No. 10/080,274, entitled “Method and Apparatus Based on Bundled Capillaries for High Throughput Screening” by Shiping Chen et al., filed Feb. 19, 2002, incorporated by reference herein in its entirety.

When the capture carrier (the “capture and detect” carrier in particular) is used as the platform for optical detection, the excitation and detection optics can be focused on the tip of the pillar where the signal producing molecules are attached, as shown in FIG. 18c. In this way, signals generated by molecules that bind non-specifically to the areas outside the pillar tip are out of focus and will be greatly reduced.

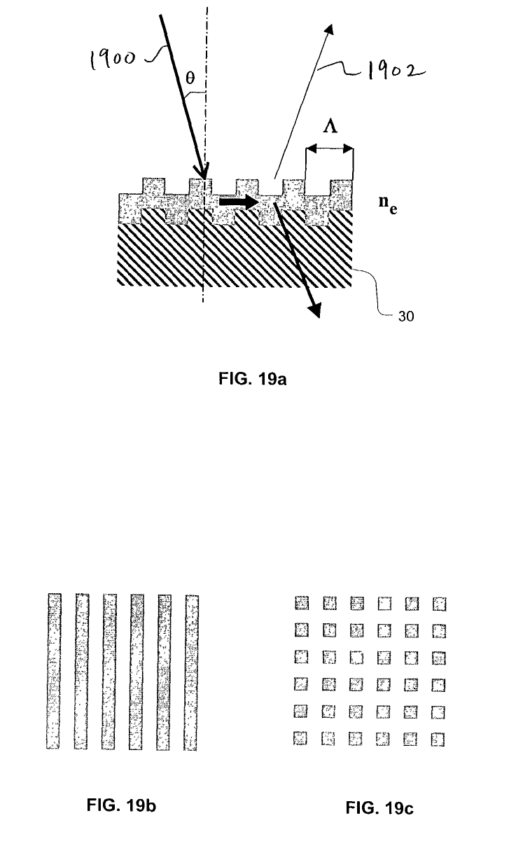

In fluorescence-based detection approach, the optical signal can be enhanced greatly by fabricating a grating layer on the surface of the pillar tip. The configuration of the grating layer is illustrated in FIG. 19a, where the refractive index and the pitch of the grating layer is n_{g} and Λ, respectively; θ is the incident angle of the excitation beam 1900. Under the condition, sin θ=n_{g}±mλ/Λ, the incident beam can be coupled into surface guided wave traveling transversally in the grating layer. The grating layer can be designed to have a thickness in the range of 10 nm˜500 nm. At this thickness, the guided wave generates a strong evanescent field that efficiently excites the fluorescent marker attached to the grating surface. By carefully selecting the grating parameters, it is possible to minimize the light coupled out of the wave guide into reflected beam. In this way, the signal to noise ratio of the detected signal 1902 can be improved greatly. The operating principle of such a grating layer has been described in U.S. Pat. No. 5,822,472, to Danielzik et al., incorporated by reference herein in its entirety.

In the Danielzik patent, however, a one-dimensional grating is used, as illustrated in FIG. 19b. Such a grating utilizes a specific orientation of the excitation beam and its polarization in order to be efficiently coupled into a guided wave. In many commercially available scanners, the excitation beam is normally perpendicular to the substrate surface and its polarization is not well defined and may change from scanner to scanner or change from manufacturer to manufacturer. FIG. 19c shows a two-dimensional grating structure, which is not sensitive to the polarization orientation. This two-dimensional grating structure can be produced with a structure similar to that described in the Danjelzik patent, but instead of a single set of gratings in a single direction, a second set of gratings is added. This second set of gratings can be identical to the first set, but oriented perpendicular to the first set to thereby overcome the sensitivity to the polarization orientation.

The grating layer described above can also be applied to any substrate to the enhance the signals generated by reagents deposited on the substrate.

II. Sample Assays

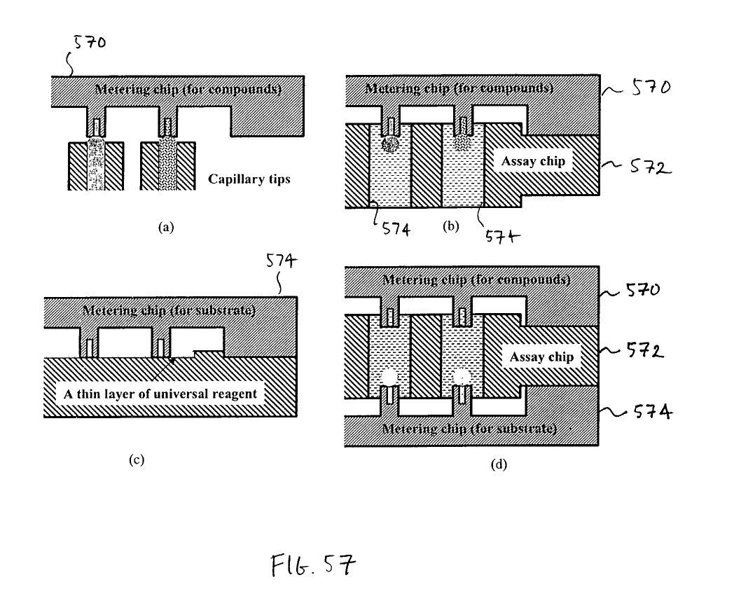

FIG. 57 illustrates one sample assay which may be performed in accordance with embodiments of the present invention. This assay can be used in various high throughput screening applications and involves a substrate and an enzyme as two URs and a chemical compound library as IRs. In FIG. 5a, the chemical compound library is loaded into the cavities of a metering carrier 570 from the capillaries 17 of loading station 14.

Once loaded, the compound can be stored and sealed in the pillar cavities of the metering carrier 570 for shipment to the end user. The end user can be located at a remote location where the HTS operation can be performed using an assay station 16. In this example, the loading station 14 and the assay station 16 are provided at separate locations. In another embodiment, the compound can be frozen after loading and shipped to the end user while in a frozen state. In yet another embodiment, the compound solution is allowed to dry after loading. Before assaying, the loaded compound can then be either thawed or a suitable solvent, such as pure DMSO, can be loaded into each pillar to redissolve the compound.

At the assay station 16, the enzyme is universally loaded into all of the through-holes of an assay carrier 572. The enzyme can be universally loaded using the various loading methods described herein. Next, the metering carrier 570 loaded with the compound library is coupled with the assay carrier 572, such that the pillars of the metering carrier 570 are aligned with and inserted into the through-holes of the assay carrier 572, as shown in FIG. 57b.

In FIG. 57c, a substrate solution is universally loaded into a second metering carrier 574 by inserting the pillars of the second metering carrier 574 into a thin layer of the substrate fluid. The substrate metering carrier 574 is then coupled with the assay carrier 572 such that the pillars of the substrate metering carrier 574 are inserted into the opposite ends of the through-holes in the assay carrier 572 from metering carrier 570, as shown in FIG. 57d. By coupling the second metering carrier 574 to the opposite side of assay carrier 572, both first metering carrier 570 and second metering carrier 574 can be retained in the coupled position simultaneously, thereby extending the time available for the reagents contained in the pillars to thoroughly diffuse through the enzyme solution in the assay carrier 572.

After all three reagents are thoroughly mixed, the two metering carriers 570 and 574 can be removed and the contents of the assay carrier 572 allowed to incubate. Alternatively, the incubation can be performed while the metering carriers 570 and 574 are still coupled to the assay carrier 572. One or both of the metering carriers 570, 574 can be decoupled and removed to provide visual access to the contents of the assay carrier 572 for signal detection. Although the metering carriers 570, 574 may retain some residual volume of fluid in the pillar cavities, the experimental result is not affected because the metering carriers 570, 574 are coupled to the assay carrier 572 for a length of time sufficient to thoroughly mix the reagents into a uniform solution.

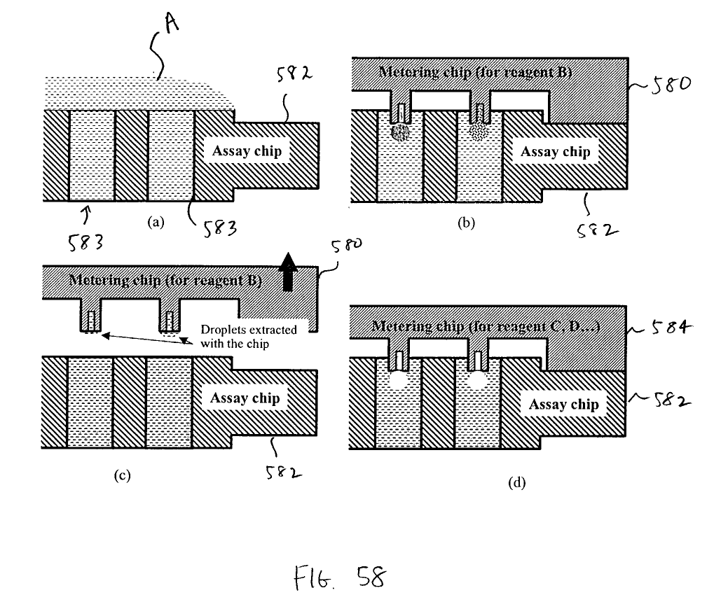

FIG. 58 illustrates another embodiment in which more than three different reagents are mixed at different times in the assay carrier 582. First, a universal reagent A is loaded into the through-holes 583 of the assay carrier 582, as shown in FIG. 58a. Next, a metering carrier 580 loaded with a reagent B (which can be either an IR or a UR) is coupled with the assay carrier 582, as shown in FIG. 58b. The mixing or reaction can be observed in real time from the side of the assay carrier 582 opposite the metering carrier 580. After reagents A and B are thoroughly mixed, the metering carrier 580 can be decoupled and removed, as shown in FIG. 58c. Although the removal of the metering carrier 580 may extract a small amount of fluid from the through-holes 583, because the size of the pillars on the metering carrier 580 are small relative to the volume of the through-holes 583 of the assay carrier 582, this removal will not substantially alter the total fluid volume in the through-holes 583. The amount of fluid extraction from the assay carrier 582 can be further reduced by making the outer surface of the pillars hydrophobic.

One or more additional reagents can be added into the through-holes 583 of assay carrier 582 using one or more additional metering carriers 584, as shown in FIG. 58d. As each metering carrier 584 is coupled with the assay carrier 582, any reaction occurring in the through-holes 583 can be detected in real time from the opposite side of the assay carrier 582.

A. Cell-Based Assays

FIGS. 59a-59b illustrate an embodiment of the present invention for performing cell-based assays. In accordance with this embodiment, cells can be cultured directly in the through-holes 593 of an assay carrier 592. Alternatively, the cells can be loaded into the through-holes 593 using the immersion or flooding methods described above. For assays involving suspension cells, a plain through-hole structure as described above can be used. For assays with adherent cells, various systems may be used. In FIG. 59b, a through-hole 593 having a cover 594 enclosing one end of the through-holes 593 is used. This cover 594, which in some embodiments is transparent or can include a transparent portion, can be fabricated by bonding a glass sheet onto a silicon wafer that forms the assay carrier 592.

Additional reagents can be added into the through-holes 593 using additional metering carriers, as described above with respect to FIGS. 58a-58d. Any reactions can be observed in real time through the transparent portion of the cover 594.

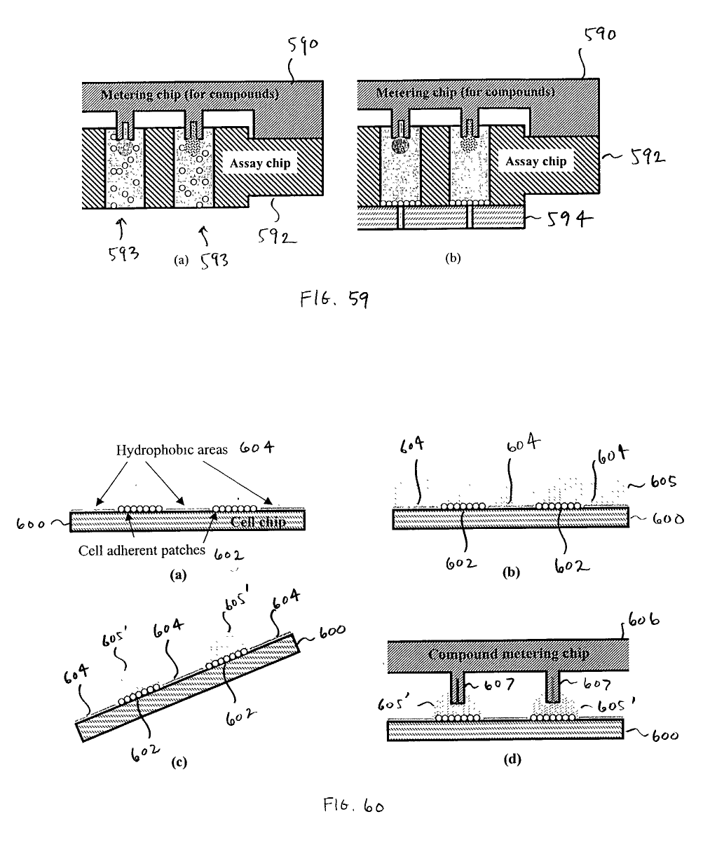

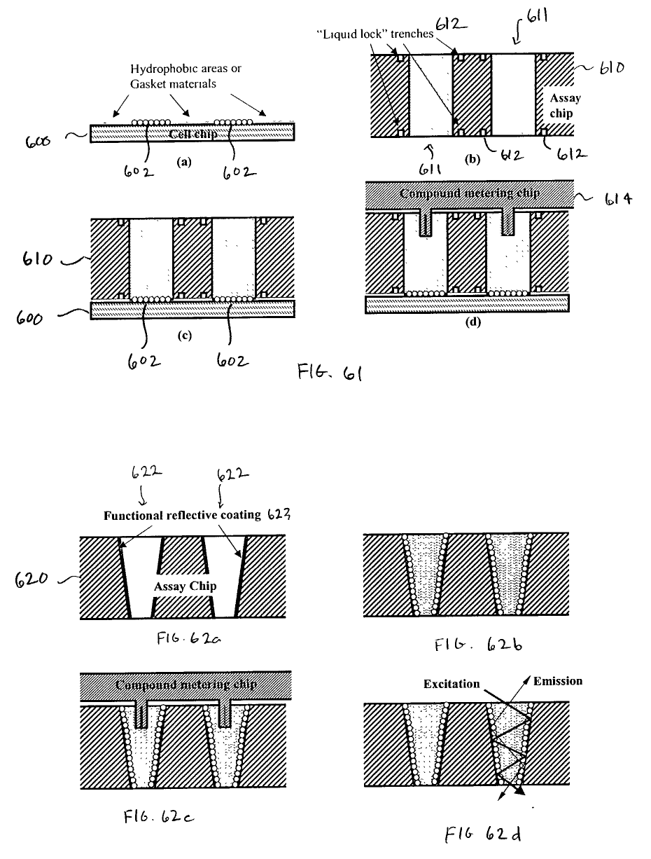

FIGS. 60a-60d illustrate an alternative approach to performing adherent cell-based assays. In this embodiment, an array of surface patches 602 are formed on a surface of a cell carrier 600. Each of these surface cell patches 602 have a hydrophilic surface coating suitable for cell growth and/or adherence. The surface areas 604 outside of the cell patches 602 are made to be hydrophobic, as shown in FIG. 60a. In some embodiments, photolithography and etching can be used to form the cell patches 602 such that they are physically elevated above the other surface areas 604.

Cells can be cultured directly on the carrier 600 or can adhere to the cell patches 602 after being cultured elsewhere. After the cells are adhered to the cell patches 602 of the carrier 600, a reagent or other medium 605 is used to flood the surface of the carrier 600, as shown in FIG. 60b. The carrier 600 can then be tilted, rotated, or otherwise agitated to remove excess fluid from the surface of the carrier 600 while retaining a droplet 605′ of fluid at each cell patch 602, due to the hydrophilic nature of the cell patches 602, as shown in FIG. 60c. Finally, a metering carrier 606 loaded with compounds is aligned with the array of cell patches 602 and each pillar 607 is inserted into a droplet 605′ to introduce the compound contained in the cavity of the pillar 607 to the droplet 605′, as shown in FIG. 60d. If the carrier 600 is formed of a transparent material or includes transparent portions aligned with the cell patches 602, the reaction can be observed from the underside of the carrier 600.

In an alternative embodiment, the cell carrier 600 can be used in assays with suspended cells instead of adherent cells. In this embodiment, the cells are suspended in a buffer which is deposited onto the surface of the carrier 600 and retained in the hydrophilic cell patch regions 602. The droplets contained in each cell patch 602 are then mated with pillars 607 on a metering carrier 606.

In yet another embodiment, the carrier 600 described above can be used in an enzymatic assay, in which the droplet on the carrier 600 is the UR, and other reagents (e.g., IR reagents) are added using a metering carrier 606.

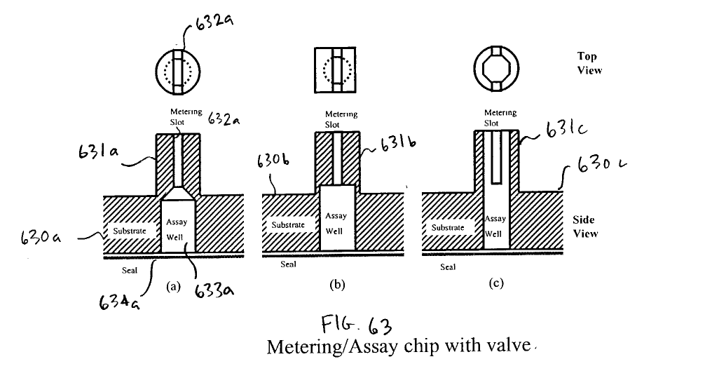

FIGS. 61a-61d illustrate another embodiment used for adherent cell assays. This illustrated embodiment enables the assay to be performed while preventing fluid cross-talk between the through-holes 611 of the assay carrier 610. In some embodiments, the surface areas of the assay carrier 610 outside of the through-holes 611 are made hydrophobic and/or coated with a layer of gasket material, such as polydimethylsiloxane (PDMS). In some embodiments, trenches 612 are formed around the opening of each of the through-holes 611 to inhibit fluid from flowing of the through-holes 611 past the trenches 612. In some embodiments, the openings of each of the through-holes 612 include a ridge portion which is elevated above the remaining surface of the carrier 610.

After the assay carrier 610 is loaded with a universal reagent or medium, as shown in FIG. 61b, the through-holes 611 are aligned with the cell patches 602 on a cell carrier 600 and the assay carrier 610 is coupled with the cell carrier 600, as shown in FIG. 61c. A metering carrier 614 loaded with a compound is then coupled with the assay carrier 610. If the cell carrier 600 is transparent or includes transparent regions, the reaction can be optically detected through the cell carrier 600.

FIG. 62 illustrates yet another embodiment for performing adherent cell assays. As shown in FIG. 62a, an assay carrier 620 includes through-holes 622 having a reflective coating 623 provided on the through-holes' inner walls. This coating can be provided by coating a metallic layer such as gold onto the inner wall of the through-holes 622. The inner walls of the through-holes 622 are also functionalized for cell adherence using any of the various methods commonly used by those of ordinary skill in the art.

In the illustrated embodiment, the through-holes 622 are formed in a conical shape, which can assist in the metal coating and light excitation during read out. The adherent cells can be either grown inside the through-holes 622 or loaded into the through-holes 622 after being grown elsewhere and adhered to the through-hole walls, as shown in FIG. 62b. Compounds or other reagents can be loaded into the through-holes 622 using any of the methods described above. Finally, in FIG. 62d, the assay carrier 620 is read using, for example, luminescent or fluorescent methods. In both fluorescent and luminescent detection modes, the reflective inner surfaces of the through-holes 622 assist in guiding the emission light towards the openings at the top and bottom of the through-holes, where the light can be collected by detection optics. In fluorescent applications, the reflective wall can enhance the excitation efficiency because the excitation beam can be reflected off of the inner walls multiple times to exit the through-hole 622, as shown in FIG. 62d.

III. Additional Loading Methods

As described above, various methods can be used for loading liquids into the liquid carriers. The following are additional loading methods which can be used in other embodiments of the present invention.

A. Liquid Delivery Using a Staging Device

In accordance with embodiments of the present invention, a “three-step” delivery process for delivering very small volume of liquids at different locations in parallel is provided. In step one, different liquids are delivered in parallel to a staging device. The volumes delivered to this staging device may be not be very uniform or precise. In step two, the volumes of liquids on the staging device are adjusted to precisely the amount desired by trimming out excess liquids or topping off unfilled volumes. In step three, the liquids on the staging device are delivered to a final destination.

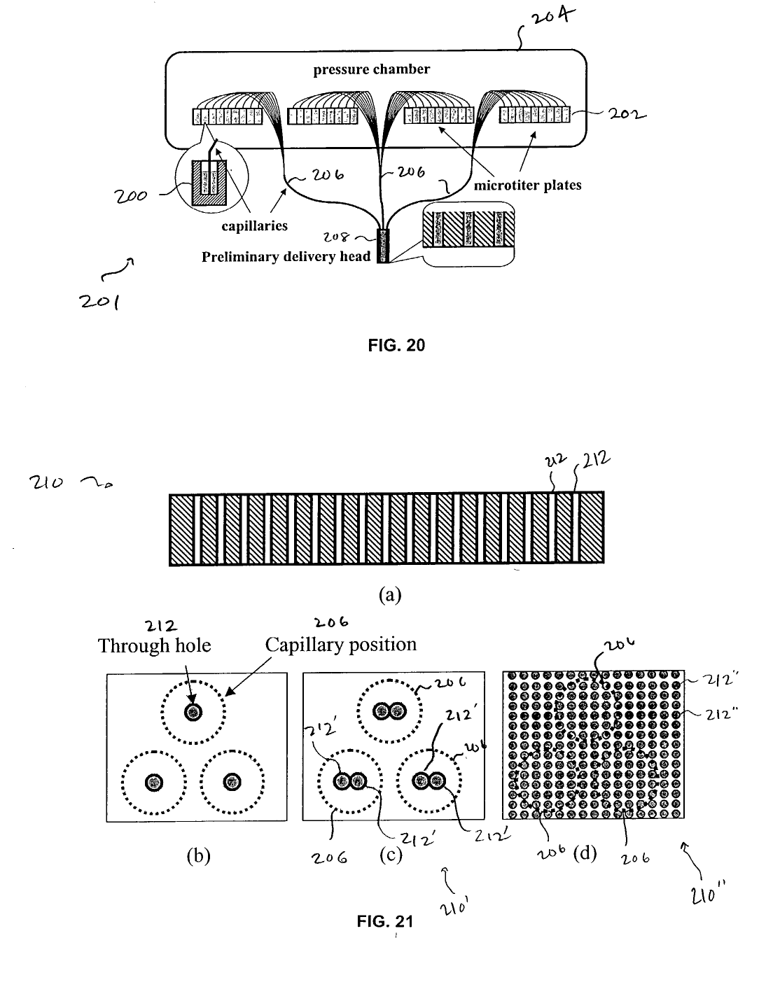

FIG. 20 shows an initial delivery sub-system 201 comprising a pressure chamber 204 and a capillary bundle 208 in accordance with embodiments of the present invention. The liquids to be delivered are stored in individual reservoirs 200, which could be wells 200 in standard microtiter plates 202. These reservoirs 200 are placed inside the pressure chamber 204. The proximal end of one or multiple capillaries 206 are inserted into each reservoir 200, which guide the liquid towards the distal end where all capillaries are bundled together. The liquids are driven from the proximal end of the capillaries 206 to the distal end by, for example, one or a combination of the following mechanisms: pressure, gravity, capillary force, electric field, or magnetic field. The bundle 208 holds the distal ends of capillaries 206 in a specific spatial pitch and pattern. Exemplary sub-systems capable of delivering a very large number of small quantities of different liquids in parallel are described in greater detail in U.S. patent Publication Ser. No. 2002/0051979, entitled “Microarray Fabrication Techniques and Apparatus”, and U.S. patent Publication Ser. No. 2001/0053334, also entitled “Microarray Fabrication Techniques and Apparatus”, each of which is incorporated by reference in its entirety herein.

FIGS. 21a-21d show various configurations of staging devices. FIG. 21a shows a cross-section of staging device 210 comprising a flat plate or substrate having a large number of through-holes 212 linking the top and bottom surfaces of the substrate. The length and diameter of the through-holes can be manufactured to a high degree of precision. The volume inside each through-hole can therefore be precisely defined. In most cases, all through-holes are parallel to each other and have the same diameter, although variations are possible. As a result, the inner volume of each hole is the same. For example, a hole having a 10 μm diameter and 1 mm length has an interior volume capable of holding about 80 pl of liquid and a cluster of one hundred 1 μm holes of equal length will hold the same amount of liquid.

In the embodiment shown in FIG. 21a, the pitch and pattern of the through-holes 212 are the same as that of the capillaries 206 in the bundle 208 in the initial delivery sub-system 201. A top view of this arrangement is shown in FIG. 21b. In other embodiments, it is also possible to have multiple holes 212′ in place of each single hole 212, as shown in FIG. 21c. In yet another embodiment, shown in FIG. 21d, the through-holes 212″ have a much denser pitch than that of the capillaries 206 in the capillary bundle 208. The top and/or bottom surfaces of the staging device 210 can be made hydrophobic and the inner surfaces of the through-holes 212 can be made hydrophilic.

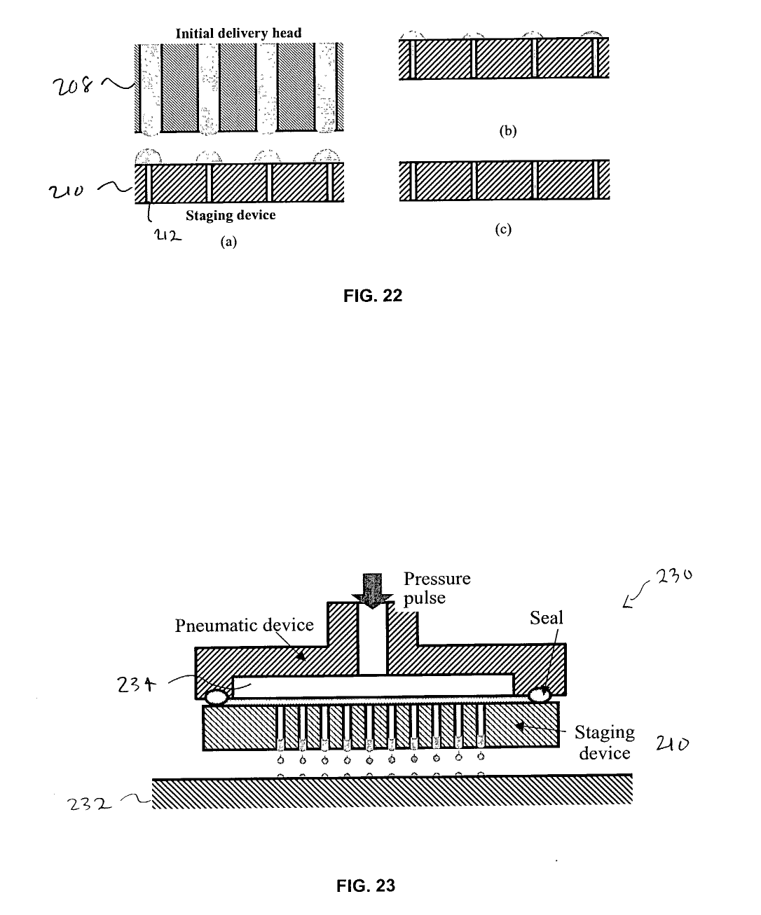

When the above described sub-system 201 and staging device 210 are used for liquid delivery, the initial delivery sub-system 201 first loads liquids into the through-holes 212 of the staging device 210. When the embodiment shown in FIGS. 21a-21b is used, the staging device 210 is aligned with the capillary bundle 208 such that each capillary 206 deposits a droplet on top of a single through-hole (or a group of through-holes 212′, 212″, as shown in FIGS. 21c-21d), as shown in FIG. 22a. The capillary force or a vacuum pressure under the staging device 210 draws the liquid from the delivery head 208 into the through-hole(s) 212, as shown in FIG. 22b. Once the liquids have filled up the holes 212, the liquids can be held inside by the capillary force. It may be desirable that the initial delivery sub-system 201 provide a greater volume of liquids than that required to fill up the hole(s) 212.

As shown in FIG. 22b, there may be extra liquid protruding from the top and/or bottom surfaces of the staging device 210. This excess liquid can be removed by several different methods, including, for example, evaporation, vacuum suction, or absorption using a suitable blotting material. After removal of the excess liquids on the top and bottom surfaces, each hole now holds a precise amount of liquid determined by the diameter and length of the holes, as shown in FIG. 22c.

The third step is to deliver the liquid contained within the through-holes 212 of the staging device 210 to a target substrate 232. In one embodiment shown in FIG. 23, the staging device 210 is fitted into a pneumatic outlet 230 to create a small, sealed chamber 234 on top of the staging device 210. The staging device 210 can be positioned and aligned above the target substrate 232 where the liquid in the holes will be delivered. Target substrate 232 could be, for example, wells in a high density microtiter plate, a microarray substrate, an assay carrier 50, or a reagent metering carrier 30. A pulsed pressure is introduced into the chamber 234, which causes all or a portion of the liquids in the through-holes 212 to be ejected out of the bottom surface and onto the target substrate 232. When the pressure is used to eject all of the liquid contained in the through-holes 212, the volume of liquids delivered will equal the amount held inside a hole 212 or a cluster of holes 212, thereby enabling the delivery of predetermined precise volumes of liquid to the target substrate 232.

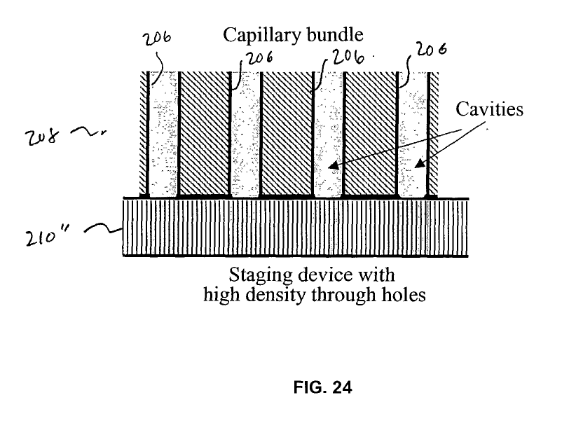

The staging device 210″ shown in FIG. 21d can be used in the same way as described above except that the loading processing may be slightly modified. As shown in FIG. 24, the facets of the capillaries 206 should be positioned to directly abut the top surface of the staging device. The liquid in each capillary 206 can be drawn into the cluster of through-holes 212″ that are directly beneath the cavity of the capillary 206 due to larger capillary force in the smaller holes 212″. As described above, a partial vacuum can also be introduced under the staging device 210″ to draw liquid into the holes 212″. The staging device 210″ can eliminate the need for precise alignment between the capillary bundle 208 in the initial delivery sub-system 201 and the through-holes 212″ in the staging device 210″ because the pitch density of the holes 212″ on the staging device 210″ is substantially higher than that of the capillary bundle 208 so that the cavity of each capillary 206 can cover a large number of through-holes 212″ on the top surface of the staging device 210″.

In other embodiments, the third step of delivering the liquids from the staging device 210 to the target substrate 232 can be omitted. Instead, the loaded staging device 210 can be used as a loaded assay carrier. In yet other embodiments, the loaded staging device can be used as a loaded reagent metering carrier to deliver liquids to an assay carrier. Various combinations and alternatives are possible and contemplated.

B. Compound Library Carrier

In accordance with embodiments of the present invention, various capillary array compound libraries, methods of making capillary array compound libraries, and methods of using capillary array compound libraries are provided. Embodiments of the invention also provide various capillary designs that may be incorporated into libraries and methods of the invention, including those described in each of the other applications and other documents mentioned herein.

The substrate of a capillary array compound library may be a chip or carrier 250 having an array of capillaries 258. In some embodiments, the carrier 250 may be used in the methods and systems described herein in place of an assay carrier 50, with the capillaries 258 serving as the through-holes 54.

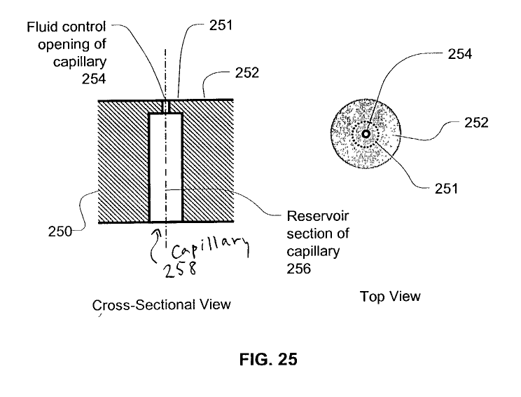

FIG. 25 shows a cross-sectional view and a top view of an exemplary capillary 258. Each capillary 258 (or through-hole) in the array includes an opening 254 for liquid control and an enlarged reservoir section 256, which can be used for both compound storage and as a reaction chamber. A region 251 in the immediate vicinity of the liquid control opening 254 on the top surface of the carrier may be coated to be hydrophilic, and/or the remaining area 252 on the top surface may be treated to be hydrophobic as illustrated. The device can be fabricated using, for example, drilling, lithography, deep reactive ion etching (DRIE), extrusion, bonding capillaries to a wafer, and other processes that are well known in the semiconductor and fiber optics industry.

To use the capillary array carrier 250 described above, compounds are first loaded into each through-hole 258 using, for example, a compound loading station such as the stations described above with respect to FIGS. 1 and 20. The compound in liquid form can be stored in the reservoir section 256 of the through-hole 258. The loaded capillary array can be sealed by attaching a film on the top and bottom surfaces of the carrier 250. This can prevent evaporation during shipping and storage.

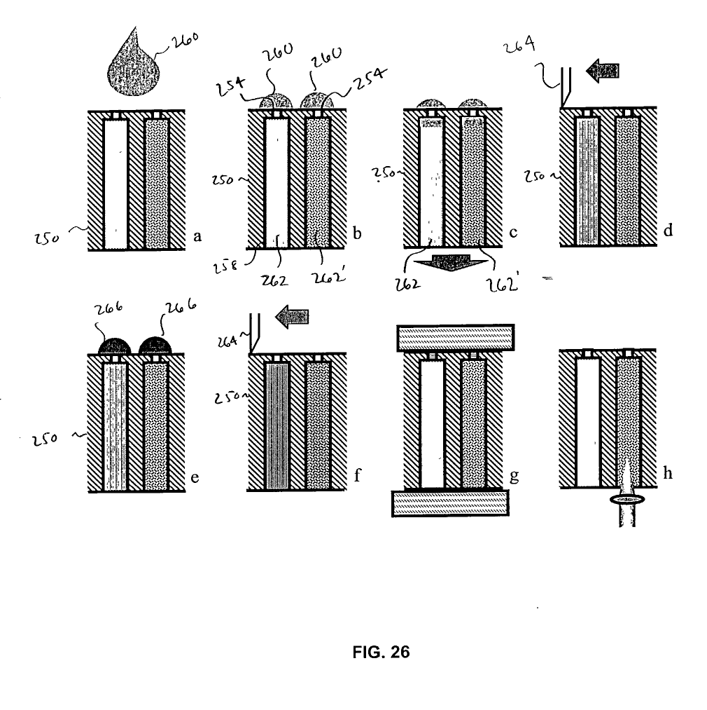

The loaded compound library carrier 250 can be processed in an assay station 16 to carry out various assays, such as, for example, the enzymatic screening assay illustrated in FIGS. 26a-26h. In FIG. 26a, the assay substrate liquid 260 is applied onto the top of the carrier. The substrate liquid 260 will be held in the hydrophilic regions 251 surrounding the small capillary openings 254, as shown in FIG. 26b. A negative pressure can be applied to the bottom of the carrier 250, which can draw a certain quantity of the substrate liquid 260 into the reservoir section 256 of the through-hole/capillary 258. The substrate liquid 260 mixes with the particular compound 262 and 262′ in each reservoir section 256, as shown in FIG. 26c. In FIG. 26d, excess substrate liquid 260 remaining on the top surface of the carrier 250 can be removed by, for example, drawing a blade 264 across the top surface of the carrier 250. In FIG. 26e, an enzyme 266 is applied on to the top surface of the carrier 250 and is held in the hydrophilic regions 251 because of hydrophilic attraction. In FIG. 26f, a negative pressure applied to the bottom of the carrier 250 draws an amount of enzyme 266 into the reservoir 256, which mixes with the compound 262/substrate 260 mixture with the enzyme 266. Excess enzyme 266 can be removed from the top surface of the carrier 250 as described above with respect to FIG. 26d. The through-holes 258 can be resealed by capping the top and bottom surfaces of the carrier 250 to prevent evaporation during incubation if desired, as shown in FIG. 26g. The resulting reaction and/or association can be detected optically in a number of ways. One way is to apply a positive pressure from the bottom of the carrier 250 to push some or all of the contents (compound 262, substrate liquid 260, and enzyme 266) of the through-holes 258 onto the top surface of the carrier 250 to detect the presence or absence of reaction and/or association from the top of the carrier 250. Alternatively, the compound/substrate/enzyme mixture can remain in the reservoir. As shown in FIG. 26h, the liquid in the through-hole 258 is excited and read from the large opening at the bottom or the small opening at the top of the through-hole 258. In some embodiments, at least the top portion of the carrier 250 adjacent to the small opening 254 of the capillary 258 can be transparent or translucent. Excitation and read out can be carried out from the top of the carrier 250. The above-described format can enable the mixing and reaction to take place inside the through-hole 258 of the carrier 250, thereby minimizing the evaporation during incubation.

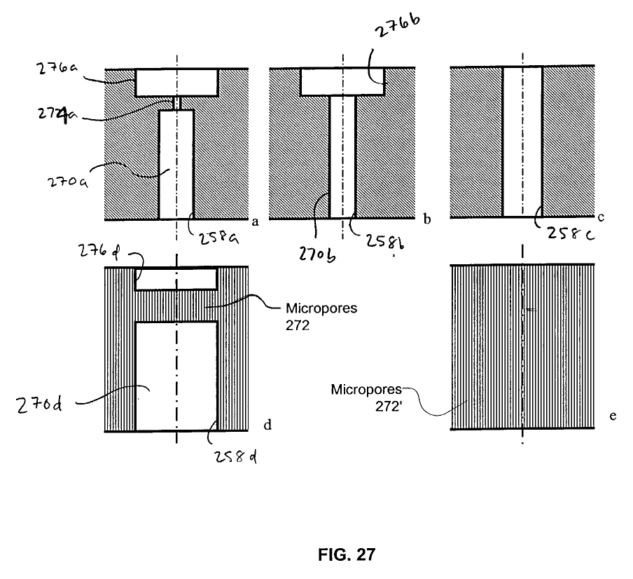

FIGS. 27a-27e show some alternative designs of capillaries 258 incorporated into compound library carriers 250, which can all be used in the same ways described above. FIG. 27a illustrates a capillary 258a having a reservoir section 270a, a liquid control opening 274a, and a well 276a at the top portion of the capillary 258a. A well and carrier may be configured and formed as described in U.S. patent Publication Ser. No. 2001/0055801, entitled “Liquid Arrays,” filed Feb. 22, 2001, incorporated by reference herein in its entirety.

FIG. 27b illustrates a capillary 258b having a reservoir section 270b and a well section 276b, without a narrowed liquid control opening 274a. In FIGS. 27a-27b, the well 276a-276b may serve as a reaction well or a temporary liquid holding place.

FIG. 27c depicts a capillary 258c having a constant diameter throughout. The liquid control opening 254 shown in FIG. 25 is eliminated, but the carrier can be used in the same way as the 250 in FIG. 25. FIGS. 27d-27e show different structures of the library carrier built on a substrate having a plurality of small pores 272, passing therethrough. These pores 272 can be fabricated using, for example, an electro-chemical etching process. The pores can be, for example, several nanometers to several micrometers in diameter. The size and density, thus the “open area ratio” of a porous carrier such as the one depicted in FIGS. 27d-27e can be controlled precisely in the fabrication process using conventional methods known to one of ordinary skill. Multiple pores 272 can collectively function as a liquid control device (as shown in FIG. 27d, in which the pores 272 join the well 276d above the pores 272 and the reservoir 270d below the pores 272), or a combination of flow control and liquid storage device (as shown in FIG. 27e, in which the carrier contains only pores 272′ passing completely through the carrier). For the carrier illustrated in FIG. 27e, because the pores are very small, different liquids may not mix inside the pores. The mixing and reaction may be conducted in a hydrophilic “virtual well” on the top surface of the porous carrier, for example.

C. Reagent Metering

With existing technologies, reagents are typically dispensed after they are metered using a dedicated metering device. Therefore, the dispensing process can introduce errors in the assays if the entire metered volume is not precisely and accurately dispensed. In accordance with other aspects of the present invention, systems and methods for metering reagents after dispensing are provided.

In existing systems, the liquid container, into which the reagents are dispensed, is actually a platform that performs the following functions: 1) reagent storage, 2) reagent mixing, 3) incubation for reaction, 4) result read-out. In accordance with aspects of the present invention, a destination container that performs the additional function of reagent metering is provided.

There are a variety of methods for metering reagent volume on the destination container. One method is to dispense an approximate amount of the desired volume, then precisely measure the actual volume using visual or other means, and eventually factor the actual volume into the final result mathematically. An alternative method is to dispense an excessive amount of reagent, use structural or other liquidic constraints in the container to hold a desired volume and finally to remove excess liquid from the destination container.

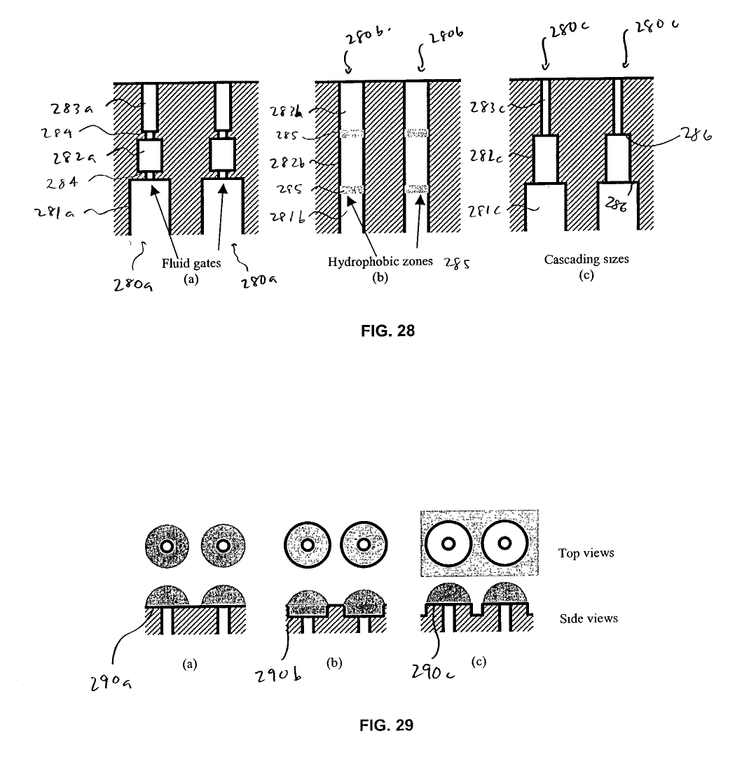

FIGS. 28a-28c show embodiments of the latter method, which may be particularly effective in applications that demand miniaturized liquid handling. As shown in FIGS. 28a-28c, the destination container comprises a through-hole 280 having a series of interconnected chambers 281, 282, 283. The inner surface areas of the through-holes 280 can be made hydrophilic. These chambers 281-282 are interconnected but separated by a liquid barrier, which impedes the flow of liquid between chambers when the pressure differential is less than a certain “bursting pressure”. This “bursting pressure” is the pressure at which the fluid begins to emerge from the chamber. The “bursting pressure” is inversely proportional to the diameter of the hole.

The hydrophobic zones can be formed by taking a wafer having through-holes passing therethrough and depositing a carbon or carbon-like layer onto the rim of the through-holes. Multiple wafers prepared in this way can then be bonded together to form a single carrier having through-holes with hydrophobic zones.

In the through-holes 280a shown in FIG. 28a, the barrier is a short narrow opening or liquid gate 284. In the through-holes 280b shown in FIG. 28b, the barrier is a short hydrophobic zone 285. In the through-holes 280c shown in FIG. 28c, the barrier is an interface 286 from a smaller to a larger chamber. In the illustrated embodiments, for clarity only two through-holes 280 are shown for each carrier. It will be understood that multiple through-holes 280 can be fabricated in parallel on a single carrier.

If the reagent is a universal reagent to be used to fill all or a plurality of through-holes 280 on a single carrier, the reagent can be applied in bulk in a flooding fashion, wherein the reagent is applied over the top surface of the carrier to be drawn into the through-holes through their top openings. While the through-holes may be described herein as being loaded from the top surface, it will be understood that either the top or the bottom surfaces can be used in various embodiments.

If each through-hole on a carrier is to be filled with a different reagent (an “individual reagent”), a delivery device such as capillary bundle or ink jet nozzle array can be used to deliver the unique liquid to each individual through-hole in either parallel or serial fashion. In such a case, liquidic features can be fabricated on the top surface of the carrier to assist in isolating different liquids contained in adjacent through-holes. Such liquidic features may be, for example, a hydrophilic patch 290a around the exit opening of the through-hole (shown in FIG. 29a), a well 290b (shown in FIG. 29b), or a raised “island” portion 290c (shown in FIG. 29c).

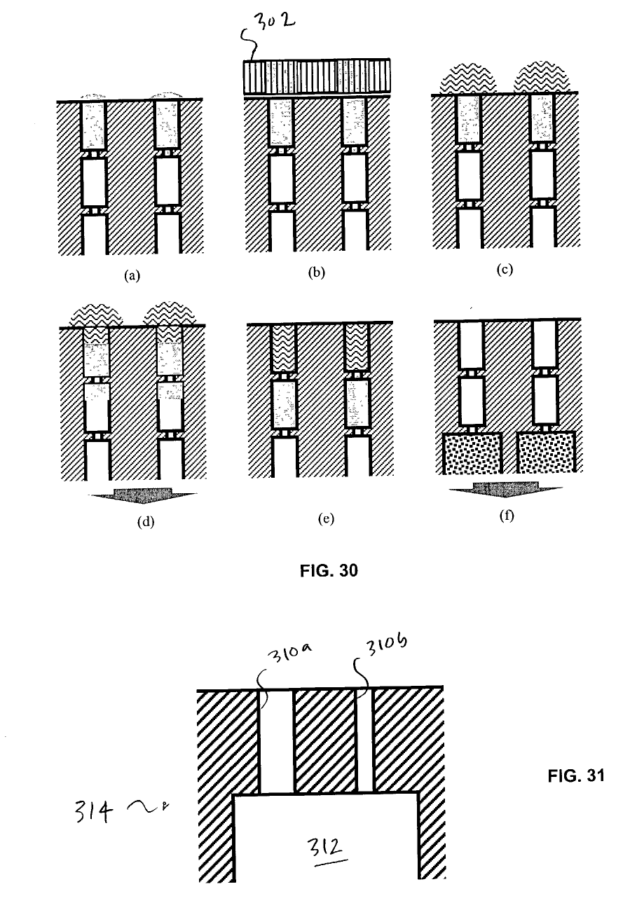

The first reagent applied to the carrier can be drawn into the first chamber 283 of the through-hole 280 and held there by capillary force. The liquid will not pass into the second chamber 282 of the hole 280 as long as the pressure does not exceed the “bursting pressure” created at the interconnecting region 284 between chambers 281, 282, 283, as shown in FIG. 30a.

After the first chamber 283 is filled, the excess liquid can be removed from the top surface of the carrier by, for example, one or a combination of the following means: 1) blotting, 2) sucking with a vacuum pressure that is smaller than the “bursting pressure” of the liquid gate 284, 3) coupling with another empty capillary array to draw up the excess liquid using capillary force; 4) wiping, and 5) blowing.

Method 3 is illustrated in FIG. 30b, where the pore size of the empty capillary array 302 that is used to remove the excess liquid should in general be larger than that in the first chamber in order to avoid the withdrawing of liquid from the chamber 283. Alternatively, the pore size of the capillary array can be smaller than the diameter of the chamber. In this case, the pores may be treated such that the pores are less hydrophilic than the chamber. In this way, liquid inside the through-hole 280 will not be removed.

After removal of the excess first reagent, a second reagent can be applied to the top surface, as shown in FIG. 30c. Then, a short duration of driving pressure is applied, which can either be a negative pressure applied to the bottom side of the carrier or a positive pressure applied to the top side. In either case, the driving pressure should be greater than the “bursting pressure” of the liquidic barrier between the first chamber 283 and the second chamber 282. This pressure will force the liquid in the first chamber 283 through the liquidic barrier into the second chamber 282. Once the barrier is burst, capillary force will take over and draw the remainder of the liquid into the second chamber 282. Because the first chamber 283 and second chamber 282 are connected, the second reagent that was deposited on the top surface will also be drawn into the first chamber 283 of the through-hole 280, as shown in FIG. 30d. Next, the excess liquid of the second reagent on the top surface can be removed and the carrier is ready for the loading of subsequent reagents, as shown in FIG. 30e. This process can be repeated as many times as the number of chambers provided in the through-hole.

After all of the desired reagents are loaded into the through-hole 280, mixing can be achieved by diffusion or alternatively, all reagents in different chambers can be pumped into a larger chamber at the end of the through-hole 280, where they can mix and incubate at a higher efficiency, as shown in FIG. 30f. The reaction results can be read from the through-hole by optical or other means.

The structure and loading method described above is sequential for each carrier. FIG. 31 illustrates an alternative carrier through-hole structure, comprising multiple chambers 310a, 310b linked to a large mixing chamber 312 in parallel. Only two parallel chambers 310a, 310b are shown for clarity, but is will be understood that any number of parallel chambers 310 connected to a single mixing chamber 312 may be used. The different reagents can be loaded in a parallel to different chambers 310 of a carrier 314.

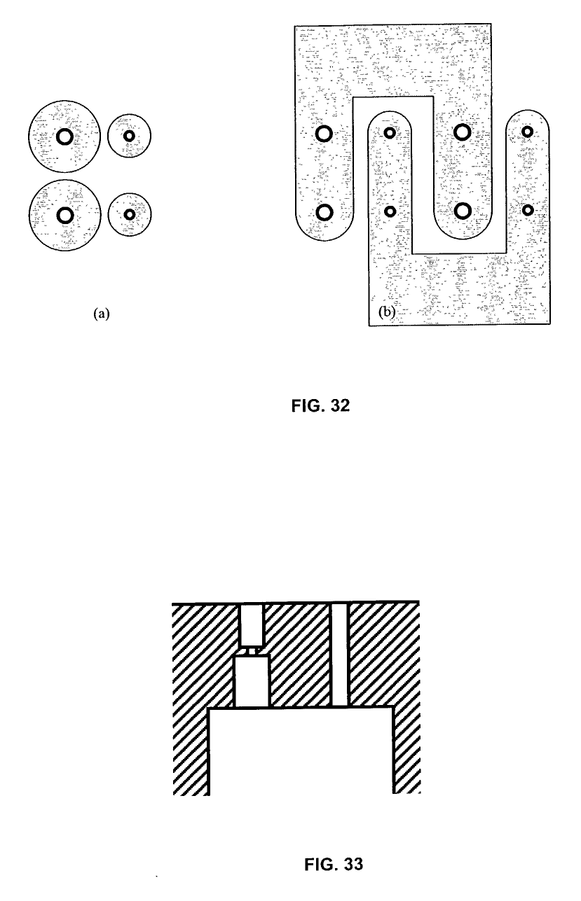

Various liquidic barrier features can be designed on the top surface of a carrier to separate the different reagents being loaded into the through-holes. FIGS. 32a-32b show some exemplary embodiments in which the shaded areas are hydrophilic and the remaining areas are hydrophobic. Fluid deposited anywhere in the shaded areas tends to remain in that area and will be absorbed by the through-hole surrounded by the hydrophilic region. Such hydrophilic patches can control the position and flow of fluid droplets on the surface of the carrier, thereby preventing the various fluids from mixing. It is also possible to combine the serial and parallel systems described above with respect to FIGS. 28-31 to arrive in a hybrid system such as the carrier shown in FIG. 33. The total number of liquid loading chambers in a carrier in many applications is not very large because many reagents can be pre-mixed in bulk prior to the delivery to the carrier.

D. Serial Dilution

In drug discovery, the production of concentration gradients of compounds can consume a considerable amount of resources. This production may involve multiple steps of dilution for each compound. As the number of compounds increases while the volumes of assays decrease, the dilution of compounds to a series of concentrations has become a bottleneck for high throughput screening on drug discovery. In accordance with embodiments of the present invention, the generation of gradients of concentration, electric charge to friction ratio, or molecular weight in a highly parallel fashion is provided. This can enable the characterization of thousands, or even millions of species simultaneously.

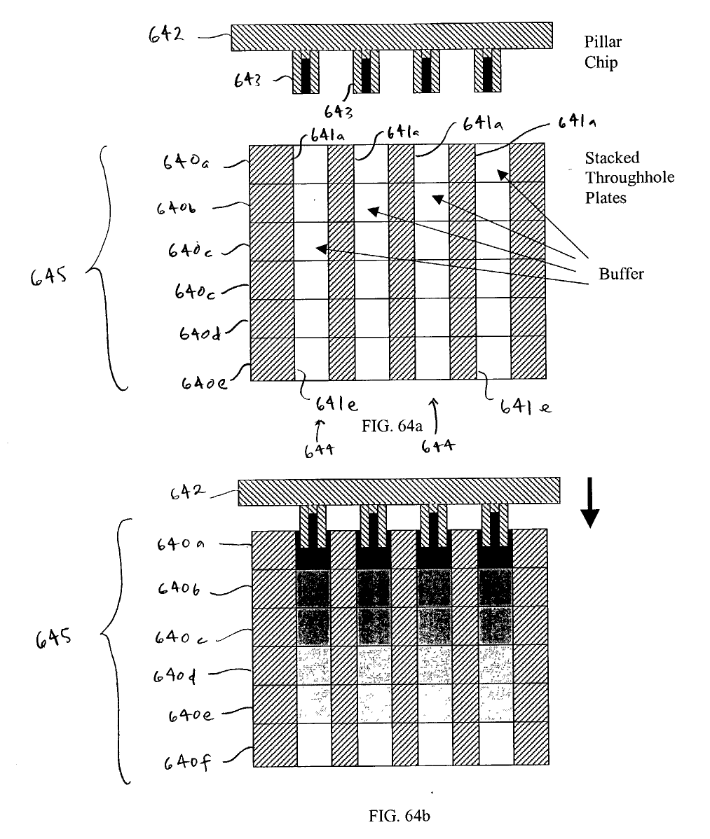

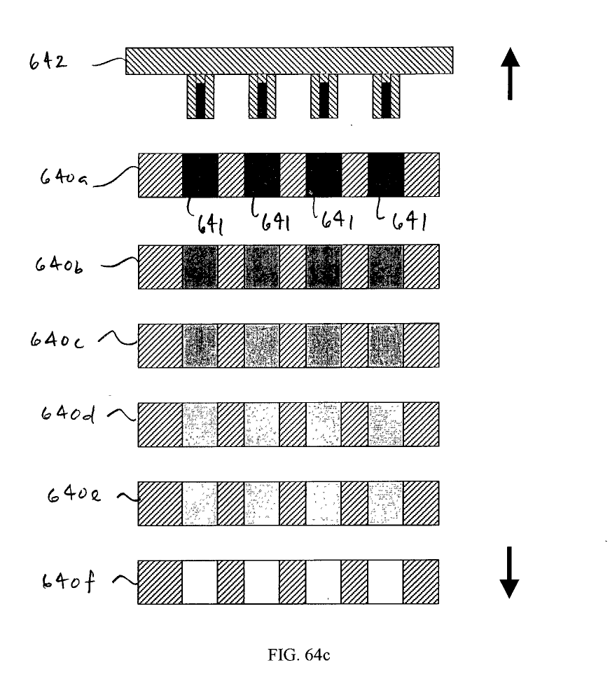

As shown in FIG. 64a, a plurality of through-hole carriers 640, each carrier 640 having an array of through-holes 641 with hydrophilic tubes and hydrophobic ends, similar to assay carrier 50 shown in FIG. 5, are provided. A metering carrier 642 having an array of hollow pillars 643 with a hydrophilic coating inside and hydrophobic coating outside, similar to reagent carrier 30 shown in FIG. 5, is also provided. The through-holes 641 on the through-hole carriers 640 and the pillars 643 on the metering carrier 642 are arranged such that when the through-hole carriers 640 and the metering carrier 642 are coupled, each of the pillars 643 is inserted into a corresponding through-hole 641 on the through-hole carrier 640.

As shown in FIG. 64a, a plurality of through-hole carriers 640 can be stacked together such that each through-hole 641 in a carrier 640 aligns with a corresponding through-hole 641 on an adjacent carrier 640. The stacked carriers form an array of extended through-holes 644. When through-holes 641 are loaded with liquid, the liquid is continuously connected in each extended through-hole 644, as shown in FIG. 64b. When the stacked carriers 640 are separated, as in FIG. 64c, the liquid that remains in each carrier 640 is a portion of the liquid previously contained in the extended through-holes 644.

In a capillary having an inner diameter of, for example, 1 μm to 500 μm, the effect of turbulence mixing and convection mixing is not significant. Mixing is predominantly by diffusion. The diffusion behavior in the tube can be described by using a one-dimensional model. More precise models can be simulated using computational fluid software, such as that produced by the CFD Research Corporation.

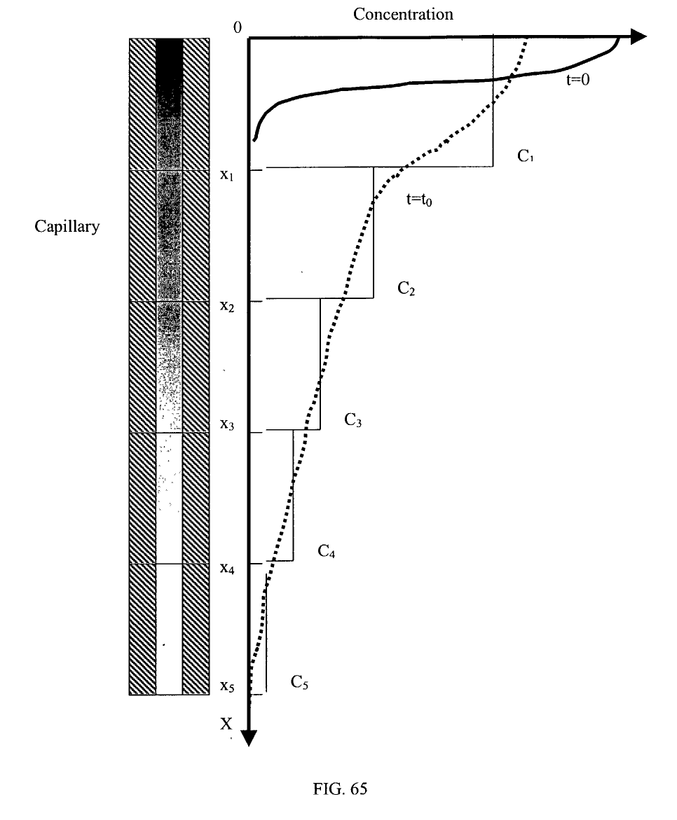

If we have a system as illustrated in FIG. 65, the diffusion equation can be written as following:

cC/ct=D d.dC [1]

C=C(t, x)

t=time

x=displacement

D=Diffusion coefficient

Do=kT/f [2]

(Einstein-Sutherland Equation)

f=6πνrₕF [3]

f=friction

ν=viscosity coefficient

rₕ=hydrated radius of particle

F=Perrin shape factor

If at t=0, the liquid column has contact with another liquid column which has a concentration c₀ at x=0, the solution of the equation is:

C(x,t)=½c₀[1+2/π^{1/2}.f₀^{x/sqroot(4Dt)}e^{-y y}dy] [4]

At t=t₀, average concentration of each segment is illustrated in FIG. 65. The average concentration can also be described as the following:

Cᵢ=1/(x₁−xᵢ₋₁).f^{xi}ₓᵢ₋₁C(x, t₀)dx [5]

1. Gradient Generated by Diffusion

In accordance with embodiments of the present invention, a concentration gradient of many species can be generated simultaneously. Each species can be, for example, a compound used in high throughput screening, a biomolecule (such as DNA, RNA or protein), or a fragment of a cell.

Different compounds can be loaded onto a pillar carrier as described above with respect to FIGS. 3-5. In addition, individual species can be directly loaded into the through-holes of a through-hole carrier using, for example, the loading station described above with respect to FIG. 1.

The loading of a buffer into the stacked through-hole carriers can be performed as described above with respect to FIGS. 9-10, or as described in U.S. patent Publication Ser. No. 2002/0001546 A1, “Methods for Screening Substances in a Microwell Array” to Hunter et al. In addition to loading buffers into the through-holes, it is also possible to use the same loading methods to load, for example, gel matrices and magnetic bead suspended solutions.

At time t=0, the loaded pillar carrier 642 is coupled with the top carrier 640a of a stacked through-hole carrier assembly 645. The pillar carrier 642 and/or the through-hole carrier assembly 645 can be agitated to enhance mixing. At time t=t0, all carriers 640a-640f are separated, as shown in FIG. 64c. The compound is allowed to diffuse within each carrier 640. The concentration of each segment of the extended through-holes from different carriers will be different (as in equation [5]).

The diffusions can be controlled by changing the viscosity of the media, or by changing the temperature of the solutions. Within the same through-hole carrier, the concentration of each species is weighted by the diffusion coefficient, resulting in a different concentration for each species. The actual concentrations of the species can be determined once the diffusion coefficients are known or determined by other methods.

2. Gradient Generated by Magnetic Force

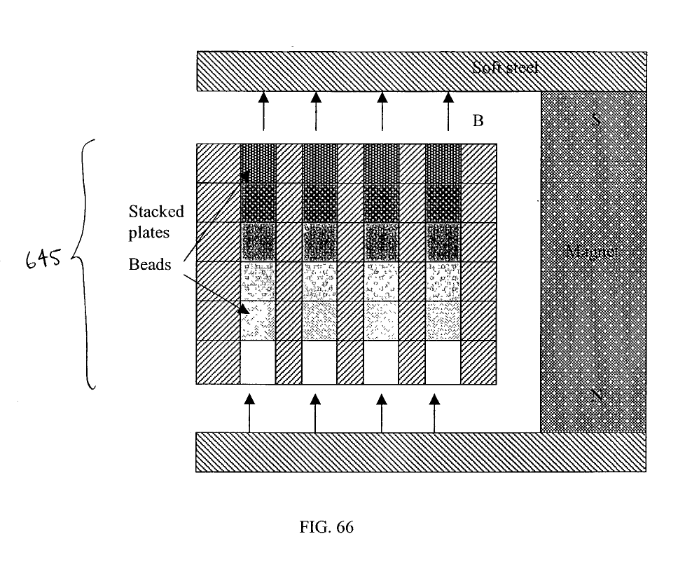

Alternatively, the stacked through-hole carrier assembly 645 can be loaded with magnetic beads suspended in buffer. The surfaces of the beads may be modified so that biomolecules can attach to the beads. When the stacked through-hole carrier assembly 645 is subjected to a magnetic field B with the through-holes 641 parallel with the direction of the magnetic field (as shown in FIG. 66), the beads are subjected to the magnetic force and travel to one end of the extended through-hole 644.

The carriers 640 can be separated after the movements of the beads reach equilibrium. In different segments of the liquid column, the beads are separated by the competition of gravity, magnetic force, and diffusion.

Alternatively, the carriers can be separated before the beads reach an equilibrium state. In different segments of the liquid column, the beads are separated by the ratio of friction over magnetic forces.

3. Gradient Generated by Electrophoresis

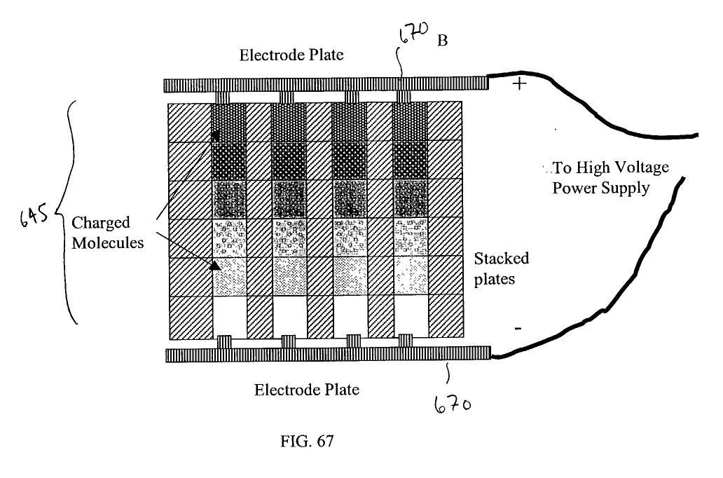

In other embodiments, electrophoresis is used to generate gradients in different segments of the stacked through-hole carrier assembly 645, as shown in FIG. 67. In this embodiment, the through-hole carriers 640 are fabricated from an isolator or coated with isolating materials. The through-holes 641 are filled with, for example, a gel matrix or buffers. A pair of electrode plates 670 are mounted to the top and bottom of the stacked through-hole carrier assembly 645. When a voltage is applied to the two electrode plates 670, molecules on one end of the through-hole carrier assembly 645 are separated by the ratio of electric charge over friction, which is related to the size of a molecule. After applying the electric field to the through-hole carrier assembly 645 for a period of time, molecules of different charge/friction ratio move to different layers of the carrier assembly 645. When the stacked carriers 640 are separated, each segment of the through-hole holds a different charge/friction ratio of molecules.

4. Application

The above-described embodiments can be used to simultaneously generate a compound library gradient for use in, for example, high-throughput screening. Assuming one has a library of 50,000 compounds. Each compound needs 15 dilutions for IC50 measurement. If one needs 3 steps for each dilution (two metering, one mixing) the total number of steps will be 2.25 million. Even if one uses a 96-multiplexed dispenser, the number of steps is still 23,437.

Using the above-described systems, the same task can be performed using far fewer steps. For example, the 50,000 compounds can be preloaded into a compound carrier on the loader (1 step), packaged (1 step), and shipped to the user (1 step). The user then stacks 15 buffer loaded through-hole carriers together (30 steps), each through-hole carrier having 50,000 holes. The compound carrier is coupled with the stacked through-hole carrier assembly for a period of time, such as, for example, a few seconds (the waiting time may depend on the diffusion coefficients of compounds) (1 step). After the period of time has elapsed, the carriers are separated (16 steps). In this way, a 15 different dilution concentrations of the 50,000 compounds can be accomplished in just 50 steps. The through-hole carriers are then ready for mixing with, for example, substrates or kinases.

As the number of through-holes in each through-hole carrier is incraesed, the efficiency of the approach can be even higher.

IV. Additional Processes

A. Combinatorial Chemical Synthesis

In accordance with embodiments of the present invention, systems for conducting highly parallel and miniaturized chemical synthesis using combinatorial chemistry principles are described.

1. Synthesis Steps

In accordance with embodiments of the present invention, a final molecule made by combinatorial chemical synthesis comprises three molecular groups: A, B and C. Each group can be one of the N possible chemicals: A₁˜Aₙ, B₁˜Bₙ and C₁˜Cₙ. It is therefore possible to make up to N³ different molecules, A₁BⱼCₖ, using combinatorial chemistry methods, where i, j, k are integers in the range between 1 and N. Unlike existing bead-based combinatorial synthesis methods, this invention can use liquid phase chemical reactions which can have superior kinetics in the synthesis reactions.

The synthesis can be performed on the above-described assay platform utilizing various combinations of the following steps: 1) load a first set of reagents (A) onto a metering carrier; 2) load a second set of reagents (B) onto a second metering carrier; 3) mix the first (A) and second (B) sets of reagents on an assay carriers; 4) load a third set of reagents (C) on to multiple metering carriers; and 5) mix the third set of reagents (C) with the previously-mixed first and second reagents (AB) on the assay carrier. The steps are described in greater detail below.

a. Load a First Set of Reagents (A) onto a Metering Carrier

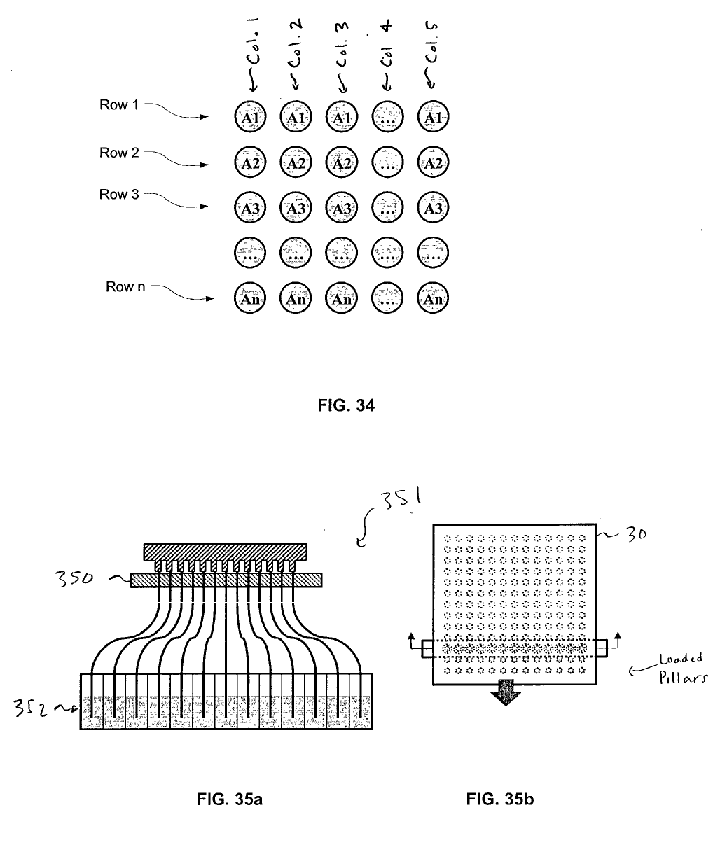

As described above, reagent group A comprises N reagents, A₁˜Aₙ, which can be loaded onto a metering carrier comprising N×N pillars. Each row of pillars is loaded with the same reagent, as illustrated in FIG. 34. It will be understood that the distinction between rows and columns on the carrier array is an arbitrary one. For the purposes of this discussion, the rows are shown as running left-to-right and the columns are shown as running top-to-bottom.

The loading can be conducted using the loading station 14, described above with respect to FIG. 1. Alternatively, a universal reagent (UR) loading device can be modified to load multiple different reagents.

FIGS. 35a-35b show side and top views of one embodiment of a multiple reagent (MR) loader 351, in which a bundle of capillaries terminates to form an integrated line array 350 on one end (proximal end) and are loose on the other end (distal end). The capillaries at the loose end are inserted into different containers 352 holding multiple different reagents, respectively. Driven by pressure or gravity, the reagents flow from the containers 352 towards the array end 350 and form small meniscuses at the tip of each capillary. At the proximal end, the capillaries are arranged to have the same spatial pitch as the pillar rows in the metering carrier 30. During loading, the metering carrier 30 is brought close to the capillary array 350 with each row of the pillars aligned to a capillary, as illustrated in FIG. 35a. By introducing a relative motion between the capillary array 350 and the metering carrier 30 along the direction of the pillar row, the reagent in the capillary can be loaded on to the slots of an entire row of pillars on the metering carrier 30. The operation of the MR loader 351 is similar to the operation of the loading bar 124 described above with respect to FIGS. 12c-12d, except that in the loading bar 12, all of the pillars are loaded with the same reagent. Using the MR loader 351, each pillar in a single row is loaded with the same reagent as the other pillars on that row, but each row is loaded with a different reagent. Therefore, each column of pillars is loaded with an identical set of reagents A₁˜Aₙ.

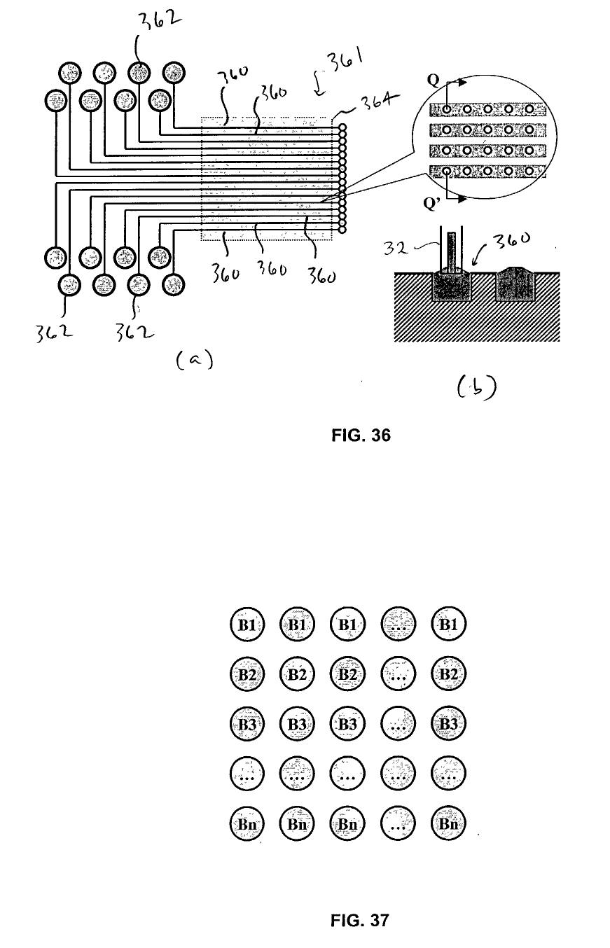

FIGS. 36a-36b show another embodiment of an MR loader utilizing a special MR loading carrier 361. As illustrated in FIG. 36a, the MR loading carrier 361 comprises an array of reagent reservoirs 362, wherein each reservoir 362 is linked to an “open top” channel 360. The channels 360 are brought to be parallel to each other in a “loading area” 364. The spacing between channels 362 equals that of the pillar rows in the metering carrier 30. The reservoirs 362 may be spaced in the same pitch as that in 96, 384 or 1536 well microtiter plates.

During loading, the reagents are delivered to the reservoirs 362 and then fill the channels 360 that link to them through capillary action. The metering carrier 30 is then brought close to the MR loading carrier 361 with its pillars 32 facing the channels 360. The pillar rows are aligned to the centers of the channels 360 and the pillars dip into the meniscuses of the reagent contained within the channel 360 to allow the slot/reservoir 34 in the pillar 32 to draw up corresponding reagents (shown in FIG. 36b).

b. Load a Second Set of Reagents (B) onto a Second Metering Carrier

Reagent group B also comprises N reagents, B₁˜Bₙ and they are loaded on to another metering carrier using, for example, the same methods as described above with respect to the first reagent group (A). The distribution of a metering carrier loaded with reagent group B is shown in FIG. 37.

c. Mix the First and Second Sets of Reagents

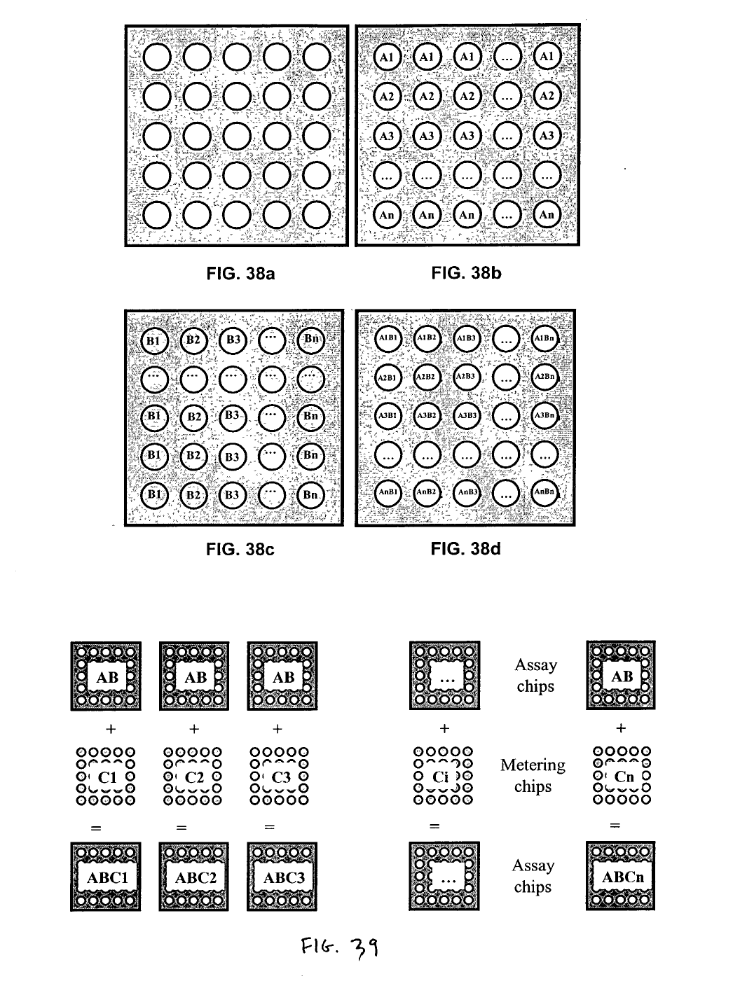

An assay carrier with through-holes can be loaded with a buffer solution using the methods and systems described above. The buffer is mixed with first reagent group A by inserting the pillars on the metering carrier into through-holes of the assay carrier, as illustrated in FIG. 38b. The metering carrier loaded with the second reagent group (B) is rotated 90° so that the pillars in each column contain the same reagent as the other pillars in that column, as shown in FIG. 38c. Finally, the metering carrier loaded with second reagent group (B) is inserted into the assay carrier already containing the buffer solution mixed with the first reagent group (A). Now the assay carrier contains all possible combinations between reagent groups A and B, as shown in FIG. 38d.

By repeating steps one to three N number of times, a total of N assay carriers containing the entire library of all possible AB combinations can be produced.

d. Load a Third Set of Reagents

Each element of a third reagent group (C) can be loaded to a metering carrier, on a one element per carrier basis, using the UR loading method described above. A total of N loaded metering carriers are produced, each loaded with C₁, C₂, . . . and C_{N}, respectively.

e. Mix the Third Set of Reagents with the First and Second Reagents

The N metering carriers loaded with reagents C₁ through C_{N} are coupled with the N assay carriers loaded with the AB combinations, shown in FIG. 38d. N assay carriers containing all possible combinations of A, B and C are then produced, as illustrated in FIG. 38e. The products, AᵢBⱼCₖ, are contained within different through-holes of the N assay carriers, which can be transferred to N² corresponding pillars on N capture carriers using the method described above.

The final two steps can be extended if there are additional reagent groups, such as D and E, to be mixed with the products of ABC combination to form more complex molecules.

In other embodiments, the combinatorial chemical synthesis can comprise two molecular groups or more than three molecular groups. Numerous embodiments are possible in which multiple carriers are used to enable combinatorial chemical synthesis of multiple groups.

2. Modified Through-Hole Having a Capture Chamber

In accordance with other embodiments of the present invention, an assay carrier includes a through-hole having a capture chamber for capturing components such as, for example, polymer beads. Systems and methods incorporating such an assay carrier can be used, for example, to integrate combinatorial chemistry synthesis with screening assay functionality in a single system to produce a flexible drug discovery platform.

A combinatorial chemistry synthesis system may comprise a synthesis carrier set and a synthesis assay station. The synthesis carrier set may comprise a metering carrier and an assay carrier. The configuration of the metering carrier may be similar to that of the metering carrier described above with respect to FIG. 1. The design of the assay carrier for synthesis may be different than the design used for screening, as described below.

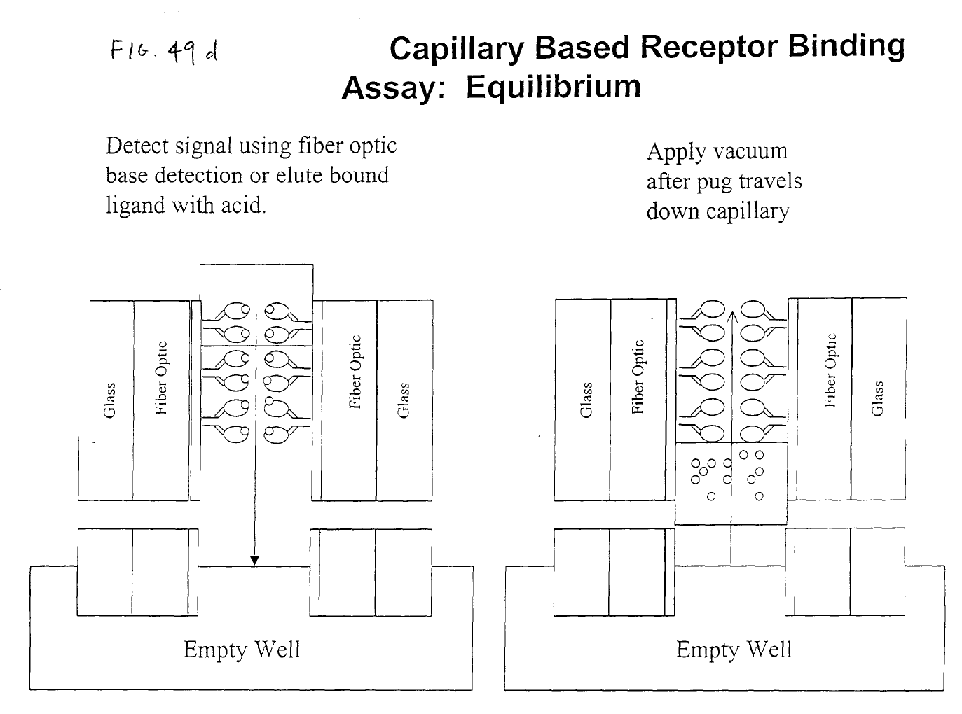

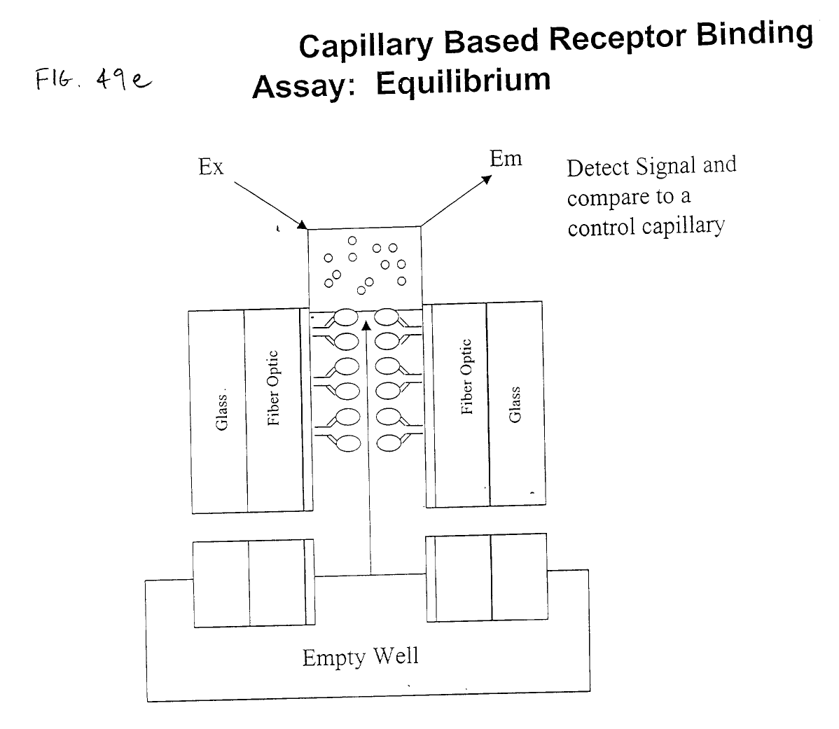

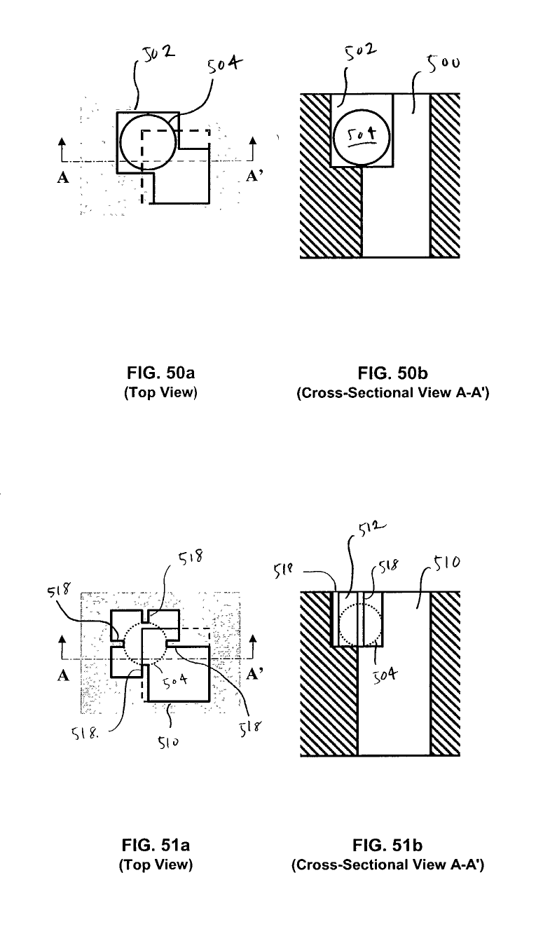

In one embodiment of the synthesis assay carrier, there exists an array of synthesis sites. Each site comprises a through-hole having a capture chamber. Two embodiments of through-holes with capture chambers are illustrated in FIGS. 50-51. In the embodiment shown in FIGS. 50a-50b, a through-hole 500 is provided with a capture chamber 502 alongside and partly overlapping with the through-hole 500. The capture chamber 502, also referred to as a “side pocket,” is in fluidic connection with the through-hole 500.

In some embodiments, the size of the opening of capture chamber 502 is designed to be slightly larger than the diameter of a selected polymer bead and the interior of the capture chamber 502 is designed to be able to just accommodate a defined number of beads, preferably a single bead 504.

In some embodiments, the size of the entrance and interior of the through-hole 500 can vary and may be smaller or larger than the diameter of the beads. As shown in FIG. 50a in a top view or horizontal cross-sectioned view, the entrance of the through-hole 500 is smaller than the diameter of the bead 504. This inhibits beads of this diameter from entering the through-hole 500. As shown by the dotted line 506, the cross-sectional width of the through-hole 500 can be increased at a location below the opening of the through-hole 500 to provide a larger volume for performing reactions within the through-hole 500. Alternatively, when the size of the through-hole 500 is larger than the bead size, any beads that are not captured by the capture chamber 502 can be flushed out of the through-hole 500. FIG. 50b shows a vertical cross-sectioned view of through-hole 500 taken along line A-A.

The component being captured by capture chamber 502 can be, for example, a bead 504 such as the polymer beads that are widely used for combinatorial chemistry synthesis, e.g., polyethylene beads. The diameter of the beads can be prefiltered to be within a defined range corresponding to the size of the capture chamber 502. The beads 504 can be randomly loaded onto the assay carrier. Because of the unique geometry of the carrier through-holes 500, given time and sufficient circulation of beads, each capture chamber 502 on the carrier will be loaded with a defined number of beads 504 corresponding to the size of the capture chamber 502. In some embodiments, after loading, the carrier is wetted with the entrance of the capture chamber 502 covered, causing the beads 504 to swell and lock themselves in the capture chamber 502. This procedure can be used to prevent the beads 504 from emerging from the capture chamber 502 during subsequent handling and/or mixing steps.

FIGS. 51a-51b illustrate an embodiment of a capture chamber 512 which facilitates efficient locking of the bead 504 while maximizing the exposure of bead surface to synthesis chemicals loaded into the through-hole 510. In this embodiment, multiple flanges 518 project inwardly from the walls of the capture chamber 512. In this embodiment, four flanges are used. When the captured bead 504 expands, these flanges press against the bead 504, thereby retaining it in the through-hole 512.

The size of the beads 504 can vary from, for example, 1 μm to 1000 μm in diameter. Correspondingly, the pitch of the through-hole assay sites can range from, for example, 2 μm to 1500 μm. As described in greater detail above, the material of the carrier can be, for example, silicon, metal, ceramic and suitable polymers, and the carrier can be fabricated using Deep Reactive Ion Etching (DRIE), molding, electroplating and electric discharge machining (EDM).

An alternative method to make the synthesis assay carrier is to use suitable plastics, such as polypropylene, as the substrate material, and then coating a layer of material of functional groups suitable for compound synthesis, such as polyethylene. In this case, bead loading can be omitted.

3. Additional Combinatorial Chemical Synthesis Applications

In accordance with another embodiment of the present invention, additional systems and methods for conducting highly parallel and miniaturized chemical synthesis are provided.

A first system and method of performing combinatorial chemical synthesis using three molecular groups, A, B and C, is described above with respect to FIGS. 34-38. In that embodiment, each molecular group can be one of N possible chemicals: A₁ through A_{N},B₁ through B_{N} and C₁ through C_{N}. It is therefore possible to make up to N³ different molecules, AᵢBⱼCₖ, using combinatorial chemistry methods, where i, j, k are integers in the range between 1 and N. This synthesis can also be performed using the synthesis carrier described above with respect to FIGS. 50-51.

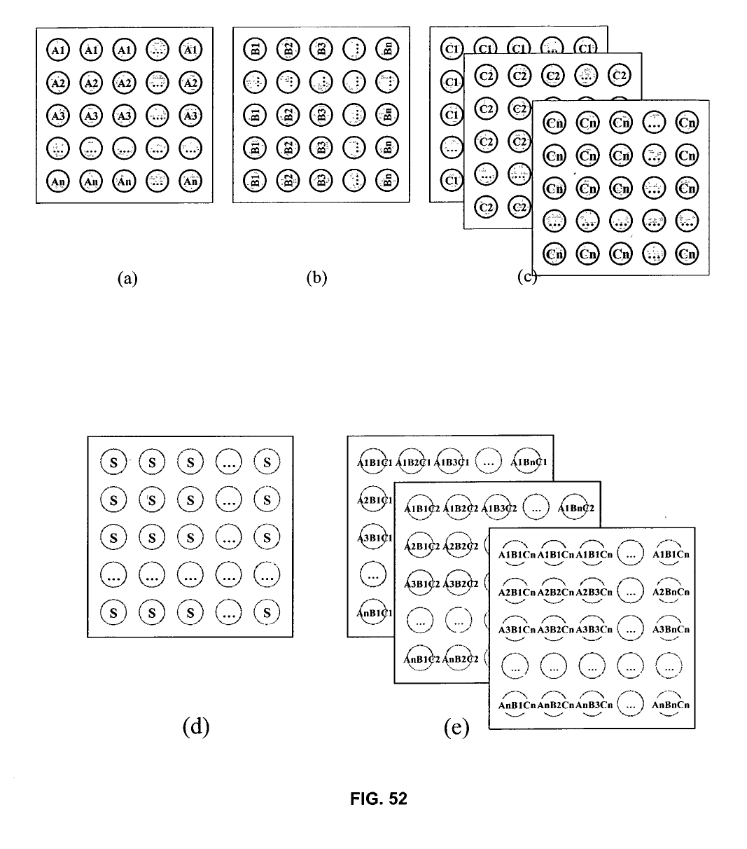

As described above with respect to FIGS. 34-38, the group A reagents are loaded onto N metering carriers (A Carriers), each carrier having a pattern shown in FIG. 52a wherein all of the through-holes in each row are loaded with a single one of the chemicals A₁ through A_{N}. The group B reagents are loaded onto N metering carriers (B Carriers) in a pattern shown in FIG. 52b, wherein all of the through-holes in each column are loaded with a single one of the chemicals B₁ through B_{N}. Next, the group C reagents are loaded onto N metering carriers (C Carriers) in a pattern shown in FIG. 52c, wherein all of the through-holes on each carrier are loaded with a single one of the chemicals C₁ through C_{N}.

Next, a bead or beads are loaded and locked into the capture chambers of the through-holes on another set of N assay carriers. A common scaffold is loaded into these N assay carriers to attach the scaffold onto the beads locked in each of the capture chamber assay sites, as shown in FIG. 52d.

Finally, the A, B and C Carriers are coupled onto the assay carriers containing the scaffold and beads to link Groups A, B and C to the scaffold. Accordingly, a library of N³ different molecules with AᵢBⱼCₖ combinations is generated on N assay carriers, as shown in FIG. 52e.

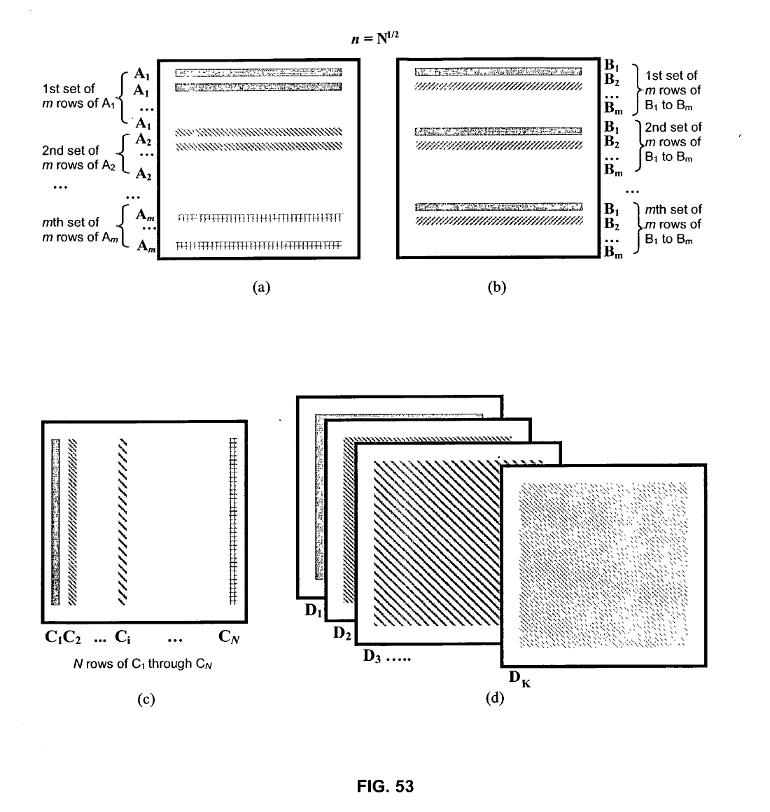

In another embodiment illustrated in FIG. 53, there are four molecule groups, A, B, C and D, available to be linked to a common scaffold. Groups A and B each have m different members, wherein m=\sqrt{N} or N^{1/2}. In other words, group A comprises m chemicals, A₁ through Aₘ, and group B comprises m chemicals, B₁ through Bₘ. Group C has N members and group D has P members. A library of P*N² compounds can be generated on P assay carriers, each assay carrier having an array of N×N through-holes.

First, the group A reagents are loaded onto P metering carriers (referred to as the “A Carriers”) in a pattern shown in FIG. 53a. Each of the A Carriers has m sets of group A chemicals. Each set of group A chemicals includes m rows of one of the chemicals A₁ through Aₘ. For example, the first set includes m rows, where all of the through-holes in each of the m rows is loaded with A₁. The second set also includes m rows, where all of the through-holes in each of the m rows is loaded with A₂. This continues to the mth set, which includes m rows, all of the through-holes in each of the m rows being loaded with Aₘ.

The group B reagents are loaded onto P metering carriers (referred to as the “B Carriers”) in a pattern shown in FIG. 53b. Each carrier has m sets of group B chemicals. However, in contrast with the A Carriers, each set of group B chemicals includes m rows of each of the chemicals B₁ through Bₘ. For example, the first set includes m rows, where all of the through-holes in the first row in the first set are loaded with B₁, all of the through-holes in the second row in the first set are loaded with B₂, and all of the through-holes in the mth row in the first set are loaded with Bₘ, The second set also includes m rows, where all of the through-holes in each of the m rows are loaded with chemicals B₁ through Bₘ, exactly as with the through-holes in the first set. This continues to the mth set, which includes m rows, and again all of the through-holes in each of the m rows are loaded with chemicals B₁ through Bₘ, as with the through-holes in the first set.

The group C reagents are loaded onto P metering carriers (referred to as the “C Carriers”) in a pattern shown in FIG. 53c. Here, each row on the carrier has one of the chemicals C₁ through C_{N}.

The group D reagents are loaded onto P metering carriers (referred to as the “D Carriers”) in a pattern shown in FIG. 53d. For the D Carriers, all of the through-holes for the first carrier are loaded with D₁, all of the through-holes for the second carrier are loaded with D₂, and so on, until the Pth carrier, which is loaded with D_{P}.

A common scaffold can be loaded onto P assay carriers to attach the scaffold onto beads locked at each through-hole assay site in the same way as described above with respect to FIG. 52.

The A, B, C and D Carriers can be coupled onto the scaffold assay carriers to link groups A, B, C, and D to the scaffold, thereby creating a library of P*N² different molecules with AᵢBⱼCₖDₗ on P assay carriers, using combinatorial chemistry methods. Here, i,j, k, and I are integers wherein i and j range between 1 and m, k ranges between 1 and N, and l ranges between 1 and P.

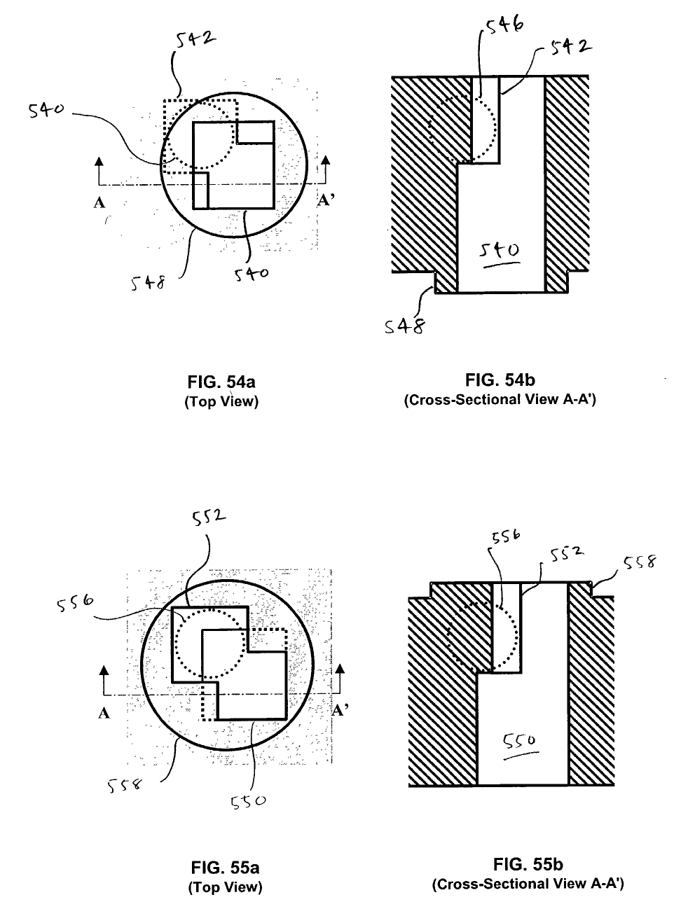

For many synthesis assays, the assay sites on the assay carrier are washed and cleared of liquid before the addition of the next reagent. In some embodiments, these assays can be carried out without the use of a metering carrier. All reagents can be loaded directly into the assay carrier. FIGS. 54-55 show embodiments of assay carriers having raised “island-like” surface features provided at one of the openings of the through-hole. These raised surface features can help to prevent fluid cross-talk between adjacent through-holes. In addition, the raised surface features may facilitate loading of multiple reagents using, for example, the MR loaders shown in FIGS. 35-36.

FIGS. 54a-54b show a through-hole 540 having a capture chamber 542 for capturing a bead 546 and a raised entrance 548 provided at an end of the through-hole 540 opposite the location of the capture chamber 542. The raised entrances 548 can be loaded using, for example, the MR loader 350 in FIGS. 35a-35b or the MR loading carrier 361 in FIGS. 36a-36b. Because the raised entrances 548 protrude beyond the rest of the surface of the carrier, the raised entrances 548 can more precisely mate with the channels 360 of the MR loading carrier 361 (FIG. 36) for MR loading.

FIGS. 55a-55b show another embodiment in which a through-hole 550 has a capture chamber 552 for capturing a bead 556 and a raised entrance 558 provided at the same end of the through-hole 540 as the capture chamber 552. The through-holes of this embodiment can be loaded as described above with respect to FIGS. 54a-54b.

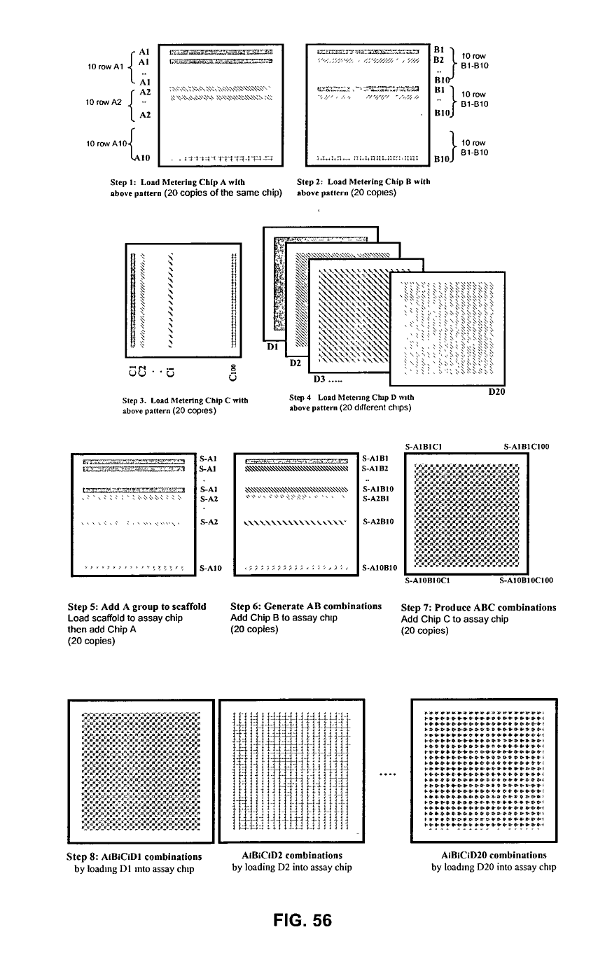

FIG. 56 illustrates one embodiment of a system with 10,000 metering reagent carriers and 10,000 assay carriers to synthesize 200,000 different chemicals. In this example, four different functional groups, A, B, C and D are added to a scaffold. Group A and B have 10 varieties each, C has 100 varieties, and D has 20 varities.

Using these chemical groups, a library of 200,000 different compounds can be generated using a “split-pool” strategy. The identity of the molecule on each bead can be decoded after the synthesis.

A bead as described above can be used as a substrate for compound attachment and can be loaded into a side pocket of a through-hole on the assay carrier as shown in FIGS. 50-51 and 54-55.

In accordance with the illustrated embodiment, an assay carrier has a single bead loaded into each of the 10,000 assay sites. The 200,000 different compounds can be synthesized on 20 assay carriers using the steps as illustrated in FIG. 56. The compound library can be used directly for screening to perform biochemical or cell assays without the need of bead picking and compound decoding.

While the above-described embodiments relate to the combinations of three or four reagents, other embodiments of the invention can be applied to greater or fewer reagents.

B. Addressable Pin System

In accordance with other aspects of the present invention, a metering reagent carrier incorporating addressable pins or arrays of pin sets is provided. As used herein, the term “pin” refers to a delivery system that can contain materials coated on the surface. The coating material can be a probe or substrate. The pins can be arranged in an array on a pin carrier such that it corresponds to and can be coupled with an assay carrier.

1. Pin Sets for Through-hole Array Systems

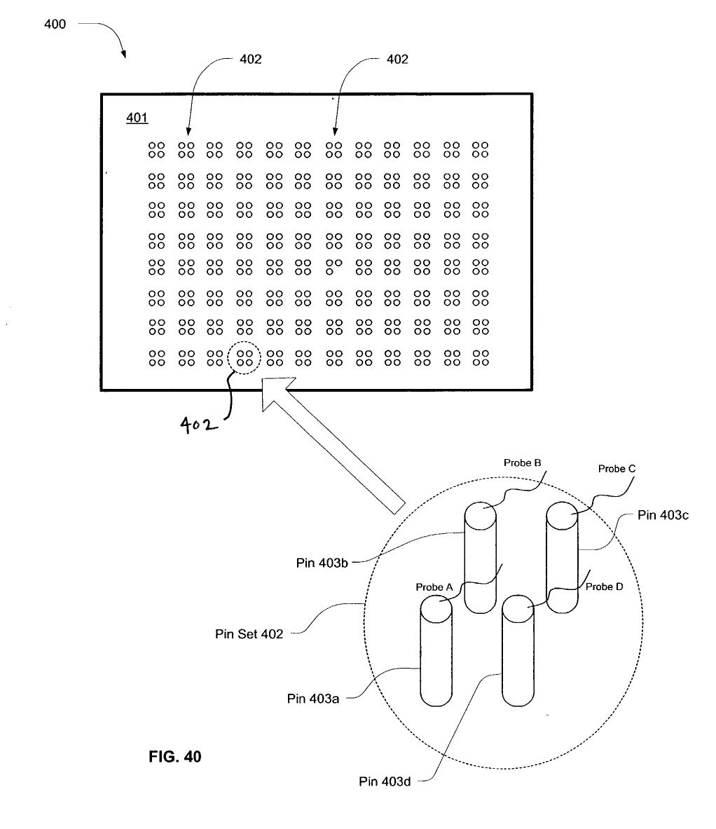

FIG. 40 shows a pin carrier 400 comprising a substrate 401 having an array of pin sets 402 provided thereon. The construction of the pin carrier 400 can be similar to that of the reagent metering carrier 30, except that in place of each pillar 32 on the reagent metering carrier 30, the pin carrier 400 has a pin set 402 comprising a plurality of individual pins 403.

In the embodiments described above with respect to FIGS. 1-5, a single pillar 32 on the reagent metering carrier 30 couples with a single through-hole 54 on the assay carrier 50. In the pin carrier 400 shown in FIG. 40, each pillar is replaced with a pin set 402 comprising four pins 403a-403d. The entire pin set 402 of four pins 403a-403d can be inserted into a single through-hole 54 on the assay carrier 50.

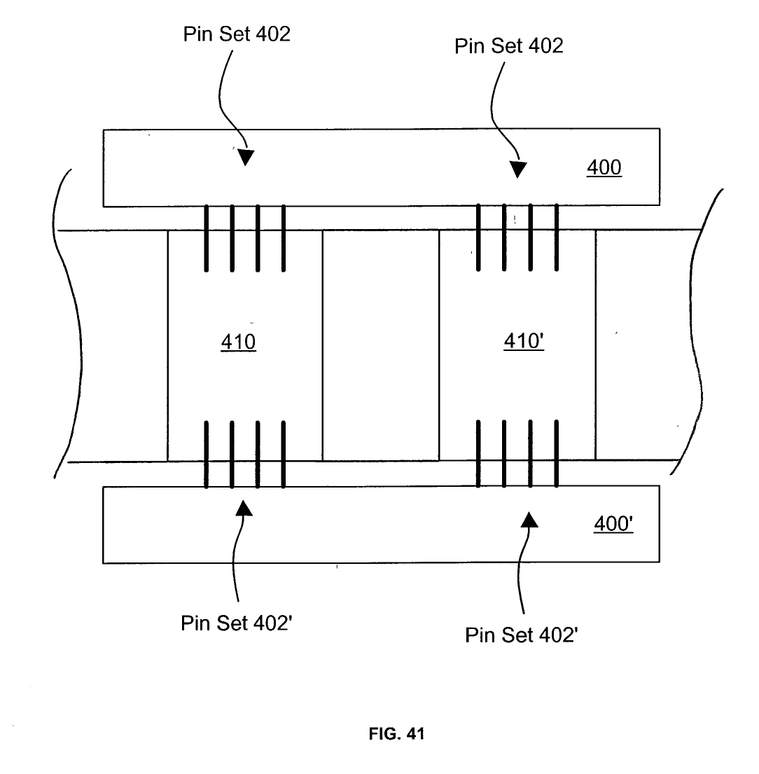

As shown in FIG. 41, a single assay carrier 50 can be simultaneously engaged by two pin carriers 400 and 400′. Through-hole 410 receives a first pin set 402 from pin carrier 400 above, and additionally receives a second pin set 402′ from pin carrier 400′ below. In the embodiment shown in FIG. 26, each pin set 402 comprises four pins, and each through-hole 410 is engaged with two pin sets 402 simultaneously. In other embodiments, the number of pins in each pin set can vary from one to as many as can fit into the through-hole 410. In addition, the pin carriers 400 and 400′ can engage assay carrier 50 simultaneously or in series with a delay or additional processing steps between each engagement.

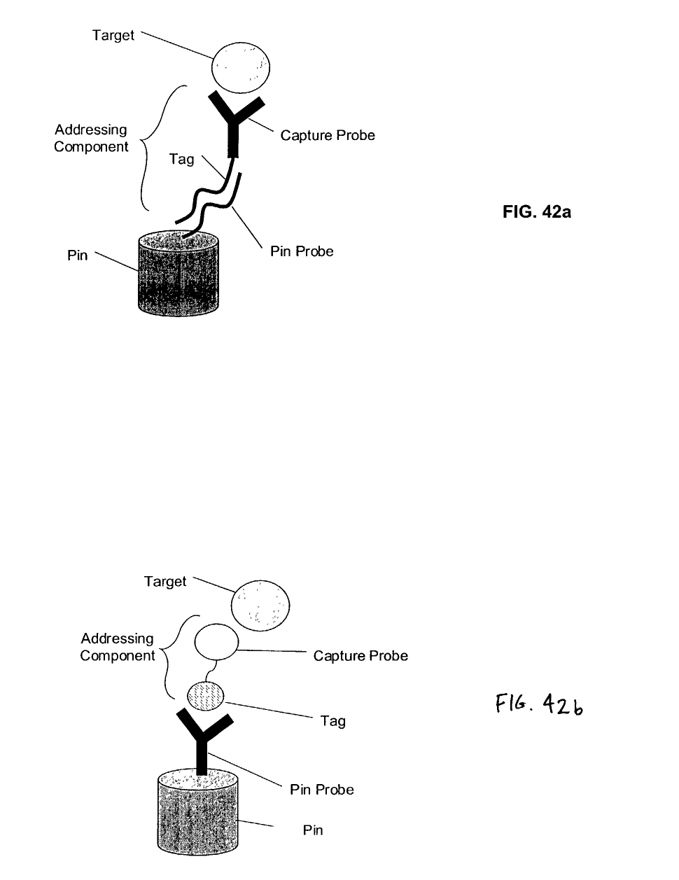

In accordance with embodiments of the present invention, each pin can be provided with a pin probe to capture a target located in a through-hole on the assay carrier, as shown in FIG. 42a. An addressing component can be used to connect the pin probe to the desired target. The addressing component may comprise a capture probe linked with a tag that specifically binds to the pin probe attached to the pin.

In the embodiment shown in FIG. 42a, the pin probe can be, for example, a DNA or RNA strand, the tag can be a complementary DNA or RNA strand, the capture probe can be an antibody, and the target is a cell or a biomolecule which can be captured by the antibody capture probe.

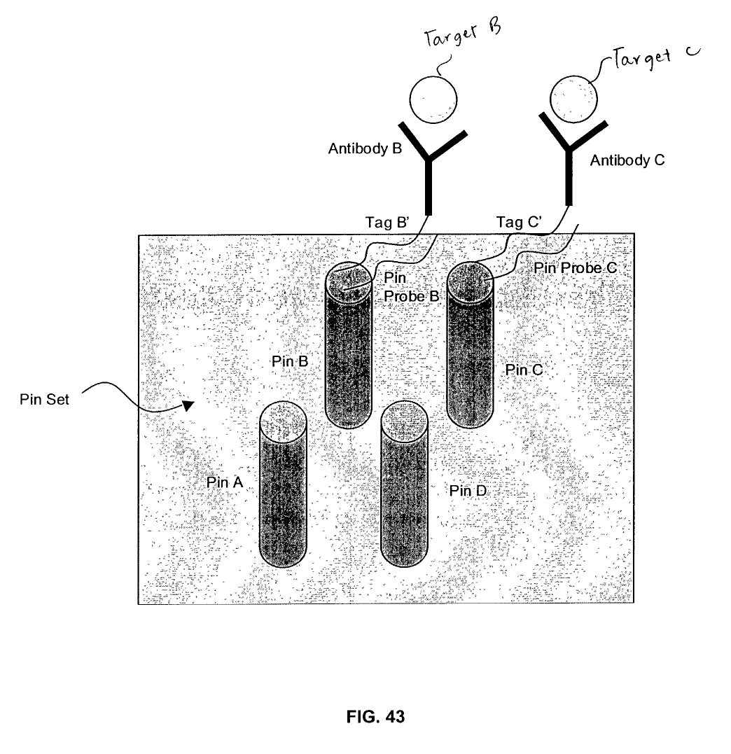

FIG. 43 shows a set of pins A, B, C, and D, which can be immersed in a single through-hole on an assay carrier. As shown in FIG. 43, pin B is provided with pin probe B, shown as a strand of DNA. The pin probe B can be attached to pin B by, for example, a covalent bond. Other attachment methods are possible, as would be understood by one of ordinary skill in the art.

The target for pin B is target B, which can be a cell in a solution contained within the through-hole. Also contained in the through-hole is the addressing component, comprising a capture probe (shown as antibody B) linked to a tag (shown as tag B′). The addressing component can be introduced into the solution using, for example, the reagent metering carrier described above. Once the addressing component is added, the target B will bond with the antibody B, which, in turn, is linked to tag B′. When pin B is inserted into the through-hole solution, probe B will bond with tag B′, thereby capturing target B onto pin B.

In accordance with another aspect of the invention, pin C is provided in the same pin set as pin B and is simultaneously immersed in the same through-hole as pin B. Pin C is provided with pin probe C. Pin probe C is complementary to tag C′, which is linked to a capture probe (shown as antibody C). Antibody C can be used to capture the target C. Therefore, as the pin set is immersed in the through-hole, target B and target C can be captured by pin B and pin C, respectively, using a single immersion step.

The above-described embodiments may permit high volume and robust manufacturing of a universal pin array while providing flexibility in designing the probes which are used to capture targets in the assay carrier through-hole. In particular, a pin carrier can be provided with a plurality of pin sets, each pin set comprising one or more pins, with each pin being provided with a pin probe. These pin probes can be used in a variety of different assays without further modification by selecting an appropriate addressing component to capture the desired target.

For example, a pin carrier may have a 100×100 array of pin sets, wherein each pin set comprises four pins A, B, C, and D. Each pin in the pin set can be provided with a unique pin probe A, B, C, and D, with each pin probe being complementary to a known tag A′, B′, C′, and D′, respectively. The four pin probes A, B, C, and D used by the pin carrier can be selected, attached to the pins, and delivered to a customer without regard to the particular assay for which the pin carrier is intended. When generating the desired assay, an appropriate capture probe which can capture the desired target is provided with one of the known tags A′, B′, C′, and D′. The pins on the already-prepared pin carrier can then be used to capture any desired target, thereby providing great flexibility in assay design using generically-treated pins on the pin carrier. Hence, any particular assay can be readily designed without optimization during the manufacturing of the multi-pin array.

This method can be demonstrated by the following example. Each pin in a pin set is coated with a pin probe comprising, for example, a unique DNA sequence. When using the arrangement shown in FIG. 41 in which each through-hole is engaged with two pin sets (one from above and one from below), and each pin set comprises four pins, eight unique DNA sequences are provided for each through-hole. Eight addressing components corresponding to the eight pin probes are provided, each addressing component comprising a tag of complementary DNA or RNA sequences coupled to a unique antibody capture probe. These antibodies can be used to bind to eight unique targets. In an appropriate solution, the tags will bind to a single one of the eight pins provided with a complementary DNA sequence. This pin together with the addressing component can be used as a “molecular fishing” system to select and separate a specific target within a complex mixture in assay carrier.

For example, this system can be used to detect the presence of cytokines in human sera. Alternatively, it can be use to retrieve several targets in a biochemical assay such as proteinases or kinases.

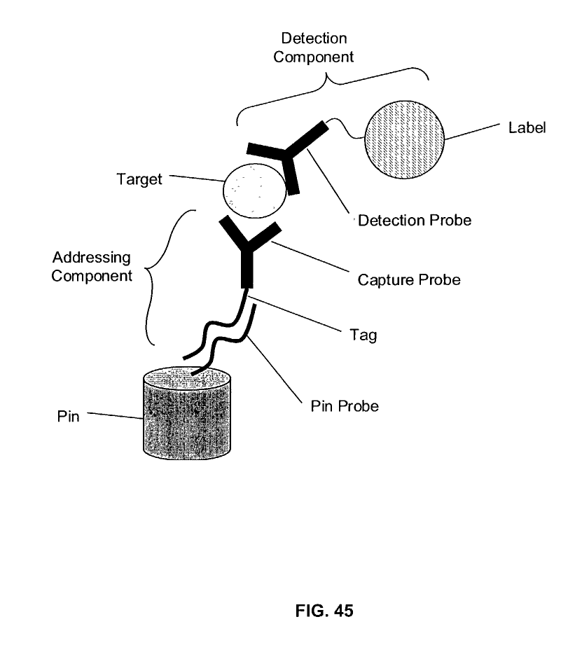

After the target has been captured by the appropriate pin, the pin can then be moved to a wash station to remove non-specific materials. To detect the presence of the target cell, a detection component can be used, as shown in FIG. 45. In the embodiment shown in FIG. 45, the pin having the pin probe, tag, capture probe, and target attached thereto is placed into a well containing a detection component. The detection component may comprise, for example, a detection probe, such as another antibody for bonding with the target, and a label linked to the detection probe. The label can be a fluorescent dye such as, for example phycoerythrin. When the pin is inserted into the well containing the detection component, the detection probe will bond to the target, thereby enabling detection of the presence of the target.

The pin array may then be removed and dipped into wash solution and each pin array can be imaged to determine the signal on each well. For a 10,000 well assay carrier having eight pins inserted into each well, 80,000 data points can be obtained.

The use of addressable probes per pin can be useful for manufacturing since the deposition of the pin probe onto the pin need not be varied in order to be used in different assays. This can permit robust manufacturing processes by keeping the pin deposition process the same every time despite its use in different assays. In addition, this system can permit an end-user to design their own assay by providing a customized addressing component, such as a basic DNA labeling reagent to their specific probe.

Embodiments of the present invention can also include nucleic acid probes which may be more robust with respect to storage and stability. However, other addressable methods can be used. For example, each pin can be coated with a pin probe such as an antibody, single chain antibody variable region fragment (scFv), aptamer, or other high specific affinity probes for specific labels. Each capture probe can be incorporated with a specific label. For example, a small unique peptide tag recognized by a unique antibody, aptamer, or scFV, etc. It may be desirable to have the binding affinity be high and specific. One embodiment is shown in FIG. 42b, which has a pin probe comprising an antibody. The antibody pin probe captures an addressing component comprising a protein capture probe linked to a peptide tag. The type of capture pin can be used to capture a target such as a biomolecule or a cell.

Two examples of how the addressable probes described above can be used are provided below. In a first case, the addressable pin array is placed into a buffer containing a complementary addressing component mixture and is incubated to permit specific binding of the tags to the capture probes. Then the pin array can be used to capture the target. In a second case, an addressing component mixture is added to the sample, and then the pin array is placed into the sample to extract the addressing component and the target.

In accordance with other embodiments of the present invention, an addressable pin system can be used to accurately deliver a single cell to each through-hole in an assay carrier. A single cell from a heterogeneous or homogenous population can be captured and delivered to a through-hole in an assay carrier. In some embodiments, the cells can be, for example, about 8-40 μm in diameter. In other embodiments, the cells can be smaller or larger. Several cell-based assay can be performed such as drug compound effects on ADME/Tox, GCPR and signal transduction pathway.

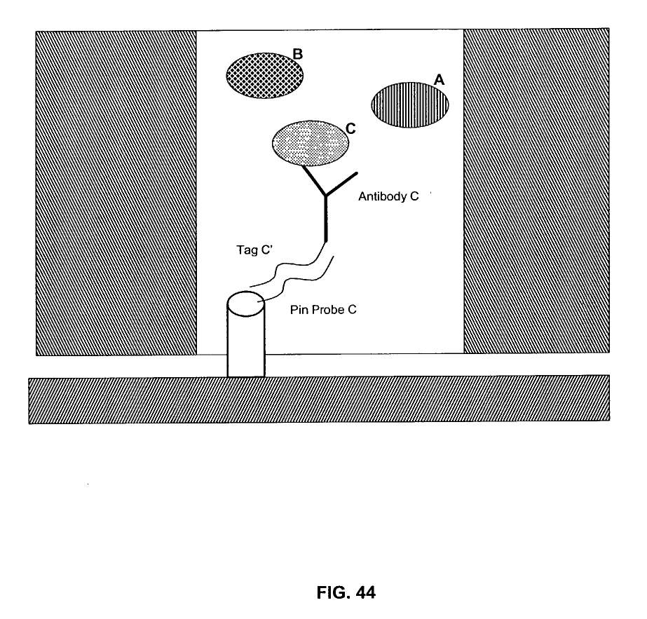

For example, in FIG. 44, capture probe C can be an antibody specific for a cancer marker, and the capture probe C antibodies can be used to capture a single cell from a mixed cell population, such as blood. Alternatively, because of the small pin size, the capture probe C antibodies can be used to capture a single hepatocyte.

In one embodiment, pin “A” which has been used to capture a single cell is placed in an assay carrier through-hole containing a lysis buffer that disrupts the cell membrane. This disruption releases the cell contents, which are then interrogated by pin “B” containing an antibody capture probe bound to specific target from a cell lysate. Each pin “B” contains a capture probe for an unique target within a cell. The pin “B” array is then moved to another assay carrier through-hole which contains a reagent for signal generation and analysis.

In another case, a cell captured by the target probe attached to the pin can be fixed. An antibody probe can be used to quantitate expression of protein inside the fixed cell.

Embodiments of the addressable pin system can have three utilities: (1) the use of multiple pins to perform multiplex micro-well heterogeneous assays; (2) the use of addressable pins for delivering and capturing a single cell for cell-based analysis; and (3) the simplicity of manufacturing multiple or single addressable pins with universal pin probes to a corresponding label on target probes.

2. Pin Signal Amplification

Another aspect of the addressable pin system for heterogeneous liquid assays involves the amplification of the observed signal. Below are some of methods that are suitable for use with the addressable pin system described above.

First, a detection component can be labeled with enzyme such as horseradish peroxidase (HRP) or alkaline phosphatase (AP) and various colormetric, fluorescent, and chemiluminescent substrates can be used. Chemiluminescent substrate from Tropix, Inc. such as CSPD® with an Emerald enhancer can extend the length and intensity of the signal.

Second, a detection component can be labeled with DNA which can be amplified with PCR (immuno-PCR) or T7 RNA polymersase (IDAT).

Third, a detection component can be labeled with long length of single-stranded DNA. A set of short complementary probes labeled with fluorescent, enzyme or biotin is used to amplify the signal. Signal enhancement can be significant by coupling with chemiluminscent substrates. Branch oligo amplification system is an extreme case.

Two detection components can be used to detect two different sites on the target. The detection component can contain oligo which can be ligated and amplified using real-time PCR reader. The detection component can be aptamer, antibodies and others. This may provide extreme sensitivity (more than Immuno-PCR) and the assay can be done homogeneously. Like the immuno-PCR, temperature cycling and real-time PCR in micro-scale should be controlled.

In summary, embodiments of the present invention can be used as follows: (1) multiplex pins per through-hole well, which can enable high-throughput screening of compounds using multi-plex quantitation of enzyme, sandwich immunoassay, DNA and protein-to-protein assay per well; (2) addressable pin systems for delivering one or more cells can be used in any cell based assay; and (3) addressable pins coated with a self-assembly probe can simplify manufacturing by providing a universal pin array. These systems can provide flexibility in assay development by providing generic pin probes which can be used with customizable addressing component. The use of amplification described above can be applied to many assay formats, such as, for example, enzymatic, nucleic acid, protein-to-protein, DNA-to-protein and sandwich antibodies.

### V. EXAMPLES

By employing various combinations of the above-described methods, a variety of liquid based assays, including homogeneous and heterogeneous assays, can be performed in accordance with embodiments of the present invention. The following are some specific examples of embodiments of the present invention as used to perform assays in a highly parallel fashion.

### Example 1

Kinase Assays

Kinases are important targets for new drug development. There are two major classes of protein kinases: the tyrosine kinases and the serine/threonine kinases. Some of the most widely used assay formats for these classes of enzymes include the scintillation proximity assays (which use radioactivity), fluorescence polarization using phospho-specific antibodies or immobilized metal ions, or homogeneous time resolved fluorescence energy transfer methods, which also use labeled antibodies. High affinity anti-phosphotyrosine antibodies are commercially available and universally applicable for all tyrosine kinases, however, similar universal antibodies do not exist for serine/threonine kinases. At the same time, serine/threonine kinases are much more widespread and thus new or improved methods for assaying their activities may be of particular importance.

The following assay for kinase activity in accordance with embodiments of the present invention can serve as an example. For screening kinase inhibitors, kinase assays can be performed by first mixing the kinase of interest with the compounds to be tested as kinase inhibitors, a suitable biotinylated peptide substrate, and all other reagents used for the enzymatic phosphorylation in the assay carrier. The compounds, peptide substrate, and any other reagents can be added to the through-holes of the assay carrier using a pin carrier. After a suitable incubation period, a capture carrier is inserted into the assay carrier to capture all biotinylated molecules, both phosphorylated and non-phosphorylated. A suitable capture reagent in this case is streptavidin. The capture carrier may have protrusions onto which streptavidin is immobilized. Following the capture step, the capture module is washed with a suitable washing buffer. This washing step can be simple and may consist of simply passing a stream of the wash buffer over the capture carrier. The capture carrier is then inserted into a third carrier containing a solution of a labeled detection reagent. This can be, for example, an anti-phosphotyrosine antibody linked to an enzyme such as alkaline phosphatase if a tyrosine kinase activity is tested. To detect the presence of kinase activity on the surface of the capture carrier, the capture carrier is finally inserted into a solution containing a suitable substrate for detecting the activity of the antibody-linked enzyme, such as, for example, a fluorogenic alkaline phosphatase substrate.

As an alternative to the enzyme linked antibody, other embodiments of the present invention can use a polycationic molecule immobilized onto the surface of the capture carrier. Examples of suitable polycationic polymers are polyarginine, polylysine, polyethyleneimine, and numerous other similar reagents that are either commercially available or can be easily prepared. As the kinase substrate and product molecules typically differ in charge, conditions can usually be identified where the more negatively charged reaction product will bind preferentially to the charge-modified surface than the less negatively charged substrate. To achieve better binding selectivity, an additional washing step can be added using a solution of suitable ionic strength. In one embodiment, the kinase substrate used is a fluorescently labeled peptide. The detection of the captured product can be carried out either on the capture carrier directly, or it can be released into an additional detection module by treatment with a high ionic strength solution, or alternatively by other suitable chemical or enzymatic treatments.

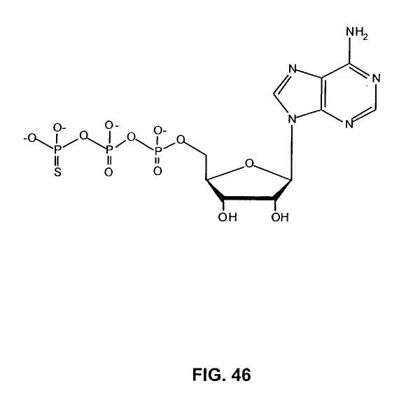

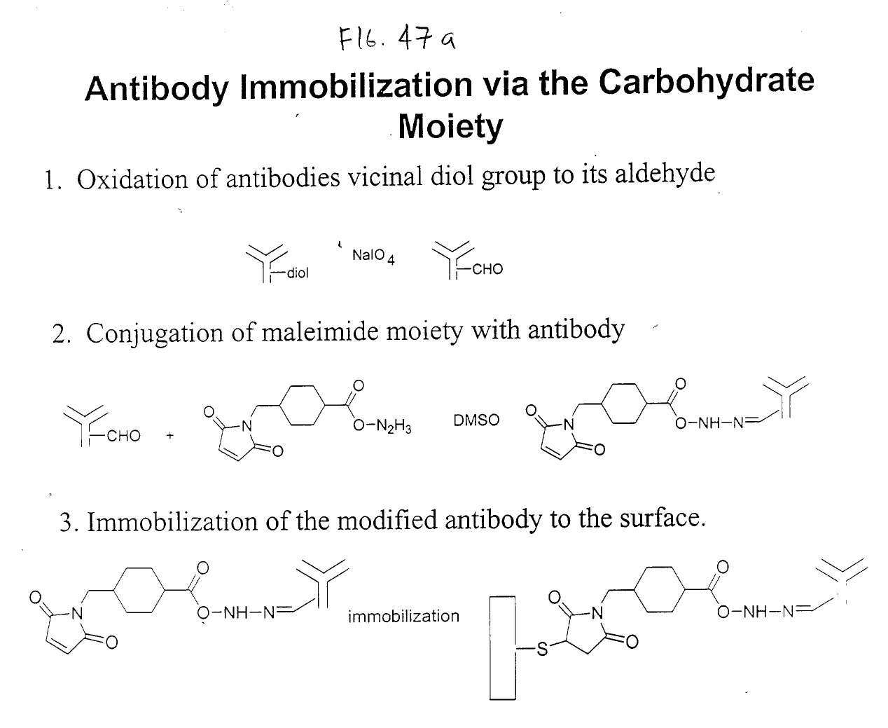

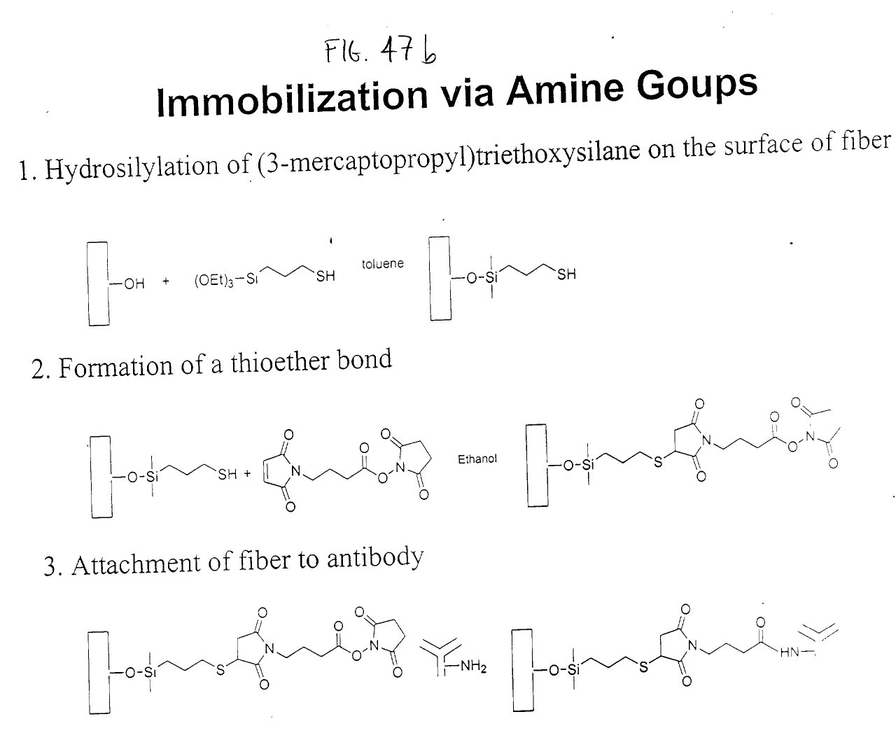

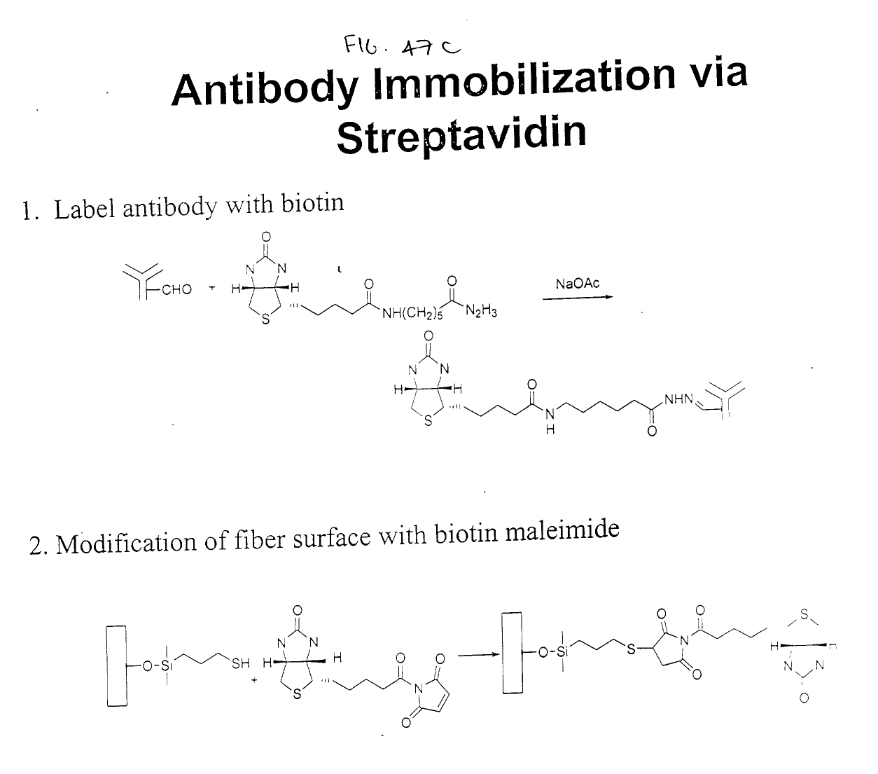

Yet another kinase assay utilizes an ATP analog, ATPγS (shown in FIG. 46). When a biotinylated kinase substrate is incubated with a target kinase in the presence of this ATP analog, the reaction product will incorporate a thiophosphate group. The substrate containing the thiophosphate group can be selectively modified by reaction with sulfur-specific reagents such as iodo- or bromo-acetamides, maleimides, mixed disulfides and other derivatives of fluorescent dyes. Thus, the reaction product can be captured on the streptavidin coated surface of a capture carrier module and then transferred to a modification carrier, where the captured thiophosphorylated product is allowed to react with one of the above-mentioned sulfur-reactive dye derivatives. The reaction product can then be read either directly on the capture carrier or after transferred to another detection carrier module and released in solution.

Since the amount of fluorescent dye molecules that can be captured onto the streptavidin-coated capture carrier is very low, the detection of the captured fluorescent dye molecules may be difficult. Therefore, in a different version of the same assay, a signal amplification step employing an enzyme-linked antibody can be used. In this case, the thiophosphorylated kinase reaction product will be captured onto a streptavidin coated capture carrier as before, but the modification reaction will be carried out with a reagent of the general structure R—Y, where Y is a thiol-reactive group such as a halo acetate, a maleimide, a disulfide, a Michael acceptor such as an acrylic acid derivative, and others, and where R can be any group that can be specifically recognized and bound by a specific antibody. Examples for suitable R groups include biotin, dinitrophenol, dioxygenin, and any number of other haptens. Following the sulfur-modification reaction, the capture carrier will be contacted with a solution containing a suitable enzyme-linked antibody against the R group, and finally the binding of this antibody will be revealed by contacting the capture carrier with a solution of a suitable substrate for the enzyme that is attached to the antibody. Typical enzymes that can be used in this enzyme-amplification system include alkaline phosphatase, horseradish peroxidase, galactosidase, and others.

### Example 2

Phosphatase Assays

The following assay for phosphatase activity can serve as an example for as assay carried out in the “capture and release” format. Phosphatases are enzymes that remove a phosphate group from tyrosine, serine or threonine residues of proteins and peptides. They constitute another important class of drug targets.

According to embodiments of the present invention, tyrosine phosphatases can be assayed by using target-specific peptides that contain a biotin residue at one end, a fluorescent dye at the other, and a phosphotyrosine residue within the peptide sequence. First, the through-holes of an assay carrier are filled with a solution containing the peptide that contains a biotin at one end and a fluorescent dye such as fluorescein at the other end. In addition, the peptide contains a phosphorylated tyrosine residue and additional amino acids that allow its specific binding by a tyrosine phosphatase to be tested. The sample to be tested for tyrosine phosphatase activity can be loaded into the through-holes of the assay carrier using a reagent metering carrier. Following an incubation period, during which some of the phosphorylated substrate molecules are enzymatically dephosphorylated generating peptide molecules containing an unmodified tyrosine residue, the mixture of biotinylated substrate and product molecules is captured on a streptavidin-coated capture carrier. The enzymatic dephosphorylation will result in the formation of the dephosphorylated version of the same peptide. The substrate/product mixture can now be captured onto a streptavidin-coated capture module which is then transferred to another assay carrier containing a solution of chymotrypsin. This protease will selectively hydrolyze the dephosphorylated peptide, while leaving the phosphotyrosine containing substrate intact. The released peptide fragment, containing the fluorescent dye, can subsequently be detected on the assay carrier by measuring the fluorescent signal. The absence of a phosphatase inhibitor in the original phosphatase reaction will result in a relatively high fluorescence intensity in the corresponding well, whereas the presence of a phosphatase inhibitor will be manifested by a low fluorescent signal. Chymotrypsin is a protease that may be well suited for this assay, as it has a preference for a Tyr residue in the P1 substrate position. For serine/threonine phosphatases, other suitable enzymes can be identified or developed by genetic engineering.

### Example 3

Protease Assays

Protease assays are very often carried out using relatively short peptide substrates that are specifically recognized and cleaved by the target protease. Currently, one widely used format utilizes the dual labeling of the protease substrate with a fluorescent dye and a quencher. Positioning of the dye and the quencher is optimized to achieve good performance in these types of assays. A different version of such assays has been described, which relies on the use of peptide substrates labeled at one end with biotin and at the other end with a fluorescent dye. Streptavidin binding is used at the end of the assay to bind all biotinylated molecules, and the extent of proteolytic cleavage is estimated by measuring the fluorescence polarization of the dye. Similar protease assays can be performed in a heterogeneous format by capturing the biotinylated molecules onto a streptavidin-coated capture carrier module and subsequently measuring the signal of the captured fluorescent dyes as described above. In the absence of protease inhibitors, a significant fraction of the biotinylated substrate molecules will be cleaved and, correspondingly, a relatively low fluorescent signal will be detected after the capture step. If the compound tested has inhibitory activity against the protease of interest, then a smaller fraction of the substrate molecules will be cleaved, and a higher fluorescent signal will be detected after the capture. In comparison to the homogeneous fluorescence polarization based format, this new format can offer an improved sensitivity and increased dynamic range. Just as in the case of kinase reactions, it is possible to include an enzyme-linked signal amplification step in order to increase the signal. This could be achieved by replacing the fluorescent dye from one end of the biotinylated protease substrate molecule by a hapten such as dinitrophenol, fluorescein, etc., and using an enzyme-linked antibody against that hapten after the capture step.

### Example 4

Protein Binding Assays

Protein binding assays can be accommodated by the assay format described herein. As an example, to study the effect of various test compounds on the binding interaction of two proteins, one of the proteins can be biotinylated and the other protein labeled with either an enzyme such as alkaline phosphatase or an otherwise detectable moiety, such as a fluorescent dye. If the compound tested does not interfere with the binding interaction of these two macromolecules, then after a capture step on a streptavidin coated carrier, the presence of the enzymatic or fluorescent marker on the second protein can be detected. To increase the sensitivity of the detection step, an enzyme-linked signal amplification system can be used here in the same way as described above for the kinase and protease assay systems.

### Example 5

Receptor Binding Assays

A high throughput screen for a receptor binding assay can be performed in accordance with aspects of the present invention. The high throughput screen may involve (i) strategies for immobilizing a probe molecule (antibody, receptor) within a capillary and (ii) an approach for carrying out the assay to measure binding kinetics.

1. Antibody Immobilization Strategies

There are several strategies for immobilizing an antibody on the inner wall of a capillary:

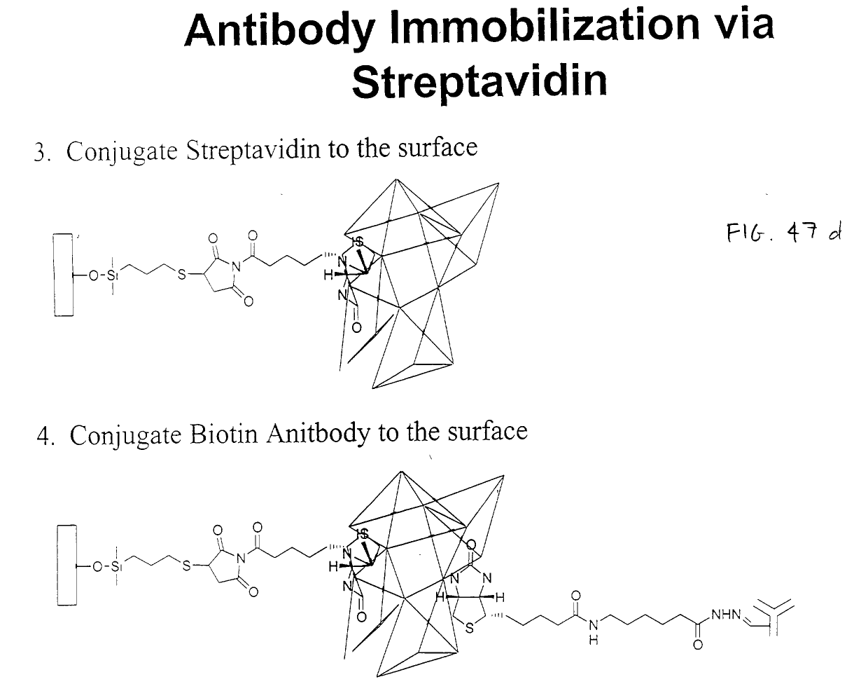

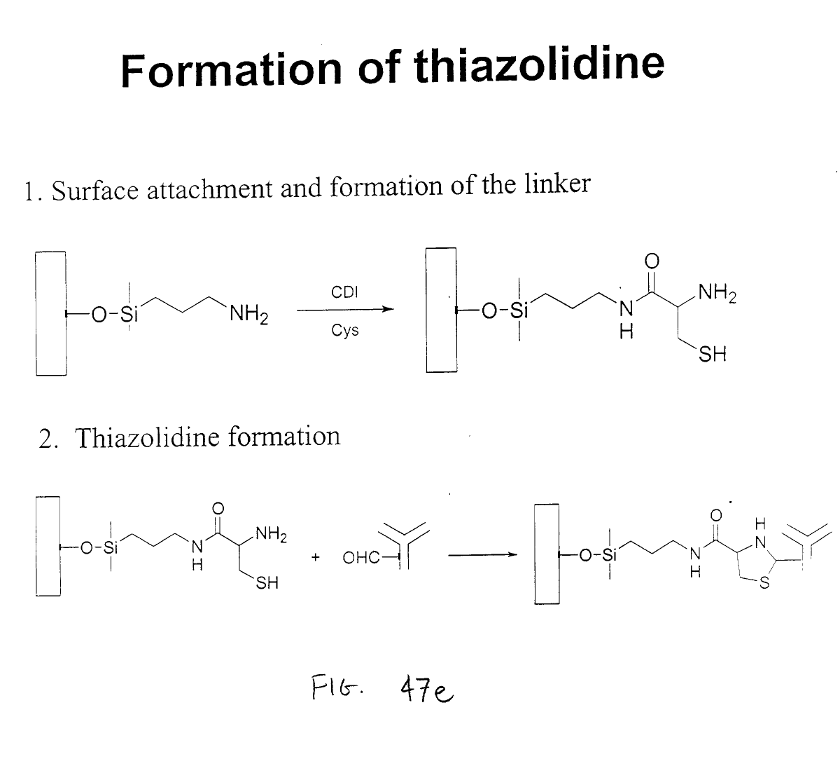

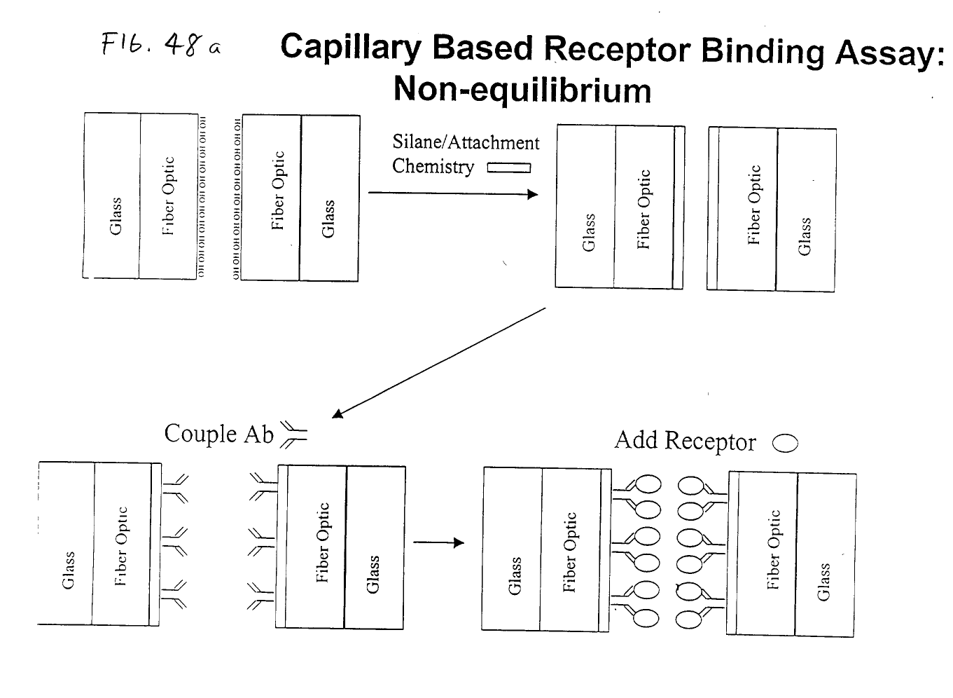

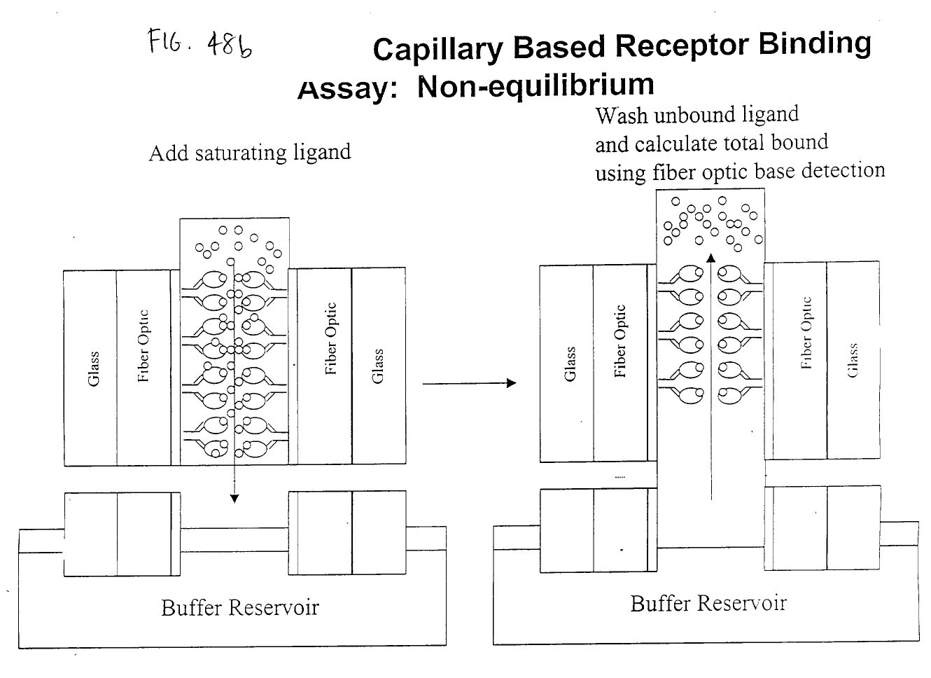

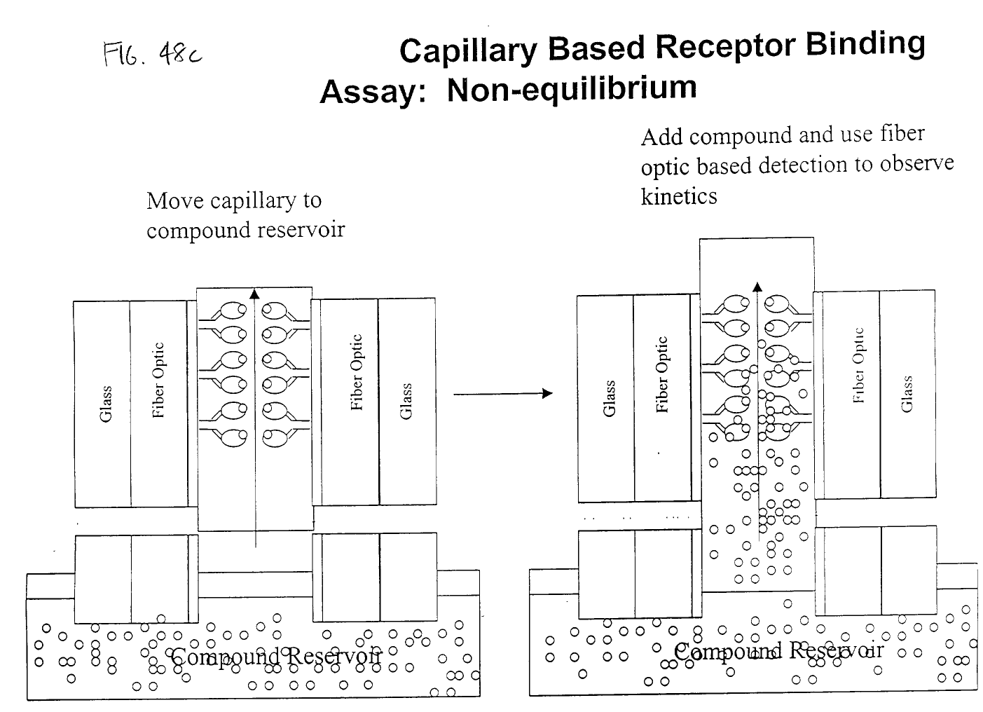

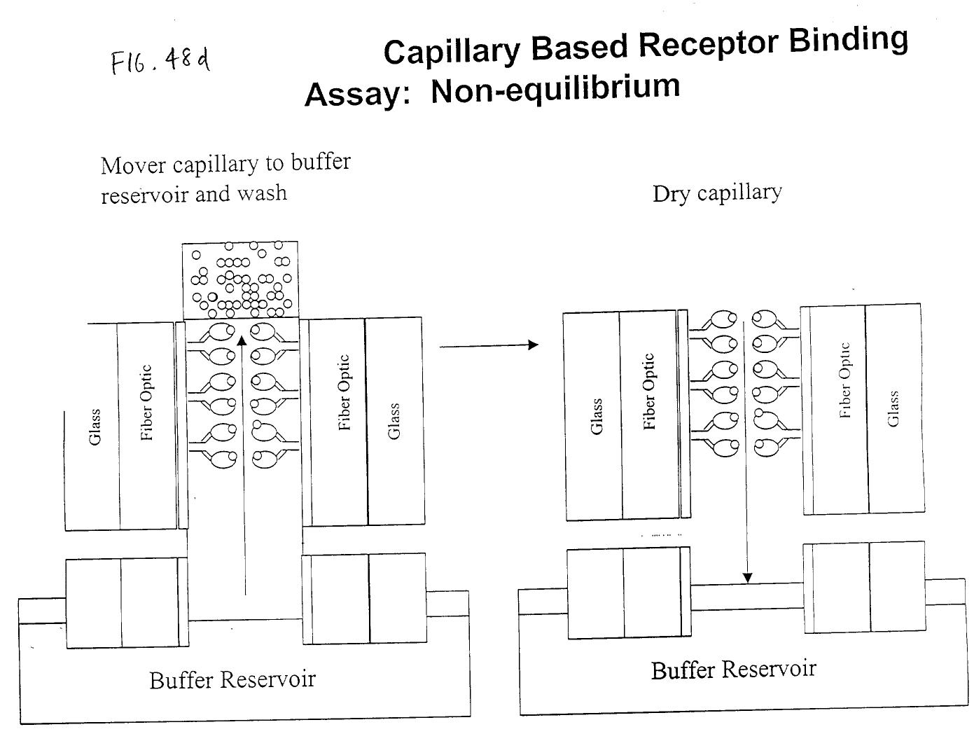

A first embodiment shown in FIG. 47a uses immobilization via the carbohydrate moiety. The process involves oxidation of antibody's vicinal diol group to its aldehyde followed by conjugation of a maleimide moiety with the antibody and immobilization of the modified antibody to the surface.

FIG. 47b illustrates immobilization via amine groups by hydrosilylation of (3-mercaptopropyl) triethoxysilane on the surface of fiber followed by formation of a thioether bond and then attachment of fiber to antibody.

FIGS. 47c and 47d illustrate immobilization via avidin-biotin binding. The antibody is labeled with biotin. The fiber surface is modified with biotin maleimide. Then Streptavidin is conjugated to the surface followed by conjugation of biotinylated antibody to the surface.

FIG. 47e illustrates immobilization via surface attachment, linker formation and thiazolidine formation.

2. Receptor Binding Assay under Non-Equilibrium Conditions

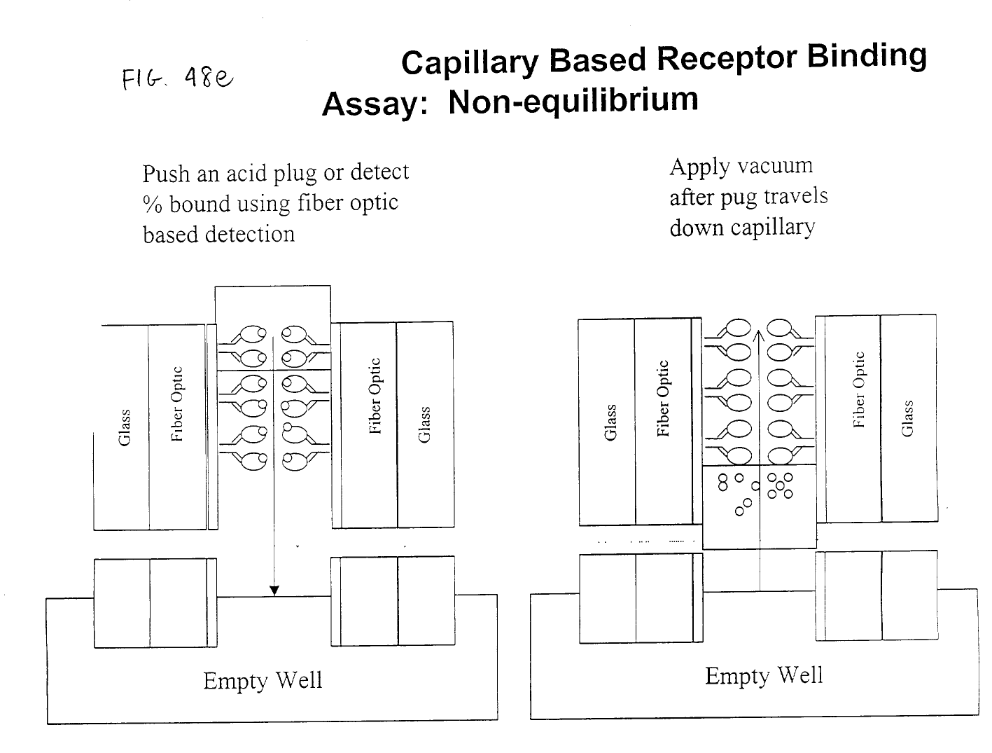

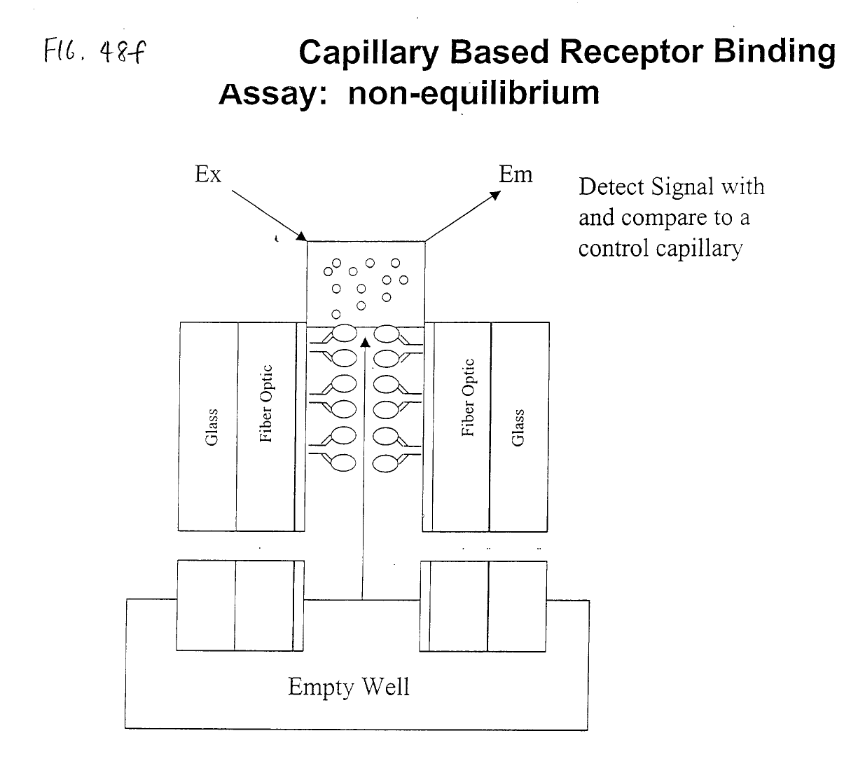

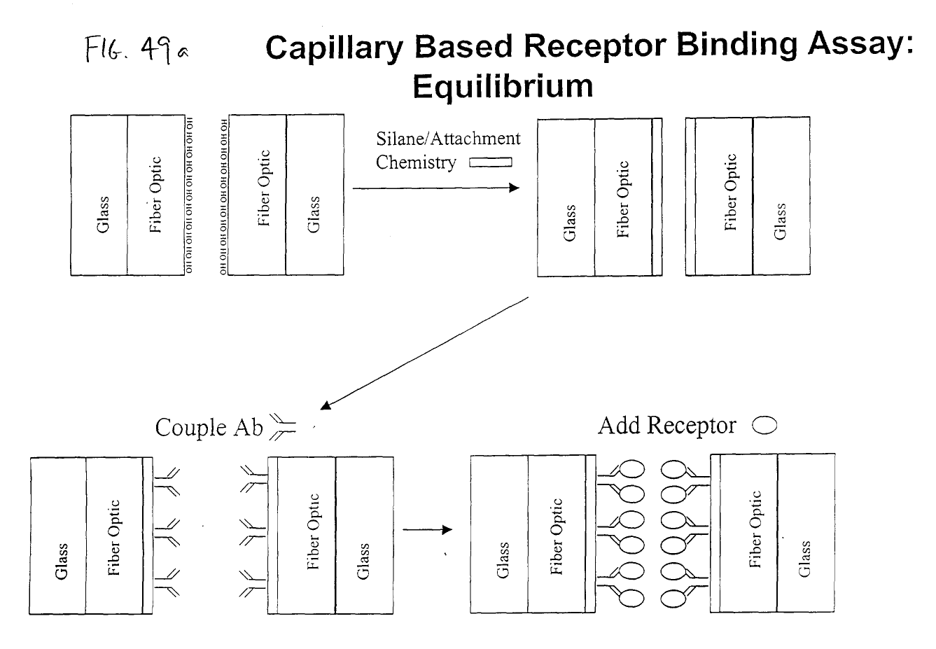

FIGS. 48a-48f schematically illustrate a non-equilibrium receptor binding assay within a fiber optic capillary.

The interior wall of a capillary is silanized and coupled to an anti-receptor antibody. The receptor is then immobilized on the capillary walls by being bound to the antibodies. A saturating amount of ligand specific for the receptor is added and following incubation, unbound ligand is washed away and total bound ligand is calculated using fiber optic based detection methods. The capillary array is then transferred to a reservoir containing a compound of interest. Following addition of the compound, fiber optics based detection methods are used to follow the kinetics of competitive binding between the ligand and the compound to the receptor. The capillary array is then moved to a buffer reservoir and unbound ligand and compound washed away. An acid plug is then introduced into the dry capillary to displace the bound ligand and compound. Once the acid plug has contacted the receptor molecules, it is extruded by a negative pressure and the signal generated by the ligand and compound mixture in the acid plug is measured against a control which was not challenged with the compound. The signal may be generated by the ligand, or the compound or both, but preferably the ligand. When the ligand carried the fluorescence label, the detectable fluorescence decreases over time as unlabeled compound displaces the ligand. Detection of the kinetics of this process allows the avoidance of false positive data.

3. Receptor Binding Assay under Equilibrium Conditions

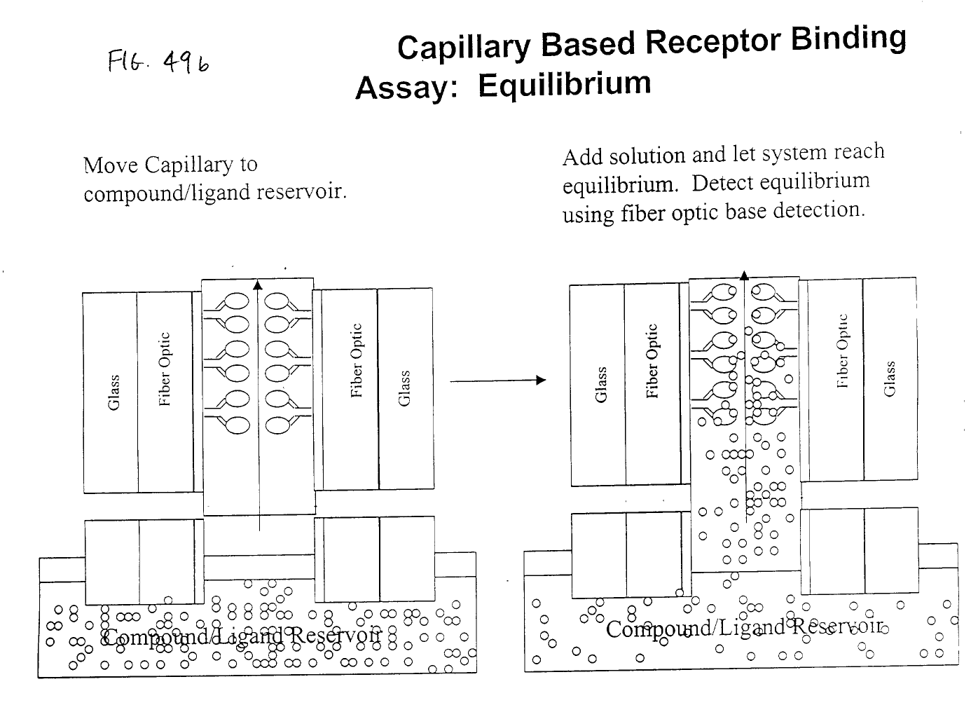

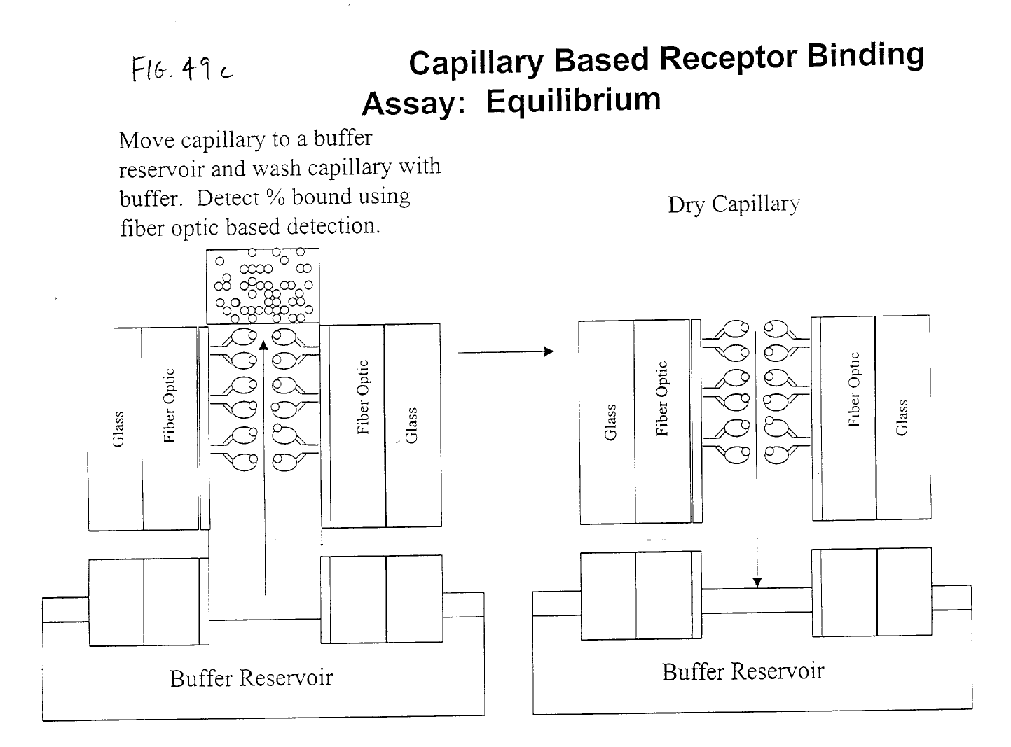

FIGS. 49a-49e schematically illustrate a receptor binding assay within a fiber optic capillary under equilibrium conditions.

The interior wall of a capillary is silanized and coupled to an anti-receptor antibody. The receptor is then immobilized on the capillary walls by being bound to the antibodies. The capillary array is transferred to a reservoir containing both ligand and compound. Sufficient ligand/compound solution is added and incubated with the receptors to reach equilibrium. The attainment of equilibrium is detected by fiber optics based detection. The capillary array is then transferred to a buffer reservoir and washed with the buffer to remove unbound ligand and compound. Percentage of ligand and/or compound bound is detected by fiber optics based detection. An acid plug is then introduced into the dry capillary to displace the bound ligand and compound. Once the acid plug has contacted the receptor molecules, it is extruded by a negative pressure and the signal generated by the ligand and compound mixture in the acid plug is measured against a control which was not challenged with the compound. The signal may be generated by the ligand, or the compound or both, but preferably the ligand. Since this process determines the end-point of the reaction, it is not absolutely necessary to use fiber optic capillaries.

### Example 6

Antibacterial Screening Assays

In accordance with embodiments of the present invention, high throughput antibacterial screening systems and methods are provided. This may be accomplished by first depositing a heated agar in liquid form as a UR into the capillaries of the assay carrier, and then adding a liquid media (broth) to the agar-filled capillaries using the reagent metering carrier. The bacteria of the desired strain can be cultured and grown within the assay carrier capillaries. Alternatively, the desired bacterial microbes can be deposited into the interior of the capillaries in an assay carrier by flooding or by using the pillars on a metering carrier.

Next, the suspected antimicrobial compounds can be loaded onto the pillars of a reagent metering carrier and then transferred to the capillaries of the assay carrier containing the cultured microbes. Following the diffusion of the compounds through the microbe solution, the capillaries can be examined to determine whether any zones of inhibition have formed within each capillary. Optical inspection can be used to determine the existence and extent of antibacterial activity.

While the discussion above related to the use of the a high throughput screening system for antibacterial screening assays, it will be understood that antifungal assays may also be performed using similar techniques as those applied for the antifungal assays described above.

VI. Conclusion

Throughout this disclosure, various publications, patents and published patent specifications may be referenced by an identifying citation. The disclosures of these publications, patents and published patent specifications are hereby incorporated by reference into the present disclosure to more fully describe the state of the art to which this invention pertains.

While the invention has been described in terms of particular embodiments and illustrative figures, those of ordinary skill in the art will recognize that the invention is not limited to the embodiments or figures described. In particular, various methods and systems have been described herein with respect to particular examples involving specific assays and compounds. It will be understood that other applications, assays, and compounds may be used in accordance with other embodiments of the present invention.

In some of the embodiments described above, a single reagent carrier is coupled with a single assay carrier to load the liquid from the reagent carrier to the assay carrier. However, in other embodiments, the present invention may be implemented in other ways. For example, two reagent carriers may be coupled with the same assay simultaneously, each reagent carrier loading or retrieving compounds at the same time as the other. In addition, in various embodiments, the metering carriers and the reagent carriers may be loaded with either individual reagents (IR) or universal reagents (UR).

It will be understood that when a feature is described as being made hydrophobic or hydrophilic, this can be accomplished by forming the feature from a hydrophobic or hydrophilic material or by depositing a hydrophobic or hydrophilic layer or substance onto a surface of the feature.

The above description of the preferred embodiments of the invention has been presented for the purposes of illustration and description. It is not intended to be exhaustive or to limit the invention to the precise form disclosed. Therefore, it should be understood that the invention can be practiced with modification and alteration within the spirit and scope of the appended claims. The scope of the invention should not be determined with reference to the above description, but instead should be determined with reference to the appended claims and the full scope of their equivalents.

What is claimed is:

1. A method of performing biochemical assays, comprising:
loading a first plurality of reservoirs on a first liquid carrier with a first plurality of compounds;
coupling a second carrier with the first carrier, the second carrier having a second plurality of reservoirs configured to couple with the first plurality of reservoirs and containing a second plurality of compounds;
transferring at least a portion of the first plurality of compounds to the second plurality of reservoirs; and
separating at least one component from the second plurality of reservoirs.

2. The method of claim 1, wherein said separating at least one component from the second plurality of reservoirs comprises:
coupling a capture carrier having a plurality of protrusions with the second carrier such that each protrusion is inserted into one of the second plurality of reservoirs, each protrusion on the capture carrier having a capture component for capturing a target molecule; and
decoupling the capture carrier from the second carrier, wherein at least one of the capture components has captured a target molecule from at least one reservoir in the second plurality of reservoirs.

3. The method of claim 2, further comprising:
coupling the capture carrier with a third carrier having a third plurality of reservoirs configured to receive the protrusions on the capture carrier.

4. The method of claim 3, further comprising:
reacting at least one target molecule captured by the capture carrier with a reagent contained in the the third plurality of reservoirs.

5. The method of claim 3, further comprising:
releasing at least one target molecule into at least one reservoir from the third plurality of reservoirs.

6. The method of claim 2, further comprising detecting the presence of the captured target molecule on the capture carrier.

7. The method of claim 1, wherein:
each reservoir in the second plurality of reservoirs comprises a through-hole containing a capture probe configured to capture a target molecule; and
said separating at least one component from the second plurality of reservoirs comprises:
capturing at least one target molecule with at least one of the capture probes; and
flushing at least a portion of the contents of the second plurality of reservoirs while retaining at least one capture probe with the target molecule in the second carrier.

8. The method of claim 1, further comprising:
reacting the separated component with a reagent; and
detecting the reaction of the separated component with the reagent.

9. A method of performing biochemical analysis, comprising:
coupling a first carrier with a second carrier, the first carrier having a plurality of protrusions, each protrusion being provided with a capture component, and the second carrier having a plurality of reservoirs containing a plurality of compounds and being configured such that each of the plurality of reservoirs receives at least one of the protrusions;
uncoupling the first carrier from the second carrier; and
retrieving at least one target component from the plurality of reservoirs with at least one capture component.

10. The method of claim 9, wherein:
for each of the plurality of protrusions, said capture component is a cavity formed on a portion of the protrusion; and
said retrieving the at least one target component comprises drawing a volume of the target component into at least one of the cavities.

11. The method of claim 9, wherein:
for each of the plurality of protrusions, said capture component is a probe attached to a portion of the protrusion; and
said retrieving the at least one target component comprises linking at least one target component to at least one of the probes.

12. The method of claim 11, further comprising:
loading an addressing component into each of the plurality of reservoirs, said addressing component comprising a capture probe and a tag;
capturing the at least one target component with at least one capture probe; and
binding at least one tag with at least one probe.

13. The method of claim 11, further comprising:
loading a detection component into each of the plurality of reservoirs, said detection component comprising a detection probe and a label;
binding at least one detection probe with the at least one target component; and
after said retrieving the at least one target component, detecting the presence of at least one label.

14. The method of claim 9, wherein:
said coupling the first carrier with the second carrier comprises coupling at least two of the protrusions with each of the plurality of the reservoirs.

15. The method of claim 14, wherein:
said retrieving at least one target component from the second carrier comprises retrieving a first target component with a first capture component and retrieving a second target component with a second capture component in the same reservoir.

16. A system for biochemical analysis, comprising:
a first carrier, comprising:
a first substrate;
a plurality of reservoirs in the substrate for retaining a first plurality of compounds; and
a second carrier, comprising:
a second substrate;
a plurality of projections on the second substrate, each projection having a distal end provided with a receiving feature for receiving a component from the plurality of reservoirs when the first carrier and the second carrier are coupled.

17. The system of claim 16, wherein the receiving feature is a cavity configured to retain a liquid compound.

18. The system of claim 16, wherein the receiving feature is a capture reagent immobilized on the distal end of the projection, said capture reagent being adapted to selectively bind to a target compound.

19. The system of claim 16, further comprising:
a third carrier, comprising:
a third substrate;
a plurality of projections on the third substrate, each projection having a distal end provided with a projection reservoir for delivering a reagent to the plurality of reservoirs when the first carrier and the third carrier are coupled.

20. The system of claim 19, wherein the projection reservoir is a cavity configured to retain a liquid reagent.
